(19) United States
(12) Patent Application Publication
(10) Pub. No.: US 2026/0284053 A1
(43) Pub. Date: Sep. 24, 2026

(54) Liquid Formulation of Belumosudil

(71) Applicant: Kadmon Corporation, LLC, Morristown, NJ (US)

(72) Inventors: Olivier Schueller, Cambridge, MA (US); Galit Regev, Cambridge, MA (US); Mark Beville, Ruddington (GB); Nazim Kanji, Ruddington (GB)

(73) Assignee: Kadmon Corporation, LLC, Morristown, NJ (US)

(21) Appl. No.: 19/685,992

(22) Filed: May 22, 2026

Related U.S. Application Data
(63) Continuation of application No. 19/113,101, filed on Sep. 20, 2023.

(30) Foreign Application Priority Data
Sep. 21, 2022 (WO) PCT/US2022/44256

(51) Int. Cl. A61K 31/517; A61K 9/00; A61K 9/10; A61K 47/02; A61K 47/12; A61K 47/26; A61K 47/32; A61K 47/34; A61P 37/06

(52) U.S. Cl. CPC A61K 31/517; A61K 9/0053; A61K 9/10; A61K 47/02; A61K 47/12; A61K 47/26; A61K 47/32; A61K 47/34; A61P 37/06

(57) ABSTRACT

The present disclosure provides liquid formulations comprising belumosudil, processes for preparing a liquid formulation of belumosudil, and liquid pharmaceutical compositions comprising belumosudil which may be used in the treatment of subjects.

### CROSS-REFERENCE TO RELATED APPLICATIONS

This application claims priority to PCT Application No. PCT/US2022/044256, filed Sep. 21, 2021, which is incorporated herein by reference in its entirety.

### TECHNICAL FIELD

The present disclosure relates to liquid formulations comprising 2-{3-[4-(1H-indazol-5-ylamino)-2-quinazolinyl]phenoxy}-N-(propan-2-yl) acetamide, also known as belumosudil. The present disclosure further relates to methods of preparing liquid formulations comprising belumosudil and methods of treating subjects with liquid formulations including adult and pediatric populations.

### BACKGROUND

Belumosudil is an oral selective rho-associated coiled-coil-containing protein kinase-2 (ROCK2) inhibitor. ROCK2 inhibition acts on the dysregulated adaptive immune system and the fibrosis that occurs because of aberrant tissue repair. Belumosudil inhibits ROCK2 and ROCK1 with IC₅₀ values of approximately 100 nM and 3 μM, respectively.

The compound belumosudil has the chemical name: 2-{3-[4-(1H-indazol-5-ylamino)-2-quinazolinyl]phenoxy}-N-(propan-2-yl) acetamide, and is represented by Formula I below:

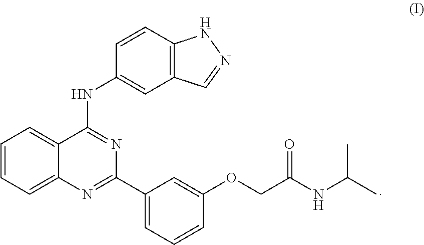 [SMILES: CC(C)NC(=O)COc1cccc(-c2nc(Nc3ccc4[nH]ncc4c3)c3ccccc3n2)c1]

Belumosudil is also known as KD025.

The mesylate salt of belumosudil is presently marketed in the United States and other countries under the tradename REZUROCK® (Kadmon Corp./Sanofi), for the treatment of patients with chronic graft-versus-host disease (cGVHD), in some instances after failure of at least two prior lines of systemic therapy. The active pharmaceutical ingredient of REZUROCK® is belumosudil mesylate salt with the molecular formula C₂₇H₂₈N₆O₅S, a molecular weight of 548.62 g/mol, and the chemical name 2-{3-[4-(1H-indazol-5-ylamino)-2-quinazolinyl]phenoxy}-N-(propan-2-yl) acetamide methanesulfonate (1:1).

The chemical structure of belumosudil mesylate is as follows:

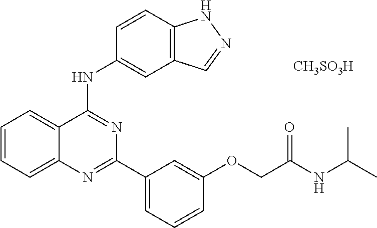

Belumosudil binds to and inhibits the serine/threonine kinase activity of ROCK1 and ROCK2. Belumosudil down-regulated proinflammatory responses via regulation of STAT3/STAT5 phosphorylation and shifting Th17/Treg balance in ex-vivo or in vitro-human T cell assays. Belumosudil also inhibited aberrant pro-fibrotic signaling, in vitro. In vivo, belumosudil demonstrated activity in animal models of cGVHD. Belumosudil is therefore useful in treating diseases, disorders and conditions regulated by ROCK including autoimmune and fibrotic disorders, acute and chronic GVHD, idiopathic pulmonary fibrosis, and moderate to severe psoriasis, among other indications.

A process for preparing belumosudil is disclosed in U.S. Pat. No. 8,357,693 (the '693 patent), specifically, in Example 82 thereof. The process disclosed in the '693 patent provides belumosudil as a crude solid product that was purified by high performance liquid chromatograph (HPLC). Belumosudil and processes for making the compound are also described in U.S. Pat. Nos. 9,815,820, 10,183,931, and 10,696,660.

Present methods of administering belumosudil involve formulating the mesylate salt of belumosudil into pharmaceutically acceptable capsules and tablets for oral administration. However, certain patients who may benefit from treatment with belumosudil are unable to, or have difficulty with, and/or disfavor swallowing solid dosage forms, for example, certain adult patients, patients with dysphagia, and/or pediatric patients. Liquid formulations of belumosudil would thus be useful for administering the compound to a broader population of patients. Liquid formulations also may provide advantages in terms of enhancing the flexibility in how the drug is administered.

Liquid formulations also may provide advantages in terms of enabling flexibility in administering a clinically recommended dose to a patient according to body weight. For example, with a liquid formulation, the amount of drug to be administered can be adjusted on a sliding scale depending upon the patient's body weight. In comparison, with the solid oral dosage form as presently marketed, each tablet comprises 200 mg of belumosudil.

Belumosudil is, however, a weakly basic compound that is practically insoluble in water. Belumosudil is also insoluble or practically insoluble in many other solvents or carriers typically used in pharmaceutical drug development. Belumosudil's low solubility presents challenges in developing liquid formulations. Moreover, liquid formulations should be homogenous upon suspension and resuspension (for example, when a single vial is used several times). To obtain a liquid formulation capable of homogenous resuspension requires the use of specific equipment, which can lead to foaming. This is especially true for hydrophobic compounds such as belumosudil. Therefore, there remains a need for liquid formulations comprising belumosudil and methods of producing homogenous preparations that are capable of resuspension that can address these challenges.

### SUMMARY

In one aspect, the present disclosure provides a liquid formulation comprising 2-{3-[4-(1H-indazol-5-ylamino)-2-quinazolinyl]phenoxy}-N-(propan-2-yl) acetamide, or a pharmaceutically-acceptable salt thereof (belumosudil). In one embodiment, the liquid formulation comprises jet-milled or pin-milled belumosudil suspended in a diluent in combination with a suspending agent and a thickener. The liquid formulation comprising belumosudil may also include a preservative, a pH adjuster as needed to achieve a pH in the range of about 2.5 to 4.0, and a sweetening agent and/or a flavoring agent.

In an additional aspect, the disclosure provides a liquid formulation comprising 2-{3-[4-(1H-indazol-5-ylamino)-2-quinazolinyl]phenoxy}-N-(propan-2-yl) acetamide, or a pharmaceutically-acceptable salt thereof (belumosudil) in combination with a suspending agent, a thickener, and an antifoaming agent. The liquid formulation comprising belumosudil may also include one or more of a preservative, a pH adjuster as needed to achieve a pH in the range of about 2.5 to 4.0, and a sweetening agent and/or a flavoring agent. In one embodiment, the liquid formulation comprises jet-milled belumosudil. In one embodiment, the antifoaming agent comprises simethicone. In one embodiment, the antifoaming agent comprises an about 30% simethicone emulsion.

Another aspect of the disclosure provides a liquid formulation of belumosudil that is produced by mixing jet-milled or pin-milled belumosudil in a vehicle wherein the vehicle comprises at least a suspending agent and a thickener in a diluent.

Another aspect of the disclosure provides a liquid formulation of belumosudil that is produced by mixing jet-milled or pin-milled belumosudil in a vehicle wherein the vehicle comprises at least an antifoaming agent in a diluent.

In another embodiment, the disclosure provides a kit comprising jet-milled or pin-milled belumosudil prepared as a dry powder and provided with a reconstitution vehicle comprising at least a suspending agent and a thickener in a diluent, wherein the belumosudil may be reconstituted before use with a reconstitution vehicle. The disclosure also provides processes for preparing a liquid formulation of belumosudil, as further described herein.

In another embodiment, the disclosure provides pharmaceutical compositions comprising an effective amount of the liquid formulation of belumosudil that are useful for treating diseases, disorders and conditions regulated by ROCK as further described herein.

### BRIEF DESCRIPTION OF THE FIGURES

FIG. 1 is a flowchart illustrating a process for preparing a liquid formulation of belumosudil.

FIGS. 2A-2B show particle size distribution (PSD) data for the pin-milled belumosudil in sterile water for irrigation, as described in Example 3.

FIG. 3A shows PSD data of a pin-milled belumosudil liquid formulation with Natrosol (sample 3A of Example 4).

FIG. 3B shows PSD data of a jet-milled belumosudil liquid formulation with Natrosol (sample 3B of Example 4).

FIG. 3C shows PSD data of a pin-milled belumosudil liquid formulation with hypromellose K4M (sample 4A of Example 4).

FIG. 3D shows PSD data of a pin-milled belumosudil liquid formulation with povidone (sample 6A of Example 4).

FIG. 4 shows sedimentation assay results for samples obtained from six liquid formulations comprising belumosudil including three formulations comprising pin-milled belumosudil (samples 13A-15A) and three formulations comprising jet-milled belumosudil (samples 13B-15B), as described in Example 6.

FIG. 5 is a flowchart illustrating an additional process for preparing a liquid formulation of belumosudil.

FIG. 6 is a flowchart illustrating yet a further process for preparing a liquid formulation of belumosudil.

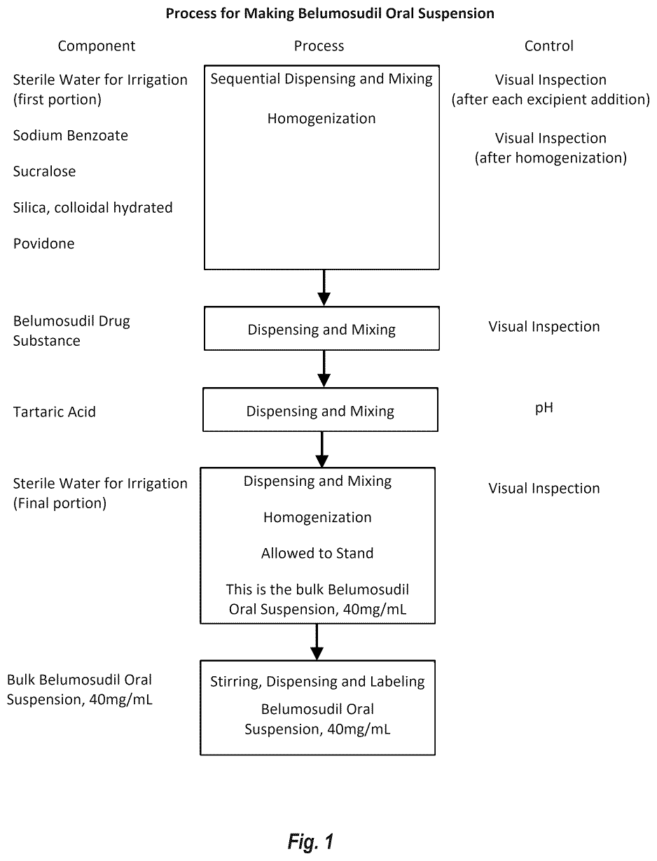

### DETAILED DESCRIPTION

### Definitions

“About” as used herein includes the exact amount modified by the term, about, as well as an amount that would be expected to be within experimental error, such as for example, within 15%, 10%, or 5%, depending on the context. For example, “about 200 mg” means “200 mg” and also a range of mgs that is within experimental error, e.g., plus or minus 15%, 10%, or 5% of 200 mg. As used herein, the term “about” may be used to modify a range and also, a particular value.

“Administering” or “administered to” as used herein (for example, with reference to administration of belumosudil or liquid formulations containing belumosudil) refers to the act of prescribing medicine(s) containing belumosudil for the subject to take during treatment, the act of prescribing a protocol of medicines to be taken by a subject, the act of dispensing the medicine(s) to the subject, the act of reconstituting powdered belumosudil in a liquid formulation, and/or the act of physically receiving or ingesting the medicine(s). Thus, the belumosudil can be “administered” by a physician or other medical professional who writes prescriptions for belumosudil; and/or by a pharmacist who fills said prescriptions, and/or by a person who prepares a reconstituted liquid formulation using powdered belumosudil; and/or by the patient or subject who ingests the belumosudil and/or his or her partner or caretaker who delivers the belumosudil to the subject.

“API” means “active pharmaceutical ingredient” which is synonymous herein with the definition of belumosudil (or KD025).

“Allogeneic hematopoietic stem cell transplantation (allo-HSCT)” also called bone marrow transplantation or stem cell transplantation, or “allogeneic hematopoietic cell transplantation (allo-HCT)” refers to a procedure where hematopoietic cells from a donor are grafted into a recipient who is not an identical twin. The source of hematopoietic stem cells for allogeneic transplantation may be peripheral blood stem cells (PBSC) or bone marrow (BM). In some circumstances umbilical cord blood may be used. The donor and recipient may be matched at the human leukocyte antigen (HLA) genes, such as siblings. The donor and recipient may be a parent and a child who are only half-matched (haploidentical).

“Belumosudil” as used herein, unless the context clearly indicates otherwise, may cover the compound belumosudil in any form as well as pharmaceutically acceptable salts thereof. The term “belumosudil” refers both to the compound belumosudil (for example, in the free base form, amorphous form, or crystalline form), to pharmaceutically acceptable salts of belumosudil, for example, the mesylate salt form as used in as REZUROCK®, and to any form of belumosudil that may be used in preparing a liquid formulation or pharmaceutical composition for administering the compound to a patient.

“Clinical endpoint” or “study endpoint” refers to an event or outcome in a clinical trial that can be measured objectively to determine outcomes and potential beneficial effects of the drug or administration protocol as designed in the clinical trial. Examples of clinical endpoints include the following. Overall response rate (ORR) is the percentage of people in a study or treatment group who have a partial response (PR) or complete response (CR) to the treatment within a certain period of time. Failure-free survival (FFS) means the time from the first dose of belumosudil to a failure event, or the interval between the start of belumosudil and the addition of a new cGVHD therapy, relapse of the underlying disease, or nonrelapse mortality (NRM). Overall survival (OS) means the length of time from either the date of diagnosis or the start of treatment for a disease. Duration of response (DOR) means from the time of initial response (e.g., PR or CR) until documented progression from best response of cGVHD, time from initial response to start of additional systemic cGVHD therapy, or death. Time to next treatment (TTNT) means time to initiation of a subsequent systemic cGVHD therapy.

“Clinically recommended amount” or “clinically recommended dosage” refers to the amount or dosage of belumosudil that has been recommended and/or approved for administration to a subject by those skilled in the field of medicinal chemistry to treat the disease state in question following clinical trials, for example, as set forth in publications, clinical trial results, and on the approved drug label. In one embodiment, a clinically recommended dosage for belumosudil, without administration of CYP3A inducers or PPIs, as indicated on the drug label for belumosudil, is 200 mg once daily.

“Co-administration,” “in combination with,” and/or “co-administered,” as used herein with reference to administration of belumosudil with other substances (for example, CYP3A Inducers and/or PPIs) means that during the course of the patient's treatment with belumosudil, the patient is also receiving one or more dosages of one or more other treatments. The compounds need not be administered concomitantly with, or on the same day, as the belumosudil to be considered as being “co-administered” under this definition.

“High-fat, high-calorie meal” refers to a meal containing about 800 to 1,000 calories with approximately 50% of total caloric content of the meal from fat food. For example, in one embodiment, a high-fat breakfast may consist of hash browns, bacon, fried egg, white bread, and 240 mL of full fat milk.

“Homogenizer” refers to a piece of laboratory or industrial equipment used for the homogenization of various types of material, such excipients, diluent, and API. One skilled in the field will appreciate that there are many different types of homogenizing equipment, including Cell Lysor, Disperser, High Shear Mixer, Homogenizer, Polytron, Rotor Stator Homogenizer, Sonicator, Tissue Tearor, and the like. Homogenizers can perform mixing using different types of paddles, rotor-stator assemblies, and screens, and at varying shear rates and rotation speeds. One advantageous homogenizer is the SILVERSON® homogenizer. One advantageous homogenizer assembly includes a high shear screen.

“Immunosuppressive therapy” (IST) refers to therapy that is typically administered for at least six months after allo-HSCT to try to prevent GVHD. Examples of IST's include sirolimus, prednisone and calcineurin inhibitors such as tacrolimus and cyclosporine.

“Label claim” refers to the amount or dosage of API corresponding to that which is indicated in an approved drug label. For example, in the United States, the currently approved drug label for REZUROCK® states that the recommended dosage is 200 mg taken orally once daily with food and 200 mg twice daily when co-administered with strong CYP3A inducers and PPIs. The equivalent dose of belumosudil in a liquid formulation (corresponding to the 200 mg label dose), is calculated as 40 mg/mL freebase, which is equivalent to 48.496 mg/mL belumosudil mesylate when adjusted with a 1.2124 salt correction factor.

Lee Symptom Scale (LSS) summary score measures the effect on patients' functioning and well-being. The Lee Symptom Scale is a 30-item scale developed to measure the symptoms of cGVHD and is described in Lee S J, et al., Development and validation of a scale to measure symptoms of chronic graft-versus host disease. Biol Blood Marrow Transplant 2002; 8:444-452.

“Line of treatment” or “line of therapy” describes the sequence or order in which different therapies are given to a patient as the patient's disease progresses. Initial treatment (first-line therapy) may not work or may stop working after a period. After first-line therapy is discontinued, a second different treatment (second-line therapy) may be given. Subsequent lines of therapy may be given when a second-line therapy does not work or stops working. Some patients may be administered multiple lines of therapy over the course of a disease.

First-line therapy for National Institutes of Health (NIH)-defined moderate to severe chronic graft-versus-host disease (cGVHD) may be corticosteroids alone or in combination with sirolimus or a calcineurin inhibitor. (Carpenter P A, et al.: A phase II/III randomized, multicenter trial of prednisone/sirolimus versus prednisone/sirolimus/calcineurin inhibitor for the treatment of chronic graft-versus-host disease: BMT CTN 0801. Haematologica 103:1915-1924, 2018).

Examples of corticosteroid therapies for treatment of cGVHD include, but are not limited to, prednisone, prednisolone, methylprednisolone, and budesonide. Examples of prior systemic therapies for treating cGVHD include, but are not limited to, prednisone, tacrolimus, extracorporeal photopheresis (ECP), sirolimus, ibruitinib, ruxolitinib, mycophenolate mofetil (MMF), rituximab, methotrexate (MTX), cyclosporine, imatinib, ixazomib, and ofatumumab.

“Liquid formulation” or “liquid” as used herein means that the formulation is substantially in a liquid state rather than a solid (or gaseous) state and includes suspensions where solid particles (e.g., particles of belumosudil), are spread throughout the liquid without dissolving in it.

“Myeloablative transplant” refers to a transplantation process using very high doses of chemotherapy or radiation prior to transplantation with autologous or allogeneic hematopoietic stem cells. A non-myeloablative transplant, or reduced intensity transplant, involves the patient having less intensive chemotherapy before transplantation with allogeneic hematopoietic stem cells.

“NIH lung symptom score” or “NIH cGVHD lung score” is a clinical symptom-based score ranging from 0 to 3. A Score 0 is used for no symptoms, Score 1 is used for symptoms of shortness of breath with stairs, Score 2 is used for symptoms of shortness of breath on flat ground, and Score 3 is used for shortness of breath at rest or requiring oxygen.

“Or” is used in the inclusive sense (equivalent to “and/or”) unless the context requires otherwise.

“Patient” or “subject” as used herein includes an animal or a human; in one embodiment, the term “patient” refers to a human subject.

“Pediatric patient” as used herein means a non-adult patient (i.e., less than 18 years of age); in one embodiment, it means a patient less than age 12; in another embodiment, it refers to a patient between the ages of 3 months and 12 years of age.

“Pharmaceutically-acceptable salts” refers to non-toxic, inorganic and organic acid addition salts of belumosudil. In one embodiment, the pharmaceutically-acceptable salt of belumosudil herein is the mesylate salt.

“Povidone” (also referred to as “povidon”) refers to a water-soluble polymer made from the monomer N-vinylpyrrolidone which is also commonly known as polyvinylpyrrolidone or polyvidone. Povidone has the molecular formula of (C₆H₉NO)ₙ and appears as a white to slightly off-white powder. The k number associated with the povidone name refers to the mean molecular weight of the povidone. Thus, povidone 90 has a higher molecular weight than povidone K30.

“Protocol” as used herein refers to the methods or plan that is used to administer the belumosudil to a subject in need of treatment. The term “protocol” is intended to encompass the overall, detailed plan of care for a patient, as well as individual or partial steps that are part of the overall plan. For example, a protocol may include the dosages of belumosudil that the patient will be (or is) receiving, the combination of drugs the patient receives, the timing and method of administration of the belumosudil (for example, considering DDIs, food effects, and impact different formulations or modes of delivery may have on absorption and bioavailability), and management of side effects, as well as the overall plan encompassing the dosages, combinations, timing and methods of administration, and side effects, considered together.

“Proton pump inhibitor” or “PPI” refers to a drug that inhibits the stomach's H⁺/K⁺ ATPase proton pump and causes a reduction in stomach acid production. As PPIs reduce stomach acid production, they can increase the pH of the stomach which impact the solubility and potentially the bioavailability of orally delivered medicines. Examples of PPIs include omeprazole, lansoprazole, dexlansoprazole, esomeprazole, pantoprazole, rabeprazole, and ilaprazole.

“Silica” or “silica agent” when used herein refers to any silica product that may be used as a glidant and/or anti-caking agent such as silicon dioxide, fumed silica, colloidal hydrated silica, precipitated silica, and magnesium aluminum silicate and includes silica products sold under the tradenames SYLOID® and AEROSIL®.

AEROSIL® 200 is a hydrophilic fumed silica product containing more than 99.8% SiO₂ and having a specific surface area of 200 m2/g.

SYLOID® 244 FP is an alternative silica (SiO₂) product sold in the form of an amorphous white free flowing powder available from W.R. Grace & Co. (Conn. USA). SYLOID® 244 FP silica is a high pore volume silica gel with a large internal surface area. It has a strong affinity for moisture and organic based liquids. SYLOID® 244 FP silica can adsorb up to 1.6 ml of liquid per gram. SYLOID® 244 FP silica is advantageous as a glidant, tableting-aid and carrier for pharmaceutical and personal care products.

“Simethicone” (also referred to as “simethicone emulsion”) refers to an antifoaming agent. Simethicone has the molecular formula of C₆H₁₈O₄Si₃ and is available as a 30% aqueous emulsion that appears as an off-white homogenous liquid. An advantageous 30% simethicone emulsion is a Medical Antifoam C Emulsion made with 30% simethicone USP, methylcellulose, sorbic acid and water.

“Steroid-refractory” (SR) cGVHD is defined as cGVHD progression while on steroids or corticosteroids; in one embodiment, while on prednisone.

“Suspension” refers to a mixture of finely distributed solid in a liquid wherein the solid is not dissolved in the liquid. The term “liquid formulation” or “formulation” as used herein includes a suspension. For example, the terms “liquid formulation comprising belumosudil,” “liquid formulation of belumosudil” and/or “belumosudil liquid formulation” are intended to encompass a suspension of belumosudil or a heterogenous or homogenous mixture wherein solid belumosudil particles are distributed in the liquid but not dissolved therein.

A “therapeutically effective amount” of an API means an amount which, when administered to a human for treating a disease (for example, cGVHD), is sufficient to effect treatment for the disease state being treated. As applied to cGVHD in a human, “treating” or “treatment” includes (1) reducing the risk of developing cGVHD and/or inhibiting cGVHD, i.e., arresting or reducing the development of cGVHD or its clinical symptoms; and (2) relieving cGVHD, i.e., causing regression, reversal, or amelioration of the cGVHD or reducing the number, frequency, duration or severity of its clinical symptoms.

The therapeutically effective amount of an API may vary depending upon the health and physical condition of the subject to be treated, the extent of disease progression, the assessment of the medical situation, and other relevant factors. It is expected that the therapeutically effective amount may fall within a range that can be determined through trial and through reference to clinical trial data and results, for example, as described in the Examples herein and in scientific literature.

The phrase “pharmaceutically-acceptable carrier” as used herein means a pharmaceutically-acceptable material, composition or vehicle, such as a solid filler, diluent, excipient, or manufacturing aid. Each carrier must be “acceptable” in the sense of being compatible with the other ingredients of the formulation and not injurious to the patient.

### Overview

The instant disclosure provides a useful liquid formulation comprising belumosudil meeting an acceptable Quality Target Product Profile (QTPP) for use in administering belumosudil to patients in a manner that does not require swallowing of a solid dosage form.

To provide the liquid formulation comprising belumosudil as disclosed herein, various excipients were investigated including viscosity modifiers, preservatives, suspending agents, pH modifiers, sweeteners, thickeners, and antifoaming agents. Physical stability issues encountered with the agents were investigated over time and at various temperatures and pH systems. Applicant discovered that satisfactory dispersion and resuspendability of belumosudil could be achieved at suitable levels of inclusion to meet concentrations corresponding to adult label claims and for the pediatric population with a colloidal silica system.

Formulations were assessed with combinations of suspending/thickening agents, preservatives, sweeteners, flavors, and antifoaming agents to identify the most advantageous systems for a liquid formulation comprising belumosudil.

An assessment of jet-milled and pin-milled belumosudil was also carried out to select the most appropriate input material for use in the liquid formulation comprising belumosudil. A comparison of formulations manufactured with the alternative milling technologies for belumosudil initially suggested that pin-milled API would offer the most appropriate material for use in the formulation and that jet-milled API was problematic and presented various barriers. However, upon further investigation, applicant discovered that jet-milled API offered the preferred form for achieving uniform homogeneity and meeting the QTPP.

The knowledge of the belumosudil behavior in a liquid combined with the understanding of the importance of the ingredients played an important role in the development of this stable formulation. Jet-milled API may be used in this formulation instead of pin-milled API to lower the amounts of sedimentation and foaming.

Additionally, a suspending agent may be used, such as silica or colloidal silica to minimize the sedimentation and in combination with povidone (or polyvinylpyrrolidone [PVP]) to improve dispersion. Povidone is a polymer and thickening agent that can assist with maintaining the viscosity of the medium.

Additionally, a preservative may be used. In fact, as disclosed herein, using a preservative method was found to provide advantages in protecting a liquid formulation comprising belumosudil. Sodium benzoate was selected as an effective preservative for use in the formulation after experimentation. Sodium benzoate has both bacteriostatic and antifungal properties attributed to undissociated benzoic acid. Accordingly, a liquid formulation of belumosudil without a preservative method would be less advantageous for preparing a ready-to-use liquid pharmaceutical formulation.

Liquid formulations that are suspensions should be robust, such that they are homogenous upon suspension and resuspension, and ideally do not foam upon mixing, including during manufacturing. Such formulations can be difficult to prepare. During formulation manufacture, mixing at high speeds and with specialized mixing equipment (such as paddle shape and screens), can be used to reduce particle agglomeration and provide a homogeneous and stable dispersion. A side effect of mixing at high speeds, especially with a hydrophobic drug product, is increased foaming. Compositions and manufacturing process designed to overcome the foaming issue can result in a suspension with the desired features.

Additionally, an antifoaming agent may be used. In fact, as disclosed herein, using an antifoaming agent was found to provide advantages in liquid formulations comprising belumosudil that are homogeneous and stable. An about 30% simethicone emulsion was selected as an effective antifoaming agent for use in formulations after experimentation. Accordingly, a liquid formulation of belumosudil without an antifoaming method would be less advantageous for preparing a ready-to-use liquid pharmaceutical formulation that that can be re-suspended multiple times in a homogeneous manner.

An assessment of mixing processes was also carried out to select the most appropriate manufacturing conditions for use in preparing the liquid formulation comprising belumosudil. A comparison of formulations manufactured using alternative processing steps initially suggested that using a two-vessel (side phase) process, incorporating a hold time step, and using relatively low-speed mixing (that is, using rotation speeds less than about 1000 rpm) would offer the most appropriate manufacturing process for preparing the liquid belumosudil formulation. However, upon further investigation, applicant discovered that addition of an antifoaming agent to the formulation and using higher speed mixing via a homogenizer with a high shear screen to manufacture the formulation offered the preferred form for achieving uniform homogeneity and meeting the QTPP.

In one embodiment, a homogenization step may be used to improve the uniformity of the formulation. Homogenization may be performed using a standard or a high-shear rotor-stator assembly and may include use of a shear screen. In an embodiment, the homogenization is performed at rotation speeds between about 900 and 10000 rpm, such as at least about 1000 rpm, between about 1000 and 8000 rpm, between about 4000 and 6000 rpm, or about 5000 rpm. In an embodiment, the homogenization is performed using a high shear screen. In an embodiment, the homogenization is performed using a paddle. In an embodiment, the homogenization is performed using a rotor and stator.

Flavor and sweetener combinations that showed improved palatability when compared to the unflavored and unsweetened control system were identified from a human volunteer taste study and development activities were performed to optimize the thickener/suspending system for belumosudil.

Agents and excipients for use in the liquid formulation comprising belumosudil are commercially available. For example, silica suspending agents sold under the tradename SYLOID® are available from W.R. Grace & Co. (Conn. USA); silica products sold under the tradename AEROSIL® are commercially available from Evonik Degussa (Essen, Germany) and/or Azelis Ltd (Hertford, UK); thickening agents povidone K30 and povidone K90F are commercially available from BASF Corp. (Florham Park, NJ, USA); sucralose and sodium benzoate are commercially available from Merck KGaA (Darmstadt, Germany); flavoring agents such as lemon and tropical fruit blend flavor are commercially available from Givaudan International SA (Switzerland) and/or IMCD Group; and antifoaming agents such as simethicone are commercially available from DuPont (Wilmington, DE, USA) and Dow Corning (Midland, MI, USA).

Alternative formulations were identified, and a lead formulation was selected following stability testing. The stability of the formulation is an important attribute for its viability in practical applications particularly where the goal is to deliver the formulation comprising belumosudil in a pre-prepared, liquid form. To that end, applicants evaluated various alternative belumosudil formulations to assess their chemical stability (absence of degradation); solution stability (limited change in pH over time); physical stability (absence of irreversible sedimentation or ability to reconstitute the formulation via agitation); and microbiological stability (use of preservative to ensure no microbial/bacterial growth).

The belumosudil formulation disclosed herein enables flexible dosing by enabling easy variation in dose volume according to the age and/or body weight of the patient. In one embodiment, belumosudil is administered with a weight-based dosing regime wherein subjects receive approximately the same dose on a mg/kg basis. Such a weight-based dosing regime is not available with a fixed dose tablet.

This liquid formulation comprising belumosudil according to the disclosure herein is suitable for patients of all ages including adults and children, including adults and children who are unable to, have difficulty with, or disfavor swallowing solid oral dosage forms. For example, liquid formulations comprising belumosudil suitable for children from age 3 months to 12 years, or 3 months to 18 years, are contemplated. The liquid formulation may be manufactured on a large scale for use in marketing and relative bioavailability assessments and compared with solid oral dosage forms, such as the 200 mg tablet approved for marketing.

In one embodiment, the disclosure provides a liquid, suspension form of belumosudil that may be administered by the oral route, for example, to adult patients who are unable to, having difficulty with, and/or prefer not to swallow oral tablets or capsules and/or to pediatric patients.

In one embodiment, the liquid formulations comprising belumosudil disclosed herein may be provided in a ready-to-use liquid form wherein the belumosudil is already distributed through the liquid vehicle. In other embodiments, the belumosudil may be prepared and manufactured as a dry powder and provided as a kit with the reconstitution vehicle, wherein the belumosudil is reconstituted before use with the vehicle.

### Exemplary Embodiments

In one embodiment, a liquid formulation is provided having the components set forth in Table 1.

TABLE 1
Formulation Components

| Component | % w/w |
|---|---|
| belumosudil (API) | 2-8 |
| Preservative | 0.02-0.08 |
| Sweetening Agent | 0.1-0.4 |
| Suspending Agent | 0.206-0.824 |
| Thickener | 2.061-8.247 |
| pH Adjuster | q.s to pH 2.5 to 4.0 |
| Diluent | q.s to volume |

In one embodiment, belumosudil mesylate is used in the formulation described in Table 1. In one embodiment, the belumosudil mesylate has been jet-milled and/or pin-milled. In some embodiments, the belumosudil (or belumosudil mesylate), has been jet-milled.

In another embodiment, the preservative used in the Table 1 formulation is sodium benzoate.

In another embodiment, the sweetening agent used in the Table 1 formulation is sucralose.

In another embodiment, the suspending agent used in the Table 1 formulation is a silica product selected from colloidal silica, silicon dioxide, and fumed silica. In another embodiment, the suspending agent is colloidal silica; in one embodiment, the suspending agent is SYLOID®244FP. In another embodiment, the suspending agent is AEROSIL® 200.

In another embodiment, the thickener used in the Table 1 formulation is a water-soluble polymer made from the monomer N-vinylpyrrolidone. In another embodiment, the thickener is povidone. In another embodiment, the thickener is selected from povidone K30 and povidone 90F. In another embodiment, the thickener is povidone 90F.

In another embodiment, the pH adjuster used in the Table 1 formulation is tartaric acid. In another embodiment, the pH adjuster is added to achieve a pH in the range of 3.0±0.5.

In another embodiment, the diluent used in the Table 1 formulation is sterile water for irrigation.

In one embodiment, a flavor is added to the formulation described in Table 1. In one embodiment, the flavor is added to the liquid formulation when it is prepared, together with other excipients and/or ingredients. In another embodiment, the flavor is stored and/or provided in a separate container and optionally added to the liquid formulation at about the point of administration, as needed and/or desired.

In one embodiment, the flavor is selected from those commercially available and suitable for use in pharmaceuticals.

In another embodiment, the flavor is selected from tropical fruit blend flavor, lemon flavor, and orange flavor; in another embodiment, the flavor is tropical fruit blend flavor.

In one embodiment, the flavor is Lemon Flavor PHS-135460 available from IMCD Group; in another embodiment, the flavor is Tropical Fruit Blend Flavor PHS-145298 available from IMCD Group.

In another embodiment, a liquid formulation is provided having the components set forth in Table 2 below.

TABLE 2
Belumosudil Formulation Components

| | | | | Quantity per |
|---|---|---|---|---|
| Component | Function | % w/w | mg/5 mL dose | 2600 g batch (g) |
| Belumosudil | Drug substance | 4.75 | 242.48^{a} | 123.63 |
| Mesylate | | | (200 mg | |
| | | | freebase) | |
| Sodium Benzoate | Preservative | 0.05 | 2.50 | 1.27 |
| Sucralose | Sweetening | 0.15 | 7.50 | 3.82 |
| | Agent | | | |
| SYLOID ®244FP | Suspending | 0.49 | 25.00 | 12.74 |
| | Agent | | | |
| Povidone 90F | Thickener | 4.90 | 250.00 | 127.45 |
| Tartaric Acid | pH Adjustment | q.s. to pH | q.s. to pH | q.s. to pH |
| | | 3 ± 0.5 | 3 ± 0.5 | 3 ± 0.5 |
| Sterile Water | Vehicle | q.s. to | q.s. to | q.s. to |
| for Irrigation | | 100.00 | 5.10 g | 2600.00 |
| Total | — | 100.00 | 5100.00 | 2600.00 |

^{a}The drug substance amounts (mg) are given as belumosudil mesylate salt, with the free base equivalent in parentheses. The label dosage is specified on the basis of free base. If required, potency adjustment may be performed.

In one embodiment, an antifoaming is added to the formulation described in Table 1 or Table 2. In one embodiment, the antifoaming agent comprises simethicone. In another embodiment, the antifoaming agent is an about 30% simethicone emulsion.

In another embodiment, a liquid formulation is provided having the components 2-{3-[4-(1H-indazol-5-ylamino)-2-quinazolinyl]phenoxy}-N-(propan-2-yl) acetamide or a pharmaceutically acceptable salt thereof (belumosudil), a suspending agent, a thickener, a preservative, a pH adjuster as needed to achieve a pH in the range of about 2.5 to 4.0, a sweetening agent, an anti-microbial agent, and an antifoaming agent.

In another embodiment, a liquid formulation is provided having the components set forth in Table 3 below.

TABLE 3
Belumosudil Formulation Components

| | Concentration |
|---|---|
| Component | (% w/w) |
| belumosudil (API) | 2-8 |
| Preservative/Anti-microbial agent | 0.02-0.08 |
| Sweetening agent | 0.1-0.4 |
| Suspending agent | 0.2-0.8 |
| Thickening agent | 2.1-8.2 |
| Antifoaming agent | 0.25-4.0 |
| pH adjusting agent | q.s. to pH 2.5 to 4.0 |
| Vehicle | q.s. to 100 |

In one embodiment, belumosudil mesylate is used in the formulation described in Table 3. In one embodiment, the belumosudil mesylate has been jet-milled and/or pin-milled. In some embodiments, the belumosudil (or belumosudil mesylate) has been jet-milled.

In another embodiment, the preservative used in the Table 3 formulation is sodium benzoate.

In another embodiment, the sweetening agent used in the Table 3 formulation is sucralose.

In another embodiment, the suspending agent used in the Table 3 formulation is a silica product selected from colloidal silica, silicon dioxide, and fumed silica. In another embodiment, the suspending agent is colloidal silica; in one embodiment, the suspending agent is SYLOID®244FP. In another embodiment, the suspending agent is AEROSIL® 200.

In another embodiment, the thickener used in the Table 3 formulation is a water-soluble polymer made from the monomer N-vinylpyrrolidone. In another embodiment, the thickener is povidone. In another embodiment, the thickener is selected from povidone K30 and povidone 90F. In another embodiment, the thickener is povidone 90F.

In another embodiment, the antifoaming agent used in the Table 3 formulation is simethicone. In another embodiment, the antifoaming agent is an about 30% simethicone emulsion. In one embodiment, the antifoaming agent is a 30% simethicone emulsion Medical Antifoam C Emulsion.

In another embodiment, the pH adjuster used in the Table 3 formulation is tartaric acid. In another embodiment, the pH adjuster is added to achieve a pH in the range of 3.0±0.5. In some embodiments, the pH is added to achieve a pH in the range of 2.5-4.0. In some embodiments, the pH adjuster is added to achieve a pH in the range of 2.7-3.3.

In another embodiment, the diluent used in the Table 3 formulation is sterile water for irrigation. In another embodiment, the diluent used in the Table 3 formulation is purified water.

In another embodiment, a liquid formulation is provided having the components set forth in Table 4 below.

TABLE 4
Belumosudil Formulation Components

| Component | Function | % w/w |
|---|---|---|
| Belumosudil Mesylate | Active Pharmaceutical | 4.755 |
| (Jet milled) | Ingredient | |
| Sodium Benzoate | Preservative/Anti- | 0.05 |
| | microbial agent | |
| Sucralose | Sweetening Agent | 0.15 |
| SYLOID ®244 FP | Suspending Agent | 0.49 |
| Povidone PVP K-90 | Thickening Agent | 4.90 |
| 30% Simethicone | Antifoaming agent | 2.00 |
| emulsion (Medical | | |
| Antifoam C Emulsion) | | |
| Tartaric Acid | pH adjusting agent | q.s. to pH 3.0 |
| Purified Water | Vehicle | q.s. to 100.00 |

In another embodiment, a liquid formulation is provided having the components in Table 1, Table 2, Table 3, or Table 4 and which meets a Quality Target Product Profile (QTPP), having the following requirements: 200 mg belumosudil dose (free base equivalent), single oral 5 mL dose (dose is expressed as the free base equivalent and is based on the salt correction factor of 1.2124, wherein 200 mg free base is equivalent to 242.48 mg mesylate salt); no taste/mouth feel issues, a uniform color; and sufficient shelf-life for packaging and distribution optionally with refrigeration (in some embodiments, at 2-8° C.).

In one embodiment, the disclosure provides a liquid formulation comprising belumosudil with a concentration of belumosudil equivalent to about 40 mg/mL freebase. In another embodiment, a more dilute formulation is provided to facilitate administration of lower doses (for example, a low dose administered via a high concentration formulation would require a low volume that may be more difficult to measure). In another embodiment, a more concentrated formulation is provided to facilitate administration of higher doses (for example, a high dose administered via a low concentration formulation would require a higher volume that may be more difficult for the patient to receive.)

Accordingly, in one embodiment, the formulation comprises about 242 mg of belumosudil mesylate (equivalent to 200 mg free base) per each 5 mL of the liquid formulation. In another embodiment, the formulation is diluted to provide about 242 mg of belumosudil mesylate (equivalent to 200 mg free base) per each 10 mL of liquid formulation. In one embodiment, a 5 mL dose comprises an equivalent dose of about 100 mg freebase belumosudil. In another embodiment, the formulation is diluted further to provide about 242 mg of belumosudil mesylate (equivalent to 200 mg free base) per each 20 mL of the liquid formulation. In one embodiment, a 5 mL dose comprises an equivalent dose of about 50 mg freebase belumosudil.

In another embodiment, the formulation as described in Table 1, Table 2, Table 3, or Table 4 is processed with a homogenization step. In one embodiment, the formulation is prepared with use of a mixing vessel, and the vessel is placed in a homogenizer which is operated to homogenize the contents during at least one step of the formulation preparation. In one embodiment, the homogenization is performed at a rotation speed between about 4000 and 6000 rpm. In an embodiment, the homogenization is performed using a high shear screen. In an embodiment, the homogenization is performed using a rotor and stator.

In another embodiment, a formulation is provided that is prepared by the process of mixing jet-milled or pin-milled belumosudil (in one embodiment, belumosudil mesylate), in a vehicle wherein the vehicle comprises a suspending agent and a thickener in a diluent.

In another embodiment, a liquid formulation comprising belumosudil is provided having components which meet a QTPP having the following requirements: 200 mg belumosudil dose (as free base equivalent), single oral 5 mL dose (dose is expressed as the free base equivalent and is based on the salt correction factor of 1.2124, wherein 200 mg free base is equivalent to 242.48 mg mesylate salt); no taste/mouth feel issues, a uniform color; and sufficient shelf-life for packaging and distribution optionally with refrigeration (in some embodiments, at 2-8° C.).

In another embodiment, a flavor is added to the formulation. In one embodiment, the flavor is added to the liquid formulation when it is prepared, together with other excipients and/or ingredients. In another embodiment, the flavor is stored and/or provided in a separate container and optionally added to the liquid formulation at about the point of administration, as needed and/or desired.

In another embodiment, the flavor is selected from commercially available flavors suitable for use in pharmaceuticals. In some embodiments, the flavor is selected from tropical fruit blend flavor and lemon flavor. In another embodiment, it is tropical fruit blend flavor.

In one embodiment, a method is provided for preparing a liquid formulation comprising belumosudil according to the following steps: (a) a first portion of diluent is dispensed into a suitably sized mixing vessel; (b) a preservative, suspending agent, thickener, and optionally, a sweetener and/or flavor, are dispensed and mixed into the mixing vessel; (c) optionally, a visual inspection is performed after each excipient addition; (d) the mixing vessel is transferred to a homogenizer and the formulation is homogenized; (e) optionally, a visual inspection is performed; (f) a quantity of belumosudil mesylate is added to the homogenized solution and mixed; (g) optionally, a visual inspection is performed; (h) the pH of the formulation is measured; (i) if needed, a pH adjuster is added to the formulation in aliquots and mixed until the desired pH is achieved; and (j) a final portion of diluent is added to produce the belumosudil formulation. Thereafter, the liquid formulation comprising belumosudil may be homogenized and/or allowed to stand until any foam or froth has dissipated and thereafter, it may optionally be visually inspected. The formulation may be dispensed by weight into a final container closure and labelled.

In another embodiment, a liquid formulation comprising belumosudil is provided that is prepared according to the steps set forth in the immediately preceding paragraph.

In another embodiment, a method is provided for preparing a liquid formulation comprising belumosudil according to the following steps: (a) a first portion of sterile water for irrigation is dispensed into a suitably sized mixing vessel; (b) sodium benzoate, sucralose, silica colloidal hydrated and povidone are dispensed and mixed into the mixing vessel; (c) optionally, a visual inspection is performed after each excipient addition; (d) the mixing vessel is transferred to a homogenizer and the formulation is homogenized; (e) optionally, a visual inspection is performed; (f) a quantity of belumosudil mesylate is added to the homogenized solution and mixed; (g) optionally, a visual inspection is performed; (h) the pH of the formulation is measured; (i) tartaric acid is added to the formulation in aliquots and mixed until the desired pH is achieved; and (j) a final portion of sterile water for irrigation is added to produce the belumosudil formulation. Thereafter, the liquid formulation comprising belumosudil may be homogenized and/or allowed to stand until any foam or froth has dissipated and thereafter, it may optionally be visually inspected. The formulation may be dispensed by weight into a final container closure and labelled.

In another embodiment, a liquid formulation comprising belumosudil is provided that is prepared using the steps set forth in the immediately preceding paragraph.

In one embodiment, the foregoing steps are performed using the weight percentages for each excipient and ingredient as set forth in Table 1, Table 2, Table 3, or Table 4.

In another embodiment, a method is provided for preparing a liquid formulation comprising belumosudil according to the following steps: (a) a first portion of purified water is dispensed into a first suitably sized mixing vessel; (b) a quantity of povidone is added to the solution and mixed; (c) optionally, a visual inspection is performed; (d) a quantity of belumosudil mesylate is added to the solution and mixed; (e) optionally, a visual inspection is performed; (f) a second portion of purified water is dispensed into a second suitably sized mixing vessel; (g) sodium benzoate, sucralose, and silica colloidal hydrated are dispensed and mixed into the second mixing vessel; (h) optionally, a visual inspection is performed after each excipient addition; (i) the material from the second mixing vessel is transferred to the first mixing vessel and the formulation is mixed; (j) tartaric acid is added to the formulation in aliquots and mixed until the desired pH is achieved; (k) a final portion of purified water is added; (1) the formulation is mixed; (m) optionally, a visual inspection is performed; and (n). the liquid formulation comprising belumosudil is allowed to stand until any foam or froth has dissipated to produce the belumosudil formulation. Thereafter, it may optionally be visually inspected. The formulation may be dispensed by weight into a final container closure and labelled.

In another embodiment, a liquid formulation comprising belumosudil is provided that is prepared using the steps set forth in the immediately preceding paragraph.

In another embodiment, the mixing steps of the foregoing steps are performed using a paddle mixer. In another embodiment, the mixing steps of the foregoing steps are performed at a rotation speed between about 300 and 600 rpm.

In one embodiment, the foregoing steps are performed using the weight percentages for each excipient and ingredient as set forth in Table 1 or Table 2.

In another embodiment, a method is provided for preparing a liquid formulation comprising belumosudil according to the following steps: (a) a portion of purified water is dispensed into a suitably sized mixing vessel; (b) sodium benzoate, sucralose, silica colloidal hydrated, and povidone are dispensed and mixed into the mixing vessel; (c) a quantity of an about 30% simethicone emulsion is added to the formulation and mixed; (d) a quantity of belumosudil mesylate is added to the solution and mixed; (e) optionally, a visual inspection is performed; (f) tartaric acid is added to the formulation in aliquots and mixed until the desired pH is achieved; (g) a final portion of purified water is added; (h) the formulation is mixed; and (i) optionally, a visual inspection is performed. The formulation may be dispensed by weight into a final container closure and labelled.

In another embodiment, a liquid formulation comprising belumosudil is provided that is prepared using the steps set forth in the immediately preceding paragraph.

In another embodiment, one or more of the mixing steps of the foregoing steps is performed using a homogenizer. In another embodiment, step (h) of the foregoing steps is performed using a homogenizer. In another embodiment, one or more of the mixing steps of the foregoing steps is performed using a Silverson® homogenizer. In another embodiment, one or more of the mixing steps of the foregoing steps is performed using a high shear screen. In another embodiment, one or more of the mixing steps of the foregoing steps is performed at a rotation speed of at least about 1000 rpm. In another embodiment, one or more of the mixing steps of the foregoing steps is performed at a rotation speed between about 4000 and 6000 rpm.

In another embodiment, a method is provided for preparing a liquid formulation comprising belumosudil according to the following steps: (a) a portion of purified water is dispensed into a suitably sized mixing vessel; (b) sodium benzoate is added to the formulation and mixed; (c) sucralose is added to the formulation and mixed; (d) povidone is added to the formulation and mixed; (e) silica colloidal hydrated is added to the formulation and mixed; (f) an about 30% simethicone emulsion is added to the formulation; (g) the formulation is homogenized at 5000 rpm; (h) a quantity of belumosudil mesylate is added to the formulation and mixed; (i) the formulation is homogenized at 5000 rpm using a high shear screen; (j) optionally, a visual inspection is performed; (k) tartaric acid is added to the formulation and mixed until a pH of 3.0 is achieved; (1) a final portion of purified water is added; (m) the formulation is mixed; and (n) optionally, a visual inspection is performed. The formulation may be dispensed by weight into a final container closure and labelled.

In another embodiment, the mixing of the foregoing steps is performed at a rotation speed between about 500 and 600 rpm. In another embodiment, the homogenizing of the foregoing steps is performed using a Silverson® homogenizer. In another embodiment, the homogenizing of the foregoing steps is performed using a high shear screen. In another embodiment, the homogenizing of the foregoing steps is performed at a rotation speed of at least about 1000 rpm. In another embodiment, the homogenizing of the foregoing steps is performed at a rotation speed between about 4000 and 6000 rpm. In another embodiment, the homogenizing of the foregoing steps is performed at a rotation speed of about 5000 rpm.

In one embodiment, the foregoing steps are performed using the weight percentages for each excipient and ingredient as set forth in Table 3 or Table 4.

In another embodiment, the following weight percentages are used to add excipients and ingredients to the formulation: about 2 to 8 weight percentage belumosudil, about 0.02 to 0.08 weight percentage preservative; about 0.1 to 0.4 weight percentage sweetening agent; about 0.2 to 0.8 weight percentage suspending agent; and about 1 to 8 weight percentage thickener.

In another embodiment, the following weight percentages are used to add excipients and ingredients to the formulation: about 2 to 8 weight percentage belumosudil, about 0.02 to 0.08 weight percentage preservative; about 0.1 to 0.4 weight percentage sweetening agent; about 0.2 to 0.8 weight percentage suspending agent; about 2.1 to 8.2 weight percentage thickener; and about 0.25 to 4.0 weight percentage antifoaming agent.

In another embodiment, the following weight percentages are used to add excipients and ingredients to the formulation: (a) for belumosudil, about 2 to 6 weight percentage belumosudil, in another embodiment, about 3 to 5 weight percentage belumosudil (or optionally, about 4.5 to 5 weight percentage belumosudil, or optionally, about 4.75 weight percentage belumosudil); (b) for preservative, about 0.035 to 0.1 weight percentage preservative (or optionally, about 0.05 weight percentage preservative); (c) for sweetening agent, about 0.1 to 0.2 weight percentage sweetening agent (or optionally, about 0.15 weight percentage sweetening agent); (d) for suspending agent, about 0.3 to 1 weight percent suspending agent, optionally, about 0.4 to 0.6 weight percentage suspending agent (or optionally, about 0.5 suspending agent); and (e) for thickener, about 4 to 6 weight percentage thickener (or optionally, about 4.5 to 5.5. weight percentage thickener; or in another embodiment, about 4.9 weight percentage thickener).

In another embodiment, the following weight percentages are used to add excipients and ingredients to the formulation: (a) for belumosudil, about 2 to 6 weight percentage belumosudil, in another embodiment, about 3 to 5 weight percentage belumosudil (or optionally, about 4.5 to 5 weight percentage belumosudil, or optionally, about 4.75 weight percentage belumosudil); (b) for preservative, about 0.035 to 0.1 weight percentage preservative (or optionally, about 0.05 weight percentage preservative); (c) for sweetening agent, about 0.1 to 0.2 weight percentage sweetening agent (or optionally, about 0.15 weight percentage sweetening agent); (d) for suspending agent, about 0.3 to 1 weight percent suspending agent, optionally, about 0.4 to 0.6 weight percentage suspending agent (or optionally, about 0.5 suspending agent); (e) for thickener, about 4 to 6 weight percentage thickener (or optionally, about 4.5 to 5.5. weight percentage thickener; or in another embodiment, about 4.9 weight percentage thickener); and (f) for antifoaming agent, about 0.25 to 4 weight percentage antifoaming agent (or optionally, about 0.5 to 3.5 weight percentage antifoaming agent; or in another embodiment, about 2.0 weight percentage antifoaming agent).

In another embodiment, a liquid formulation comprising belumosudil is provided comprising about 2 to 8 weight percentage belumosudil; in another embodiment, about 2 to 6 weight percentage belumosudil.

In another embodiment, a liquid formulation comprising belumosudil is provided comprising about 0.02 to 0.08 weight percentage sodium benzoate as preservative; in another embodiment, comprising about 0.035 to 0.1 weight percentage sodium benzoate.

In another embodiment, a liquid formulation comprising belumosudil is provided comprising about 0.1 to 0.4 weight percentage sucralose as sweetening agent; in another embodiment, comprising about 0.1 to 0.2 weight percentage sucralose.

In another embodiment, a liquid formulation comprising belumosudil is provided comprising about 0.2 to 0.8 weight percentage colloidal silica as suspending agent; in another embodiment, comprising about 0.4 to 0.6 weight percentage colloidal silica.

In another embodiment, a liquid formulation comprising belumosudil is provided comprising about 1 to 8 weight percentage povidone as thickener. In another embodiment, the liquid formulation comprises about 4 to 6 percent povidone. In certain embodiments, the povidone is povidone 90F.

In another embodiment, a liquid formulation comprising belumosudil is provided comprising about 0.25 to 4 weight percentage of an antifoaming agent. In another embodiment, a liquid formulation comprising belumosudil is provided comprising about 0.25 to 4 weight percentage of an about 30% emulsion of simethicone as an antifoaming agent. In another embodiment, the liquid formulation comprises about 0.5 to 3.5 percent of an about 30% emulsion of simethicone as an antifoaming agent. In another embodiment, the liquid formulation comprises about 2.0 percent of an about 30% emulsion of simethicone as an antifoaming agent. In certain embodiments, the about 30% emulsion of simethicone is Medical Antifoam C Emulsion.

In another embodiment, the belumosudil is pin-milled or jet-milled, prior to addition to the formulation (e.g., per step [f] in the above-described process). In another embodiment jet-milled belumosudil mesylate is added to the formulation.

In another embodiment, a flavor is added to the formulation. In another embodiment, a flavor is added during step (b), together with the preservative, sweetening agent, suspending agent and thickener.

In one embodiment, the liquid formulation of belumosudil is analyzed using a reversed phase gradient HPLC method. In one embodiment, assay testing (e.g., in one embodiment, via HPLC) is performed on the liquid formulation during its manufacture. In one embodiment, assay testing is performed on the first, middle and last bottle filled during the manufacture.

In one embodiment, the liquid formulation comprising belumosudil is formulated to deliver a single dose of 200 mg belumosudil (as free base equivalent) in a volume of about 5 mL. In another embodiment, the liquid belumosudil is formulated to deliver a single dose in the range of 12 to 200 mg (as free base equivalent), in a single volume dose of about 1 to 10 mL, in another embodiment, in a volume of about 5 mL.

In another embodiment, the liquid formulation comprising belumosudil is formulated to deliver a single dose in the range of 10 to 400 mg (as free base equivalent), in a single volume dose of about 1 to 10 mL; in another embodiment, in a single volume dose of about 5 mL.

In another embodiment, the liquid formulation comprising belumosudil is formulated to deliver a single dose of API in the range of about 50 to 200 mg (free base equivalent), in a volume of about 1 to 10 mL, in another embodiment, in a volume of about 5 mL. In another embodiment, the belumosudil is formulated to deliver a dose of API of about 50, 100 or 200 mg (free base equivalent) in a volume of about 1 to 10 mL.

In another embodiment, the liquid formulation comprising belumosudil is prepared to deliver a dose of API of about 50, 100, 200, or 400 mg (as free base equivalent) in a single volume dose of about 5 mL.

In one embodiment, the liquid formulation comprising belumosudil is provided in a bottle containing 10 to 100 mL of the formulation; in another embodiment, the liquid formulation comprising belumosudil is provided in a bottle containing 20 to 60 mL of the liquid formulation. In another embodiment, the liquid formulation comprising belumosudil is provided in a bottle containing about 30 mL+2 mL of the formulation; in some embodiments, the bottle of the liquid formulation comprising belumosudil includes a total of about 5-6 unit doses.

In one embodiment, the liquid formulation comprising belumosudil is provided with a suitably sized oral syringe, and a single unit dose is measured into the syringe prior to dosing. In one embodiment, the syringe is sized to withdraw from 1 to 10 mL of the liquid formulation comprising belumosudil for dosing. In another embodiment, the syringe is sized to withdraw up to 5 mL of the liquid formulation for unit dosing.

In another embodiment, the liquid formulation comprising belumosudil is provided with a suitably sized dosing cup, and a single unit dose is measured into the dosing cup prior to administration. In one embodiment, the dosing cup is sized to hold from 1 to 10 mL of the liquid formulation comprising belumosudil for dosing. In another embodiment, the dosing cup contains a fill line, reflecting the level needed to fill the dosing cup to about 5 mL of the liquid formulation for unit dosing.

In one embodiment, the liquid formulation comprising belumosudil is packaged into a medicinal bottle. In another embodiment, the bottle for packaging the liquid formulation comprising belumosudil contains a child-resistant top or cap. In another embodiment, the liquid formulation comprising belumosudil is packaged into a colored bottle (e.g., amber bottle or blue bottle), and/or is an opaque high-density polyethylene plastic bottle. In one embodiment, the liquid formulation comprising belumosudil is packaged into a suitably sized Type III amber glass container with tamper resistant caps. In one embodiment, the bottle is provided with polyethylene caps. In one embodiment, the liquid formulation comprising belumosudil is packaged into a medicinal bottle containing a polyethylene liner.

In one embodiment, the liquid formulation is refrigerated at a temperature in the range of 2-8° C. In one embodiment, the liquid formulation stored at refrigerated temperature is accompanied by instructions that the formulation should be administered within a specified period of time of when the product is in its final dosage form.

In another embodiment, the liquid formulation is stored at ambient temperature, e.g., of about 15-25° C. In one embodiment, the liquid formulation stored at ambient temperature is accompanied by instructions that the formulation should be administered within a specified period of time of being reconstituted and/or placed in its final form.

In another embodiment, the liquid formulation is accompanied by instructions that once the liquid formulation is pipetted or drawn into a syringe, the formulation should be used within a specified period of time (in one embodiment, within four hours), or discarded.

### Pharmaceutical Compositions, Uses and Dosing

In one aspect, the present invention provides pharmaceutically-acceptable liquid compositions comprising a therapeutically-effective amount of belumosudil formulated together with one or more pharmaceutically-acceptable excipients in a liquid diluent.

In one aspect, the present invention provides pharmaceutically-acceptable liquid compositions comprising a therapeutically-effective amount of belumosudil formulated together with one or more pharmaceutically-acceptable excipients in a liquid diluent at a concentration which is equivalent to about 40 mg/mL belumosudil freebase.

In one aspect, the present invention provides pharmaceutically-acceptable liquid compositions comprising a therapeutically-effective amount of belumosudil formulated together with one or more pharmaceutically-acceptable excipients in a liquid diluent at a concentration which is equivalent to about 40 mg/mL belumosudil freebase, wherein the administrated dose is between about 2.5 and 5.0 mg/kg, particularly about 3.0, 3.5, 4.0, 4.5, and 5.0 mg/kg and more particularly about 4.0 mg/kg. In an embodiment, the administrated dose is about 3.0 mg/kg. In an embodiment, the administrated dose is about 3.5 mg/kg. In an embodiment, the administrated dose is about 4.0 mg/kg. In an embodiment, the administrated dose is about 4.5 mg/kg. In an embodiment, the administrated dose is about 5.0 mg/kg.

In one aspect, the present invention provides pharmaceutically-acceptable liquid compositions comprising a therapeutically-effective amount of belumosudil formulated together with one or more pharmaceutically-acceptable excipients in a liquid diluent at a concentration which is equivalent to about 40 mg/mL belumosudil freebase, wherein the administrated dose is between about 2.5 and 5.0 mg/Kg, particularly about 3.0, 3.5, 4.0, 4.5, and 5.0 mg/kg and more particularly about 4.0 mg/kg, and wherein the maximum administrated dose corresponds to about 200 mg belumosudil freebase once or twice day.

The patient may be an adult or pediatric patient. In an embodiment, the patient is an adult patient. In an embodiment, the patient is a pediatric patient.

In one embodiment, the disclosure provides a method of administering a liquid formulation comprising belumosudil to a patient, including an adult patient, having a body weight of about equal to or greater than 40 kg in the equivalent dosage amount of 200 mg once daily. In another embodiment, the disclosure provides a method of administering a liquid formulation comprising belumosudil to a patient, including an adult patient, having a body weight of about equal to or greater than 40 kg in the equivalent dosage amount of 200 mg twice daily or 400 mg once daily.

In one embodiment, the disclosure provides a method of administering a liquid formulation comprising belumosudil to a pediatric patient having a body weight in the range of about 6 kg to less than 20 kg in the equivalent dosage amount of about 10 to 50 mg administered once daily; in another embodiment, the disclosure provides a method of administering a liquid formulation comprising belumosudil to a pediatric patient having a body weight in the range of about 10 kg to less than 20 kg in the equivalent dosage amount of about 50 mg administered once daily; in another embodiment, in another embodiment, the disclosure provides a method of administering a liquid formulation comprising belumosudil to a pediatric patient having a body weight in the range of about 20 kg to less than 40 kg in the equivalent dosage amount of about 100 mg once daily; and in another embodiment, the disclosure provides a method of administering a liquid formulation comprising belumosudil to a pediatric patient having a body weight of about equal to or greater than 40 kg in the equivalent dosage amount of 200 mg once daily.

In one embodiment, the disclosure provides a method of administering a liquid formulation comprising belumosudil to a patient at a dose between about 2.5 and 5.0 mg/kg, such as about 2.5, 3.0, 3.5, 4.0, 4.5, and 5.0 mg/kg. In an embodiment, the disclosure provides a method of administering a liquid formulation comprising belumosudil to a patient at a dose based on the patient's body weight. In an embodiment, the disclosure provides a method of administering a liquid formulation comprising belumosudil to a patient at a dose of about 2.5 mg/kg; in another embodiment, the disclosure provides a method of administering a liquid formulation comprising belumosudil to a patient at a dose of about 3.0 mg/kg; in another embodiment, the disclosure provides a method of administering a liquid formulation comprising belumosudil to a patient at a dose of about 3.5 mg/kg; in another embodiment, the disclosure provides a method of administering a liquid formulation comprising belumosudil to a patient at a dose of about 4.0 mg/kg; in another embodiment, the disclosure provides a method of administering a liquid formulation comprising belumosudil to a patient at a dose of about 4.5 mg/kg; and in another embodiment, the disclosure provides a method of administering a liquid formulation comprising belumosudil to a patient at a dose of about 5.0 mg/kg. In an embodiment, the dose is administered once daily. In an embodiment, the dose is administered twice daily. In an embodiment, the patient is an adult patient. In an embodiment, the patient is a pediatric patient. In an embodiment, the maximum administrated dose corresponds to about 200 mg belumosudil freebase once or twice day.

The liquid formulations and pharmaceutical compositions disclosed herein may be useful for inhibiting ROCK1 and/or ROCK2 enzymes, preferentially ROCK2, and therefore may be useful in treating diseases regulated by ROCK enzymes such as autoimmune disorders and/or fibrotic disorders including GVHD (chronic and acute), pulmonary fibrosis, idiopathic pulmonary fibrosis, cystic fibrosis, radiation induced fibrosis, or arterial, cardiac, endomyocardial renal, or liver fibrosis; moderate to severe psoriasis, rheumatoid arthritis, multiple sclerosis, systemic lupus erythematosus (SLE), Crohn's disease, dermatitis (e.g., atopic dermatitis), and eczema, among other indications.

The liquid formulations may further be useful in treating bronchiolitis obliterans syndrome (BOS), a potentially severe complication after lung or allogeneic hematopoietic stem cell transplantation (allo-HSCT).

The liquid formulations may further be useful in treating chronic lung allograft dysfunction (CLAD) following lung transplantation.

The following abbreviations may be useful in considering the disclosures herein.

Abbreviations

| | |
|---|---|
| ADI | Acceptable daily limit |
| ADR | Adverse drug reaction |
| AE | Adverse events |
| alloHCT | Allogeneic hematopoietic cell transplantation |
| AUC | Area under the plasma concentration-time curve |
| BID | Twice daily (bi-daily) |
| BM | Bone marrow |
| CDC | Centers for Disease Control and Prevention (U.S.) |
| cGVHD | Chronic graft versus host disease |
| Cmax | Maximum predicted plasma concentration |
| CMC | Carboxymethyl cellulose |
| CMV | Cytomegalovirus |
| Cₒₚₜ | Optical Concentration |
| CR | Complete response |
| DDI | Drug-drug interaction |
| DOR | Duration of response |
| FFS | Failure-free survival |
| HLA | Human leukocyte antigen |
| IMP | Investigational medicinal product |
| IST | Immunosuppressive therapy |
| LSS | Lee Symptom Scale |
| NLT | Not less than |
| NMT | Not more than |
| NT | Not tested |
| ORR | Overall response rate |
| OS | Overall survival |
| PET | Preservative efficacy testing |
| PBPK | Physiologically based pharmacokinetic |
| PBSC | peripheral blood stem cells |
| PK | pharmacokinetics |
| PopPK | Population pharmacokinetic |
| PPI | Proton pump inhibitors |
| PR | Partial response |
| PSD | Particle size distribution |
| PVP | Polyvinylpyrrolidone or povidone |
| QOL | Quality of life |
| RH | Relative humidity |
| RPM | Revolutions per minute |
| RRT | Relative retention time |
| RSD | Relative standard deviation |
| SD | Standard deviation |
| SR | Steroid refractory |
| TEAEs | Treatment-emergent adverse events |
| TTNT | Time to next treatment |
| QD | Daily; every day |
| Q.S. | Quantum satis (sufficient quantity) |
| QTDD | Quality Target Product Profile |

### Examples

### Example 1: Excipients and Preservative Selection

Initially, the excipients listed below in Table 5 were identified for possible inclusion in a belumosudil formulation. In exploring use of these excipients, consideration was given to their suitability for use in pediatric patient populations, for example, through review of legislation relating to age-appropriate excipients for children in the target age group of 3 months to 12 years.

TABLE 5
Excipients Investigated

| Purpose/Function | Excipients Investigated |
|---|---|
| pH adjustment | Citric Acid, Anhydrous |
| | Maleic Acid |
| | Tartaric Acid |
| | Sodium Citrate |
| | Hydrochloric acid |
| Suspending | Xanthan gum (Xantural 75) |
| agent/Glidant | Hydroxyethylcellulose (Natrosol 250HX) |
| | Hypromellose (K4M) |
| | Sodium Carboxymethyl cellulose (Blanose |
| | CMC 7H3SXF) |
| | Silica Colloidal Anhydrous (AEROSIL ®200) |
| | Silicon Dioxide (SYLOID ® 244 FP silica) |
| Wetting agent | Sodium Lauryl Sulphate (SLS)-Kolliphor |
| | SLS Fine |
| Surfactant | Polysorbate 20 (Tween) |
| | Polysorbate 80 (Tween) |
| Sweetening agent | Sodium Saccharin |
| | Sucralose |
| | Acesulfame Potassium |
| Flavor | Banana 501013 Flavor AP0551 |
| | Orange Flavor 501071 AP0551 |
| | Orange Flavor SC611927 |
| | Vanilla Flavor SC750589 |
| | Orange Flexarome Flavor 880021 TFS0504 |
| | Banana Flavor 580033 TP0904 |
| Preservative | Sodium Benzoate |
| | Potassium Sorbate |
| | Sodium methyl hydroxybenzoate |
| | Sodium ethyl hydroxybenzoate |
| | Sodium propyl hydroxybenzoate |
| | Ethyl Parabens |
| Diluent | Sterile water for irrigation |
| | Maltodextrin (Kleptose Linecaps) |
| | Pearlitol 200 SD (Mannitol) |
| | Microcrystalline cellulose |
| Co-solvent | Glycerol |
| Thickener/Binder | Povidone (K30/90) |

All the excipients listed in Table 5 were initially identified as suitable for evaluation. The banana 501013 AP0551 flavor was the only excipient identified as unavailable for use in pediatric formulations on the ground that it includes residual amounts of benzyl alcohol which is considered unacceptable for pediatric populations without sufficient justification.

Certain of the excipients were identified as having specified acceptable daily intake (ADI) levels. In considering the ADI levels, the maximum levels for inclusion in belumosudil pediatric formulations were calculated taking into account the intended maximum daily dose and mean body-weight of a female child of 3 months (5.5 kg), as per the 2000 CDC Growth Charts for the United States (representing the lowest weight for the target age group of 3 months to 12 years).

Compatibility testing for the excipients listed in Table 5 in combination with belumosudil was also performed. Binary mixes of belumosudil and selected excipients were prepared on a 1:1 ratio of belumosudil to excipient, except for binary mixes containing flavor or preservatives that were prepared on a 10:1 ratio. The dry binary mixes were analyzed for appearance and related substances after storage at 2-8° C. (control storage condition) and 40° C./75% RH for 14 days and 28 days. The binary mixes were also tested for visual appearance at initial mixing.

The results from this compatibility study demonstrated that belumosudil is not compatible with hydrochloric acid and maleic acid at 2-8° C. and 40° C./75% RH, over a 14-day period. Thus, hydrochloric acid and maleic acid were not further evaluated for inclusion in the liquid formulation.

The T=14 day excipient compatibility data also showed poor chromatographic resolution between known impurities and belumosudil when in combination with the following preservatives: sodium methyl hydroxybenzoate, sodium ethyl hydroxybenzoate, sodium propyl parabens and ethyl parabens. These preservatives were therefore not further evaluated for use in preparing a liquid formulation.

The T=28 day excipient compatibility data showed a small interaction for the potassium sorbate sample, with an unknown related substance eluting at RRT 0.96, which at 40° C./75% RH had a % area of 0.09%. A pH preservative stability screening study was then performed to identify a suitable target pH, pH modifier and preservative system suitable for inclusion in future formulations to be developed. For example, samples were prepared for evaluation including the following components:
1.1 Belumosudil and water (control);
1.2 Potassium sorbate (no pH modifier);
1.3 Sodium benzoate (no pH modifier);
1.4 Sodium benzoate (adjusted to pH 3 with citric acid);
1.5 Sodium benzoate (adjusted to pH 3 with tartaric acid);
1.6 Sodium benzoate (adjusted with 0.1M citrate buffer to pH 3.0);
1.7 Potassium sorbate (adjusted with 0.1M citrate buffer to pH 5.4); and
1.8 Sodium benzoate (adjusted with 0.1M citrate buffer to pH 4.0).

The above samples (1.1-1.8), were stored at 2-8° C., 25° C./60% RH and 40° C./75% RH for testing at T=0 (visual appearance and pH only), 14 days, and 28 days. Following storage, all samples were assessed for visual appearance, pH and/or related substances and assay.

The results demonstrated that a chemical interaction was observed in the sample 1.2 containing 2.0 mg/mL potassium sorbate and no pH modifier after storage at 40° C./75% RH for 14 days and showed significant increase by 28 days. This pH preservative system was therefore not recommended for inclusion in future formulation prototypes.

Additionally, sample 1.7 (containing potassium sorbate adjusted with 0.1M citrate buffer to pH 5.4), and sample 1.8 (containing sodium benzoate adjusted with 0.1M citrate buffer to pH 4.0), did not easily disperse with manual agitation and manifested significant physical changes on visual observation after storage at 25° C./60% RH and 40° C./75% RH for up to 28 days. These samples (1.7 and 1.8) could not be dispersed by shaking and were therefore not considered during further investigation in developing a belumosudil formulation. All other samples easily dispersed with manual agitation to form a yellow opaque dispersion with yellow foam and were considered suitable for inclusion in future formulation prototypes.

Example 2: Stability Study with pH Modifiers

A pH preservative stability screening study was performed to evaluate the stability of belumosudil in selected aqueous liquid preparations, using selected pH modifiers and preservatives to achieve a pH range 3.0-5.0. Samples were prepared containing sodium benzoate as a preservative and with the pH adjusted with either citric acid or tartaric acid which were compared with samples having no pH modifier. Table 6 describes the samples used in this study.

TABLE 6
Samples Used in pH Stability Study

| | | | | Sample | |
|---|---|---|---|---|---|
| | | Preservative/pH | Timepoint/ | Size and | |
| Sample | Components | Adjusting Agent | Conditions | Quantity | |
| 2.1 | Belumosudil | NA | N/A | ~2 | g |
| | (KD025) | | | | |
| 2.2 | Belumosudil | Sodium benzoate | T = 28 days | ~10 | mL |
| | 48.496 mg/mL | No pH modifier | 40° C./75% RH | | |
| | Dispersion | | | | |
| 2.3 | Belumosudil | Sodium benzoate | T = 28 days | ~10 | mL |
| | 48.496 mg/mL | Adjusted to pH 3.5 | 40° C./75% RH | | |
| | Dispersion | with citric acid | | | |
| 2.4 | Belumosudil | Sodium benzoate | T = 28 days | ~10 | mL |
| | 48.496 mg/mL | Adjusted to pH 3.5 | 40° C./75% RH | | |
| | Dispersion | with tartaric acid | | | |
| 2.5 | Belumosudil | Sodium benzoate, | T = 28 days | ~10 | mL |
| | 48.496 mg/mL | Adjusted to pH 3 | 40° C./75% RH | | |
| | Dispersion | with citrate buffer | | | |
| 2.6 | Belumosudil | Potassium sorbate | T = 28 days | ~10 | mL |
| | 48.496 mg/mL | Adjusted to pH 5.4 | 40° C./75% RH | | |
| | Dispersion | with citrate buffer | | | |

The above samples were prepared for XRPD analysis in three steps: solids were isolated by centrifugation at 3000 rpm for 20 minutes; the supernatant was decanted; and the samples were dried under vacuum at ambient temperature for 18-24 hours overnight. XRPD data of the solid samples was obtained. The XRPD data demonstrated that the API (belumosudil), was stable in the formulation which was a positive indicator for providing a stable formulation. The XRPD data demonstrated that there were consistent additional peaks present for the solids isolated from the sample containing sodium benzoate and no pH modifier and the samples containing sodium benzoate and pH adjusted with either citric acid or tartaric acid which was promising.

However, during the study, a downward drift in pH was observed alongside significant physical changes for samples containing potassium sorbate and citrate buffer. The XRPD data for solid samples obtained from the formulation containing citrate buffer (samples 2.5 and 2.6 in Table 6) demonstrated changes as shown in the XRPD. Based on these observed changes, citrate buffer was not selected for use in the formulation.

Example 3: Pin-Milled Versus Jet-Milled with Povidone

With a suspension formulation, achieving dispersion of API particles uniformly throughout a liquid medium presents a challenge. Use of jet-milled and pin-milled forms of belumosudil in liquid medium were investigated with assessments as to participle size distribution and homogenization of the formulations when initially prepared and over time.

### (a) Comparison of Pin-Milled and Jet-Milled in Water

To investigate pin-milled and jet-milled dispersions of belumosudil in sterile water for irrigation, samples were prepared by weighing a portion of the API into a clear glass bottle and adding sterile water for irrigation to 40 mg/mL.

Samples were assessed using the Sympatec Helos Particle Size Analyzer using the generic method for wet dispersions as outlined below:
R5 Lens and R3 Lens
Sample added until 10-15% Cₒₚₜ achieved
Method run producing 3 replicates (sample stirred and sonicated throughout measurement run)

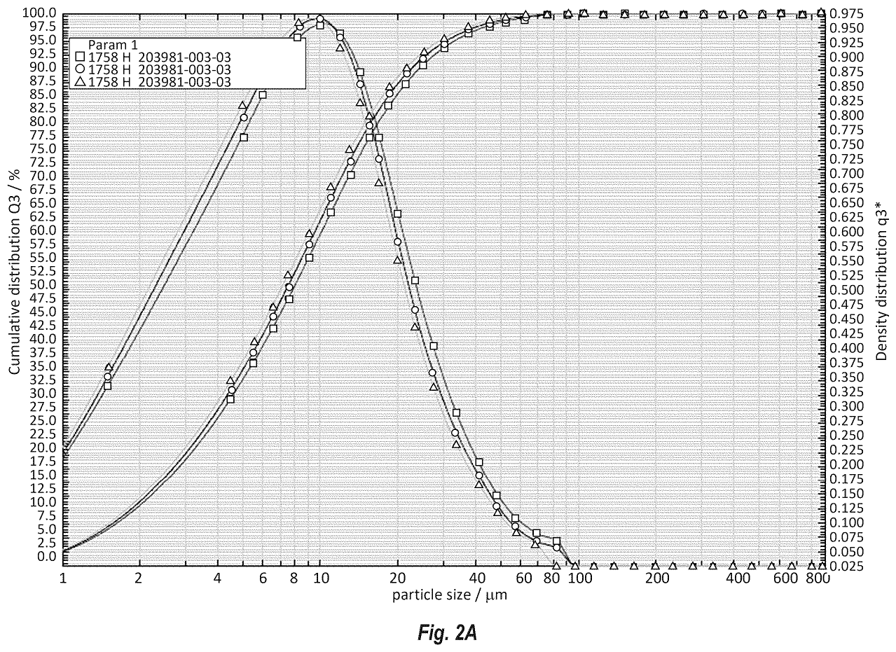

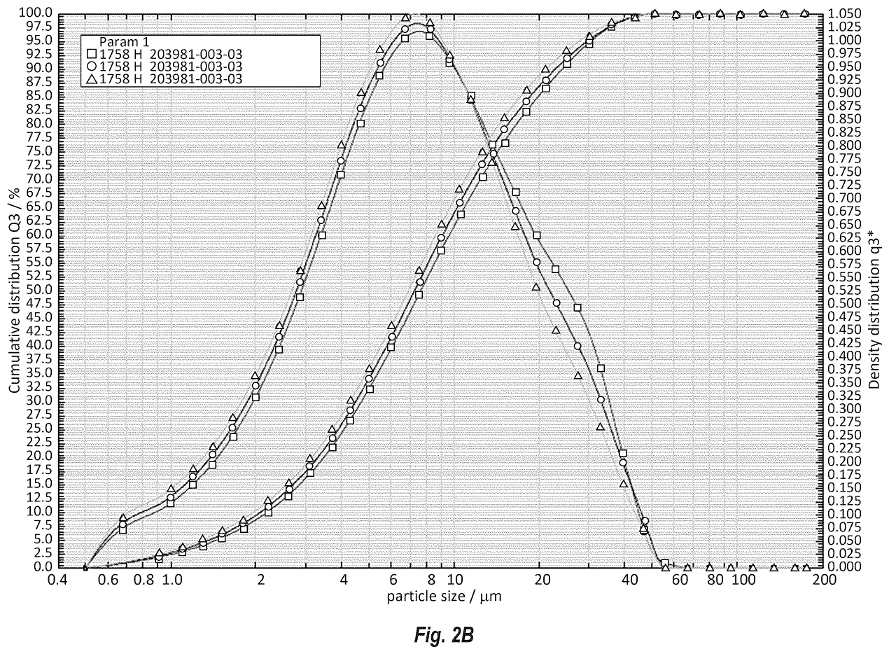

The particle size distribution (PSD) data for the pin-milled API in sterile water for irrigation using the R3 and R5 lenses is presented in FIGS. 2A and 2B, respectively. The results of the pin-milled API show reproducibility between replicates using both the R5 lens and the R3 lens. Upon repeat testing, PSD data was not comparable to the original assessments. There were difficulties obtaining a starting Cₒₚₜ of 10-15% (potentially due to poor dispersion of the API); thus, additional sample was added to achieve the required Cₒₚₜ.

Assessments of the wet dispersion using the jet-milled belumosudil resulted in poor results with readings that were inconsistent between the measurement runs. This was considered as due to poor dispersion of the API. These results reflected that use of jet-milled material may be problematic and limit formulation development. To further use of the jet-milled material, additional hurdles would have to be addressed in terms of stabilizing the formulation and improving dispersion of the jet-milled material.

(b) Comparison of Pin-Milled and Jet-Milled with Various Suspending Agents

To further assess and compare the pin-milled and jet-milled processes in developing a liquid formulation, eleven different formulations were prepared having the composition details as shown below in Tables 5, 6, 7 and 8. In each of Tables 7, 8, 9, and 10, belumosudil mesylate was added at 48.496 mg/mL which is equivalent to 40 mg/mL freebase when adjusted with a 1.2124 salt correction factor.

TABLE 7
Details of Formulations for Pin-milled API (Samples 1A-3A)

| | Sample | | | | | |
|---|---|---|---|---|---|---|
| | 1A | | 2A | | 3A | |
| | | g/50 | | g/50 | | g/50 |
| Components | mg/mL | mL | mg/mL | mL | mg/mL | mL |
| Belumosudil | 48.496 | 2.42 | 48.496 | 2.42 | 48.496 | 2.42 |
| Sodium Benzoate | 0.50 | 0.025 | 0.50 | 0.025 | 0.50 | 0.025 |
| Xanthan gum | n/a | n/a | 3.00 | 0.15 | n/a | n/a |
| (Xantural 75) | | | | | | |
| Hydroxyethylcellulose | n/a | n/a | n/a | n/a | 3.00 | 0.15 |
| (Natrosol 250HX) | | | | | | |
| Hypromellose K4M | n/a | n/a | n/a | n/a | n/a | n/a |
| Sodium Carboxymethyl | n/a | n/a | n/a | n/a | n/a | n/a |
| cellulose (Blanose | | | | | | |
| CMC 7H3SXF) | | | | | | |
| Povidone K30 | n/a | n/a | n/a | n/a | n/a | n/a |
| Tartaric | to | to | to | to | to | to |
| Acid (Q.S.) | pH 3.5 | pH 3.5 | pH 3.5 | pH 3.5 | pH 3.5 | pH 3.5 |
| Sterile Water | 1 | 50 | 1 | 50 | 1 | 50 |
| (To: volume/mL) | | | | | | |

TABLE 8
Details of Formulations for Pin-Milled API (Samples 4A-6A)

| | Sample | | | | | |
|---|---|---|---|---|---|---|
| | 4A | | 5A | | 6A | |
| | | g/50 | | g/50 | | g/50 |
| Components | mg/mL | mL | mg/mL | mL | mg/mL | mL |
| Belumosudil | 48.496 | 2.42 | 48.496 | 2.42 | 48.496 | 2.42 |
| Sodium Benzoate | 0.50 | 0.025 | 0.50 | 0.025 | 0.50 | 0.025 |
| Xanthan gum | n/a | n/a | n/a | n/a | n/a | n/a |
| (Xantural 75) | | | | | | |
| Hydroxyethylcellulose | n/a | n/a | n/a | n/a | n/a | n/a |
| (Natrosol 250HX) | | | | | | |
| Hypromellose K4M | 3.00 | 0.15 | n/a | n/a | n/a | n/a |
| Sodium | n/a | n/a | 3.00 | 0.15 | n/a | n/a |
| Carboxymethyl | | | | | | |
| cellulose | | | | | | |
| Povidone K30 | n/a | n/a | n/a | n/a | 3.00 | 0.15 |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 50 | 1 | 50 | 1 | 50 |
| (To: volume/mL) | | | | | | |

TABLE 9
Details of Formulations for Jet-Milled API (Samples 1B-3B)

| | Sample | | | | | |
|---|---|---|---|---|---|---|
| | 1B | | 2B | | 3B | |
| | | g/50 | | g/50 | | g/50 |
| Components | mg/mL | mL | mg/mL | mL | mg/mL | mL |
| Belumosudil | 48.496 | 2.42 | 48.496 | 2.42 | 48.496 | 2.42 |
| Sodium Benzoate | 0.50 | 0.025 | 0.50 | 0.025 | 0.50 | 0.025 |
| Xanthan gum | n/a | n/a | 3.00 | 0.15 | n/a | n/a |
| (Xantural 75) | | | | | | |
| Hydroxyethylcellulose | n/a | n/a | n/a | n/a | 3.00 | 0.15 |
| (Natrosol 250HX) | | | | | | |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 50 | 1 | 50 | 1 | 50 |
| (To volume/mL) | | | | | | |

TABLE 10
Details of Formulations for Jet-Milled API (Samples 4B-5B)

| | Sample | | | |
|---|---|---|---|---|
| | 4B | | 5B | |
| | (3 mg/mL Hypromellose K4M) | | (3 mg/mL Povidone K30) | |
| Components | mg/mL | g/50 mL | mg/mL | g/50 mL |
| Belumosudil | 48.496 | 2.42 | 48.496 | 2.42 |
| (jet-milled) | | | | |
| Sodium Benzoate | 0.50 | 0.025 | 0.50 | 0.025 |
| Hypromellose K4M | 3.00 | 0.15 | n/a | n/a |
| Povidone K30 | n/a | n/a | 3.00 | 0.15 |
| Tartaric Acid | to pH 3.5 | to pH 3.5 | to pH 3.5 | to pH 3.5 |
| (Q.S.) | | | | |
| Sterile Water | 1 | 50 | 1 | 50 |
| (To: volume/mL) | | | | |

In preparing the formulations described in Tables7, 8, 9, and 10, the following steps were used:
1. to a pre-calibrated beaker, about 40 mL of sterile water was added for irrigation;
2. while stirring, sodium benzoate was added and mixed until dissolved;
3. suspending agent was added and mixed until a uniform mixture was seen;
4. while mixing, belumosudil was added and mixed until a uniform mixture was seen;
5. the pH was measured and if required, tartaric acid was added in aliquots until pH 3.5±0.2 was achieved;
6. sterile water was added to make up to volume for irrigation and the pH was recorded; and
7. the liquid formulation comprising belumosudil was transferred to a 60 mL clear glass bottle.

The samples were assessed for ease of dispersion of the API, levels of sedimentation and ease of re-dispersing any sediment.

The API dispersed easily for samples 1A (pin-milled) (Table 7), and 1B (jet-milled) (Table 9), with no suspending agent. After about 24 hours, a small amount of flocculation was observed but this easily resuspended with shaking. Both the pin-milled and jet-milled API foamed when shaken, but the foam was less apparent with the jet-milled API (sample G), with only a small upper layer of foam after about 30 seconds.

Conversely, the API did not easily disperse in batches containing xanthan gum (Xantural 75) (i.e., samples 2A and 2B/Tables 7 and Table 9, respectively). These samples required homogenization to aid dispersion of the API and both batches did not appear as a homogenous formulation even following homogenization. From this observation, it was concluded that xanthan gum is not an ideal suspending agent. Sample 5A (Table 8), containing sodium carboxymethyl cellulose showed severe flocculation, and it was difficult to disperse the API, even with homogenization. A large amount of residue was also observed at the bottom of the glass beaker after transferring the contents of the glass beaker to a glass bottle.

For other samples using pin-milled and jet-milled and hydroxyethylcellulose-Natrosol 250HX) (samples 3A and 3B); hypromellose K4M (samples 4A and 4B), and Povidone K30 (samples 6A and 5B), the API easily dispersed using a magnetic stirrer to form a yellow opaque suspension, with no differences observed between the batches. Small aggregates of undispersed material were observed as residue when transferring from beaker to bottle suggesting that homogenization may still be required. Overall, it was observed that use of povidone provided improved results.

For both samples 4B and 5B (Table 10), containing jet-milled API, the API easily dispersed using just a magnetic stirrer to form a yellow opaque suspension. There were no differences observed between the samples. These observations were consistent with those previously noted for equivalent batches incorporating the pin-milled API (i.e., samples 4A and 6A [Table 8], containing hypromellose K4M and povidone K30, respectively).

All samples in Tables 7, 8, 9, and 10 were monitored over 4 days and 2 weeks at ambient temperature and assessed for visual appearance. Samples 4B and 5B (Table 10), were manufactured approximately 1 week after all other samples and so were only assessed at T=4 days. Results are outlined in Table 11 (pin-milled API) and Table 12 (jet-milled API).

TABLE 11
Visual Observations After 4 Days and 2 Weeks of Pin-Milled Formulations

| | 1A | 2A | 3A | 4A | 5A | 6A |
|---|---|---|---|---|---|---|
| | (No | (3.0 mg/mL | (3.0 mg/mL | (3.0 mg/mL | (3.0 mg/mL | (3.0 mg/mL |
| | suspending | Xantural | Natrosol | Hypromellose | Blanose | Povidone |
| | agent) | 75) | 250HX) | K4M) | CMC) | K30) |
| Initial | Yellow | Yellow | Yellow | Yellow | Significant | Yellow |
| | opaque | opaque | opaque | opaque | degree of | opaque |
| | suspension, | suspension, | suspension, | suspension, | flocculation | suspension, |
| | API | API did not | API | API | of API | API |
| | visually | appear | visually | visually | and | visually |
| | dispersed | homogenously | dispersed | dispersed | separation | dispersed |
| | | dispersed | | | | |
| 4 | Yellow | Yellow | Yellow | Yellow | Significant | Yellow |
| days | opaque | opaque | opaque | opaque | degree of | opaque |
| | suspension, | suspension, | solid | suspension, | flocculation | suspension, |
| | flocculation | flocculation | structure | API | of API | API |
| | of API | of API | (did not | visually | and | visually |
| | observed | observed | pour on | dispersed | separation | dispersed |
| | | | inversion) | | | |
| ~2 | Yellow | Yellow | Yellow | Yellow | Significant | Yellow |
| weeks | opaque | opaque | opaque | opaque | degree of | opaque |
| | suspension, | suspension, | solid | suspension, | flocculation | suspension, |
| | flocculation | flocculation | structure | API | | API |
| | of API | of API | (did not | visually | | visually |
| | observed. | observed | pour on | dispersed* | | dispersed |
| | Separation | | inversion) | | | |
| | with solid | | | | | |
| | gel-like | | | | | |
| | structure in | | | | | |
| | clear liquid | | | | | |

*After 20 days at ambient temperature, sample transformed to a solid gel-like formulation.

TABLE 12
Visual Observations After 4 Days and 2 Weeks of Jet-Milled Formulations

| | 1B | | | 4B | |
|---|---|---|---|---|---|
| | (No | 2B | 3B | (3.0 mg/mL | 5B |
| | suspending | (3.0 mg/mL | (3.0 mg/mL | Hypromellose | (3.0 mg/mL |
| | agent) | xantural 75) | Natrosol 250HX) | K4M) | Povidone K30) |
| Initial | Yellow opaque | Yellow opaque | Yellow opaque | Yellow opaque | Yellow opaque |
| | suspension, | suspension, | suspension, API | suspension, | suspension, |
| | API visually | API did not | visually dispersed | API visually | API visually |
| | dispersed | appear | (No photo | dispersed | dispersed |
| | | homogenously | available) | | |
| | | dispersed | | | |
| 4 days | Yellow opaque | Separation | Yellow opaque | Yellow opaque | Yellow opaque |
| | suspension, | observed with | solid structure | suspension, | suspension, |
| | API appeared | solid yellow | (did not pour on | API visually | API visually |
| | visually | structure and | inversion | dispersed | dispersed |
| | dispersed | clear liquid | | | |
| | | layer | | | |
| ~2 | Yellow opaque | Yellow opaque | Yellow opaque | Not Tested | Not Tested |
| weeks | suspension, | suspension, | solid structure | | |
| | flocculation of | flocculation of | (did not pour on | | |
| | API observed | API observed | inversion) | | |

The observations demonstrated that samples containing either no suspending agent, Natrosol 250HX, Xantural 75 or sodium carboxymethyl cellulose (manufactured with both jet-milled API and pin-milled API), all showed various signs of either separation, flocculation or solidification. Although samples incorporating hypromellose K4M (samples 4A and 4B), were observed as a yellow opaque suspension with the API visually dispersed after storage at ambient temperature for 4 days and/or 2 weeks, sample 4A (3 mg/mL hypromellose K4M with pin-milled API) had become a solid gel-like material when observed after 20 days at ambient temperature. When shaking to liquefy and redisperse, the API began aggregating in the formulation. Accordingly, it was determined that hypromellose K4M was not an optimal choice for further development.

Samples using povidone K30 (manufactured with both jet-milled API and pin-milled API) remained a yellow opaque suspension with the API visually dispersed after storage at ambient temperature for 4 days, also after about 2 weeks. From this study, povidone K30 was considered the leading suspending agent for further evaluation.

### Example 4: Particle Size Distribution Analysis

Based on the results from the visual observation analyses in Example 3, four of the liquid formulations comprising belumosudil were selected for further assessment as to their particle size distribution. Measurements were carried out on the following four formulations: sample 3A (pin-milled formulation with hydroxyethylcellulose); sample 3B (jet-milled formulation with hydroxyethylcellulose); sample 4A (pin-milled formulation with Hypromellose K4M); and sample 6A (pin-milled formulation with povidone).

Samples were assessed using the Sympatec Helos Particle Size Analyzer, with sample added until 10-15% Cₒₚₜ was achieved. Two different lens assemblies were used giving the following ranges for particle size assessment, R3 Lens (0.5-175 μm) and the R5 Lens (4.8-875 μm). Three replicates were performed as part of the measurement run, with the sample stirred and sonicated as part of the method.

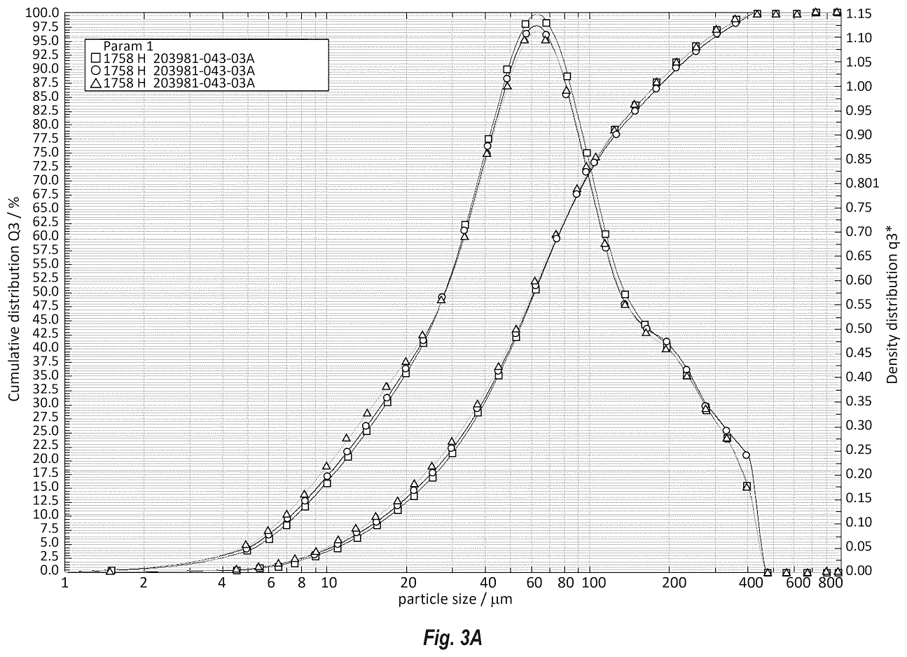

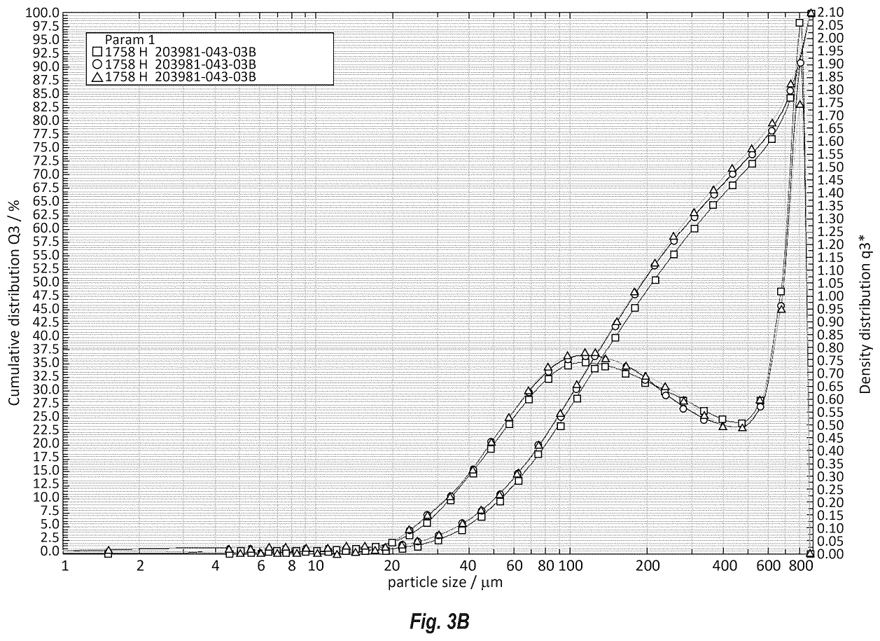

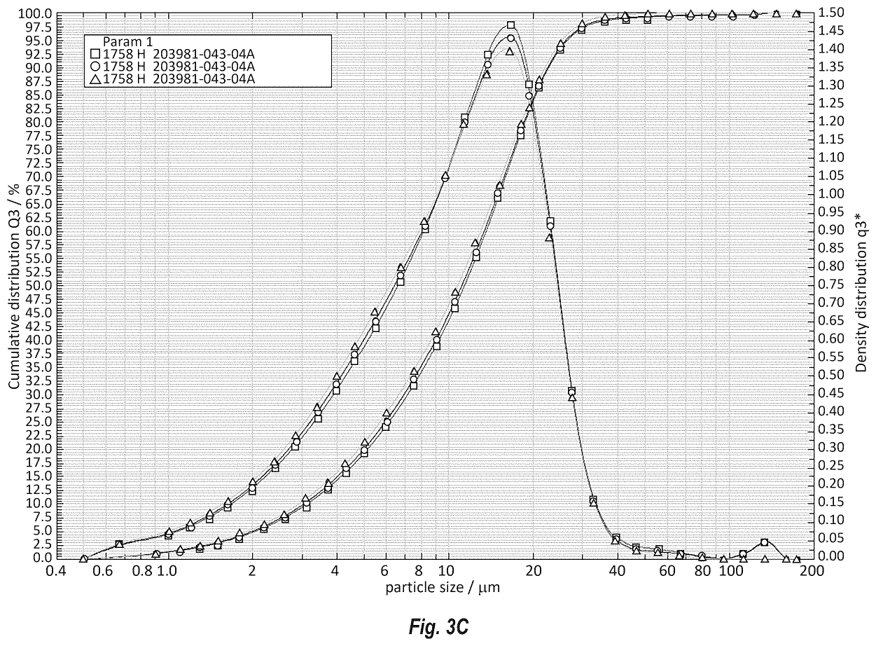

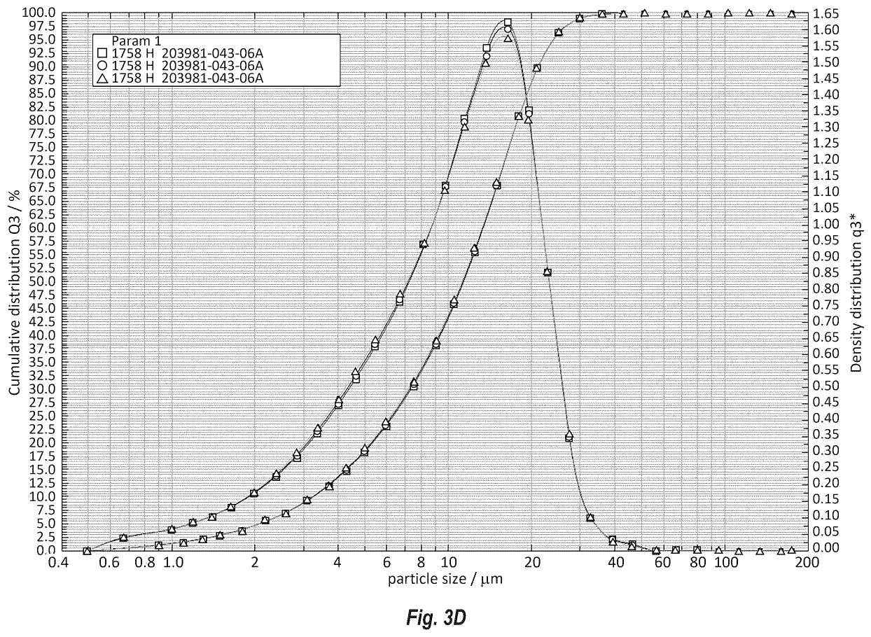

For samples 3A and 3B, assessments were made using the R5 lens. Results are shown in FIG. 3A (sample 3A) and 3B (sample 3B). The results demonstrate that consistent replicates were obtained for both batches, although the particle size recorded was larger than expected (when compared to the dry PSD of milled API). Upon investigation it was noted that the formulations containing Natrosol 250HX formed solid aggregates on dilution with water, rather than dispersing.

This could be a result of severe flocculation which would be consistent with the PSD measurement giving unexpectedly high values, comparing both API samples this effect is particularly worse with the jet-milled sample (sample 3B).

For sample 4A (pin-milled Hypromellose K4M formulation), PSD was assessed using both R5 and R3 lens. The most consistent replicates were obtained when using the R3 lens, and the results are shown in FIG. 3C. A number of peaks at larger sizes were observed and considered to be due to air bubbles. The stirrer speed was reduced from 80% to 50% to minimize aeration and generation of air bubbles to achieve consistent replicates.

For sample 6A (pin-milled formulation with povidone), the sample was assessed using both R5 and R3 lens. As with sample 4A, the most consistent replicates were obtained when using the R3 lens; the results are shown in FIG. 3D. The flocculation/aggregates that were present in samples 3A and 3B with Natrosol 250HX were not observed for this formulation.

The D10, D50 and D90 values for sample 6A were consistent with the values for sample 4A, both of which were manufactured using the pin-milled Belumosudil. The results provided confidence that the method was able to produce accurate and consistent results on these two formulation prototypes.

In summary, when testing samples 3A and 3B (containing hydroxyethylcellulose/Natrosol 250HX), aggregates were observed when diluting in water in the mixing tank leading to inconsistent data between replicates. The results were therefore not representative of the expected particle size distribution of the API. Differences were observed between the pin-milled and jet-milled API, with larger aggregates and flocculation observed with sample 3B which was manufactured using jet-milled API, as compared with sample 3A (using pin-milled). This observation confirmed the conclusions from Example 3 that the jet-milled material may be problematic and present hurdles in formulation development.

Consistent replicates were obtained when analyzing samples 4A and 6A (pin-milled formulations with Hypromellose K4M and povidone K30), when using the R3 lens and after reducing the stirring speed from 80% to 50%. Occasional peaks at larger values were observed which were attributed to air bubbles. Results were similar for both samples 4A and 6A. This suggests that these formulation prototypes allowed for more consistent PSD results compared to the particle size assessment of wet dispersion of just API in water as shown in FIGS. 2A and 2B and discussed above in Example 3 (section (a)). From this assessment, pin-milled methods were considered suitable for developing liquid formulations comprising belumosudil.

### Example 5: Xanthan Gum & Sodium CMC Thickener System Investigation

In Example 3, it was shown that samples containing 3 mg/mL xanthan gum (Xantural 75) (Sample 2A) and 3 mg/mL Sodium CMC (Blanose CMC 7H3SXF) (Sample 5A) showed severe flocculation and difficulty in dispersing the API. In this experiment, four additional formulations were investigated to determine whether the flocculation could be addressed via the following:
Adding the suspending agent after the API is fully dispersed;
Reducing the level of xanthan gum and sodium CMC;
Including silica products (i.e., AEROSIL® 200 and SYLOID® 244 FP) to prevent caking and aid resuspension.

Samples 7A, 8A, 9A and 10A were manufactured on a 50 mL batch size according to the composition details presented in Table 13.

The formulation preparation was planned according to the following steps: (1) to a suitable beaker, about 40 mL of sterile water was added; (2) while mixing using a magnetic stirrer, sodium benzoate was added and mixed until dissolved; (3) while mixing, belumosudil (pin-milled) was added and mixed for 15 minutes and until a uniform dispersion was obtained; (4) while mixing, Xantural 75 or Blanose CMC 7H3SXF were added and mixed for 30 minutes and until uniform; (5) the pH was measured and if required, tartaric acid was added to adjust to pH 3.5±0.2; and (6) sterile water was added to make up to volume and the formulation was mixed for 15 minutes and until uniform.

TABLE 13
Formulations for Xanthan Gum and Sodium CMC Experiments*

| | Sample | | | | | | | |
|---|---|---|---|---|---|---|---|---|
| | 7A | | 8A | | 9A | | 10A | |
| | | g/50 | | g/50 | | g/50 | | g/50 |
| Component | mg/mL | mL | mg/mL | mL | mg/mL | mL | mg/mL | mL |
| Belumosudil | 48.496* | 2.42 | 48.496* | 2.42 | 48.496* | 2.42 | 48.496* | 2.42 |
| (Pin-milled) | | | | | | | | |
| Sodium Benzoate | 0.50 | 0.025 | 0.50 | 0.025 | 0.50 | 0.025 | 0.50 | 0.025 |
| Xanthan Gum | 1.50 | 0.075 | 3.00 | 0.15 | n/a | n/a | n/a | n/a |
| (Xantural 75) | | | | | | | | |
| Sodium CMC | n/a | n/a | n/a | n/a | 3.00 | 0.15 | 1.50 | 0.075 |
| Tartaric Acid | to | to | to | to | to | to | to | to |
| (Q.S.) | pH 3.5 | pH 3.5 | pH 3.5 | pH 3.5 | pH 3.5 | pH 3.5 | pH 3.5 | pH 3.5 |
| Sterile Water | 1 | 50 | 1 | 50 | 1 | 50 | 1 | 50 |
| (To: volume/mL) | | | | | | | | |

*48.496 mg/mL equivalent to 40 mg/mL adjusted with 1.2124 correction factor.

However, all samples (7A, 8A, 9A and 10A) showed signs of flocculation, as seen previously in Example 3, after addition of the API (step 4). Samples 7A and 8A containing xanthan gum were homogenized using the Silverson homogenizer fitted with the general-purpose disintegrating head for ~1 minute. The severe flocculation previously seen was still observed. Due to the severe flocculation, manufacture was halted following step 4.

This experiment demonstrated that changing the order of addition did not prevent the flocculated material as seen previously. Reducing the suspending agents did show some improvement, but flocculation and thickening still occurred and manufacture was halted for all four formulations as a result. Based on these observations, xanthan gum and sodium CMC were ruled out as suitable suspending agents for use in a belumosudil pharmaceutical formulation.

### Example 6: Povidone Suspending Agent System Experiments

Additional formulations as summarized below were manufactured incorporating various levels of povidone K30 as the lead suspending agent. AEROSIL® 200 and SYLOID® 244 FP were incorporated into these formulations to assess their impact on preventing caking. Each formulation was manufactured on a 100 mL batch size using both pin-milled API and jet-milled API to allow for a comparative assessment of the two. Composition details are shown in Tables 14A and 14B (pin-milled API), and Tables 15A and 15B (jet-milled API). In each of Tables 14A, 14B, 15A and 15B, belumosudil mesylate was added at 48.496 mg/mL which is equivalent to 40 mg/mL freebase when adjusted with a 1.2124 salt correction factor.

TABLE 14A
Pin-Milled Formulations for Povidone Study (Samples 11A-13A)

| | Sample | | | | | |
|---|---|---|---|---|---|---|
| | 11A | | 12A | | 13A | |
| | | g/100 | | g/100 | | g/100 |
| Component | mg/mL | mL | mg/mL | mL | mg/mL | mL |
| Belumosudil | 48.496 | 4.8496 | 48.496 | 4.8496 | 48.496 | 4.8496 |
| Sodium Benzoate | 0.50 | 0.05 | 0.50 | 0.05 | 0.50 | 0.05 |
| Povidone K30 | 3.00 | 0.30 | 6.00 | 0.60 | 9.00 | 0.90 |
| AEROSIL ® 200 | n/a | n/a | n/a | n/a | n/a | n/a |
| SYLOID ®244 FP | n/a | n/a | n/a | n/a | n/a | n/a |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 100 | 1 | 100 | 1 | 100 |
| (To: volume/mL) | | | | | | |

TABLE 14B
Pin-Milled Formulations for Povidone Study (Samples 14A-16A)

| | Sample | | | | | |
|---|---|---|---|---|---|---|
| | 14A | | 15A | | 16A* | |
| | | g/100 | | g/100 | | g/100 |
| Component | mg/mL | mL | mg/mL | mL | mg/mL | mL |
| Belumosudil | 48.496 | 4.8496 | 48.496 | 4.8496 | 48.496 | 4.8496 |
| Sodium Benzoate | 0.50 | 0.05 | 0.50 | 0.05 | 0.50 | 0.05 |
| Povidone K30 | 6.00 | 0.60 | 6.00 | 0.60 | n/a | n/a |
| AEROSIL ® 200 | 5.00 | 0.50 | n/a | n/a | n/a | n/a |
| SYLOID ®244 FP | n/a | n/a | 5.00 | 0.50 | n/a | n/a |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 100 | 1 | 100 | 1 | 100 |
| (To: volume/mL) | | | | | | |

TABLE 15A
Jet-Milled Formulations for Povidone Study (Samples 11B-13B)

| | Sample | | | | | |
|---|---|---|---|---|---|---|
| | 11B | | 12B | | 13B | |
| Component | mg/mL | g/100 mL | mg/mL | g/100 mL | mg/mL | g/100 mL |
| Belumosudil | 48.496 | 4.8496 | 48.496 | 4.8496 | 48.496 | 4.8496 |
| Sodium Benzoate | 0.50 | 0.05 | 0.50 | 0.05 | 0.50 | 0.05 |
| Povidone K30 | 3.00 | 0.30 | 6.00 | 0.60 | 9.00 | 0.90 |
| AEROSIL ®200 | n/a | n/a | n/a | n/a | n/a | n/a |
| SYLOID ®244 FP | n/a | n/a | n/a | n/a | n/a | n/a |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 100 | 1 | 100 | 1 | 100 |
| (To: volume/mL) | | | | | | |

TABLE 15B
Jet-Milled Formulations for Povidone Study (Samples 14B-16B)

| | Sample | | | | | |
|---|---|---|---|---|---|---|
| | 14B | | 15B | | 16B* | |
| Component | mg/mL | g/100 mL | mg/mL | g/100 mL | mg/mL | g/100 mL |
| Belumosudil | 48.496 | 4.8496 | 48.496 | 4.8496 | 48.496 | 4.8496 |
| Sodium Benzoate | 0.50 | 0.05 | 0.50 | 0.05 | 0.50 | 0.05 |
| Povidone K30 | 6.00 | 0.60 | 6.00 | 0.60 | n/a | n/a |
| AEROSIL ®200 | 5.00 | 0.50 | n/a | n/a | n/a | n/a |
| SYLOID ®244 FP | n/a | n/a | 5.00 | 0.50 | n/a | n/a |
| Tartaric Acid | to | to | to | to | to | to |
| (Q.S.) | pH 3.5 | pH 3.5 | pH 3.5 | pH 3.5 | pH 3.5 | pH 3.5 |
| Sterile Water | 1 | 100 | 1 | 100 | 1 | 100 |
| (To: volume/mL) | | | | | | |

The formulations in Tables 14A-14B and 15A-15B were prepared with the following steps:
1. To a suitable beaker, about 80% of the total quantity of sterile water was added for irrigation;
2. While mixing using a magnetic stirrer, sodium benzoate was added and mixed until dissolved;
3. While mixing, povidone K30 was added and mixed for 30 minutes and until uniform;
4. While mixing, belumosudil (pin or jet-milled) was added and mixed for 15 minutes and until a uniform dispersion was obtained;
5. Measure the pH. If required use tartaric acid to adjust to pH 3.5±0.2
6. Make up to volume using sterile water for irrigation and mix for 15 minutes and until uniform.

A 12 mL portion of each formulation was transferred into a 15 mL plastic centrifuge tube to test for the rate of sedimentation. Separate 30 mL portions of each of formulation were also transferred into 60 mL glass bottles to be stored for 7 days at ambient temperature and 50° C. After storage, all samples were assessed for visual appearance, level of sedimentation, ease of resuspension and pH.

### (a) Initial Sedimentation and Appearance Testing

The visual appearance and levels of sediment in the samples in 15 mL centrifuge tubes stored at ambient temperature were recorded at the following time points; 1 hour, 2 hours and 24 hours, 4 days and 7 days. Results are presented in Table 16 (pin-milled formulations), and Table 17 (jet-milled formulation). In Table 16, the “loosely packed” material could only be seen by shining a light through the sample.

TABLE 16
Sediment Levels Over Time (Pin-milled) With Varied Povidone Levels

| | Sample | | | | | |
|---|---|---|---|---|---|---|
| | 11A | 12A | 13A | 14A | 15A | 16A |
| | Details | | | | | |
| | | | | 6 mg/mL | 3 mg/mL | |
| | | | | Povidone | Povidone | No |
| | 3 mg/mL | 6 mg/mL | 9 mg/mL | K30 + | K30 + | suspending |
| | Povidone | Povidone | Povidone | 5 mg/mL | 5 mg/mL | agent, |
| | K30 | K30 | K30 | Aerosil 200 | Syloid 244FP | Control |
| Timepoint | Sediment | | | | | |
| Initial | None | None | None | None | None | None |
| 15 minutes | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| 30 minutes | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| 1 hour | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| 2 hours | <0.1 mL | <0.1 mL | <0.1 mL | <0.1 mL | <0.1 mL | *See |
| | hard | hard | hard | hard | hard | Note 1 |
| | sediment + | sediment + | sediment + | sediment + | sediment + | |
| | 2.25 mL | 2.75 mL | 3.25 mL | 4.5 mL | 4.5 mL | |
| | loosely | loosely | loosely | loosely | loosely | |
| | packed | packed | packed | packed | packed | |
| 24 hours | <0.1 mL | <0.1 mL | <0.1 mL | <0.1 mL | <0.1 mL | **See |
| | hard | hard | hard | hard | hard | Note 2 |
| | sediment + | sediment + | sediment + | sediment + | sediment + | |
| | 2.0 mL | 2.25 mL | 2.75 mL | 3.5 mL | 3.75 mL | |
| | loosely | loosely | loosely | loosely | loosely | |
| | packed | packed | packed | packed | packed | |
| 4 days | <0.1 mL | <0.1 mL | <0.1 mL | <0.1 mL | <0.1 mL | **See |
| | hard | hard | hard | hard | hard | Note 2 |
| | sediment + | sediment + | sediment + | sediment + | sediment + | |
| | 2.0 mL | 2.25 mL | 2.75 mL | 3.5 mL | 3.5 mL | |
| | loosely | loosely | loosely | loosely | loosely | |
| | packed | packed | packed | packed | packed | |
| 7 days | <0.1 mL | <0.1 mL | <0.1 mL | <0.1 mL | <0.1 mL | **See |
| | hard | hard | hard | hard | hard | Note 2 |
| | sediment + | sediment + | sediment + | sediment + | sediment + | |
| | 1.75 mL | 2.25 mL | 2.75 mL | 3.5 mL | 3.5 mL | |
| | loosely | loosely | loosely | loosely | loosely | |
| | packed | packed | packed | packed | packed | |
| | Disperses | Disperses | Disperses | Disperses | Disperses | |
| | in 25 | in 22 | in 17 | in 15 | in 7 | |
| | inversions | inversions | inversions | inversions | inversions | |

*Note 1:
Sample beginning to thicken; noticeable on gentle tilting,
**Note 2:
Sample thickened considerable; noticed on gentle tilting.

TABLE 17
Sediment Levels Over Time (Jet-milled) With Varied Povidone Levels

| | Sample | | | | | |
|---|---|---|---|---|---|---|
| | 11B | 12B | 13B | 14B | 15B | 16B |
| | Details | | | | | |
| | | | | 6 mg/mL | 3 mg/mL | |
| | | | | Povidone | Povidone | No |
| | 3 mg/mL | 6 mg/mL | 9 mg/mL | K30 + | K30 + | suspending |
| | Povidone | Povidone | Povidone | 5 mg/mL | 5 mg/mL | agent, |
| | K30 | K30 | K30 | Aerosil 200 | Syloid 244FP | Control |
| Timepoint | Sediment/Supernatant | | | | | |
| Initial | None | None | None | None | None | None |
| 15 minutes | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| | 0.3 mL | 0.25 mL | 0.2 mL | *See | *See | *See |
| | second | second | second | Note 1 | Note 1 | Note 1 |
| | layer | layer | layer | | | |
| | *See | *See | *See | | | |
| | Note 1 | Note 1 | Note 1 | | | |
| 30 minutes | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| | 0.35 mL | 0.25 mL | 0.2 mL | *See | 0.3 mL | *See |
| | second | second | second | Note 2 | second | Note 1 |
| | layer | layer | layer | | layer | |
| | *See | *See | *See | | *See | |
| | Note 1 | Note 1 | Note 1 | | Note 1 | |
| 1 hour | <0.1 mL | <0.1 mL | <0.1 mL | <0.1 mL | <0.1 mL | <0.1 mL |
| | hard | hard | hard | hard | hard | hard |
| | sediment | sediment | sediment | sediment | sediment | sediment |
| | 0.5 ml | 0.4 mL | 0.3 mL | *See | 0.55 mL | *See |
| | second | second | second | Note 2 | second | Note 1 |
| | layer | layer | layer | | layer | |
| | *See | *See | *See | | *See | |
| | Note 1 | Note 1 | Note 1 | | Note 1 | |
| 2 hours | <0.1 mL | <0.1 mL | <0.1 mL | <0.1 mL | <0.1 mL | ***See |
| | hard | hard | hard | hard | hard | Note 3 |
| | sediment | sediment | sediment | sediment | sediment | |
| | 0.5 ml | 0.4 mL | 0.3 mL | *See | 0.55 mL | |
| | second | second | second | Note 2 | second | |
| | layer | layer | layer | | layer | |
| | *See | *See | *See | | *See | |
| | Note 1 | Note 1 | Note 1 | | Note 1 | |
| 24 hours | <0.1 mL | <0.1 mL | <0.1 mL | <0.1 mL | <0.1 mL | ***See |
| | hard | hard | hard | hard | hard | Note 3 |
| | sediment | sediment | sediment | sediment | sediment | |
| | 0.8 ml | 0.6 mL | 0.5 mL | *See | *See | |
| | second | second | second | Note 2 | Note 2 | |
| | layer | layer | layer | | | |
| | *See | *See | *See | | | |
| | Note 1 | Note 1 | Note 1 | | | |
| 4 days | <0.1 mL | <0.1 mL | <0.1 mL | <0.1 mL | <0.1 mL | ***See |
| | hard | hard | hard | hard | hard | Note 3 |
| | sediment | sediment | sediment | sediment | sediment | |
| | 1.5 ml | 0.8 mL | 0.6 mL | *See | *See | |
| | second | second | second | Note 2 | Note 2 | |
| | layer | layer | layer | | | |
| | *See | *See | *See | | | |
| | Note 1 | Note 1 | Note 1 | | | |
| 7 days | <0.1 mL | <0.1 mL | <0.1 mL | <0.1 mL | <0.1 mL | ***See |
| | hard | hard | hard | hard | hard | Note 3 |
| | sediment | sediment | sediment | sediment | sediment | |
| | 1.5 ml | 1.0 mL | 1.0 mL | 2.0 mL | 2.0 mL | |
| | second | second | second | second | second | |
| | layer | layer | layer | layer | layer | |
| | Disperses | Disperses | Disperses | Disperses | Disperses | |
| | in 25 | in 20 | in 17 | in 17 | in 10 | |
| | inversions | inversions | inversions | inversions | inversions | |

*Note 1:
Signs of solid material adhered to centrifuge wall.
**Note 2:
Large amount of solid material adhered to centrifuge wall; difficult to determine amount of sediment
***Note 3:
Sample thickened and became solid-like. Could be held upside down and remained a gel like material.

As reflected in Tables 16 and 17, differences in behavior of the sedimentation were observed between samples containing pin-milled API and jet-milled API. For the pin-milled API, as the level of povidone K30 was increased, the amount of settled material also increased. The inclusion of AEROSIL® 200 and SYLOID® 244 FP appeared to reduce the rate of settling. The appeared to outperform the AEROSIL®. For samples containing jet-milled API, the second layer of sediment was reduced as levels of povidon K30 were increased and with the addition of AEROSIL® 200 or SYLOID® 244 FP.

From this experiment, the important observation was made that including colloidal silica was beneficial in preventing caking and easing redispersion, with samples containing SYLOID® 244FP requiring the fewest number of inversions to fully redisperse the API.

The visual appearance of the samples stored at ambient temperature in 30 mL glass bottles was also recorded after 24 hours and after 7 days at ambient temperature and at 50° C. over 7 days. The elevated (50° C.) temperature was used to accelerate conditions and stress the formulation. The “% sediment” was calculated by measuring the height of sediment (mm) and total volume (mm) in the glass bottle using calipers. The results are presented in Table 18 (pin-milled) and Table 19 (jet-milled).

TABLE 18
Visual Appearance Over Time (Pin & Jet-milled) At Ambient Temperature

| Sample | After 24 hours | After 7 days | Ease of Redispersion |
|---|---|---|---|
| 11A | Yellow suspension, | Yellow suspension, | Gentle agitation. |
| | 15.7% sediment | 14.7% sediment | ~30 inversions over |
| | | | 15 seconds. |
| 12A | Yellow suspension, | Yellow suspension, | Gentle agitation. |
| | 23.9% sediment | 21.5% sediment | ~15 inversions over |
| | | | 10 seconds. |
| 13A | Yellow suspension, | Yellow suspension, | Gentle agitation. |
| | 28.5% sediment | 25.2% sediment | ~15 inversions over |
| | | | 10 seconds. |
| 14A | Yellow suspension, | Yellow suspension, | Redisperses in ~3 |
| | 34.8% sediment | 30.5% sediment | inversions. |
| 15A | Yellow suspension, | Yellow suspension, | Redisperses in ~3 |
| | 33.9% sediment | 34.7% sediment | inversions. |
| 16A | Yellow suspension, | Yellow suspension, | Gentle agitation. |
| | slightly thickened | gel-like and almost | ~15 inversions over |
| | No noticeable | solid. | 10 seconds. |
| | sediment | | |
| 11B | Yellow suspension, | Yellow suspension, | Gentle agitation. |
| | No noticeable | 14.1% sediment | ~15 inversions over |
| | sediment | | 10 seconds. |
| 12B | Yellow suspension, | Yellow suspension, | Gentle agitation. |
| | No noticeable | 12.9% sediment | ~15 inversions over |
| | sediment | | 10 seconds. |
| 13B | Yellow suspension, | Yellow suspension, | Gentle agitation. |
| | No noticeable | 14.1% sediment | ~15 inversions over |
| | sediment | | 10 seconds. |
| 14B | Yellow suspension, | Yellow suspension, | Redisperses in ~3 |
| | No noticeable | 22.4% sediment | inversions. |
| | sediment | | |
| 15B | Yellow suspension, | Yellow suspension, | Redisperses in ~3 |
| | No noticeable | 23.6% sediment | inversions. |
| | sediment | | |
| 16B | Yellow suspension, | Yellow, gel-solid | Requires vigorous |
| | Highly viscous, | like structure. | agitation to redisperse. |
| | solid-like gel. | | |

TABLE 19
Visual Appearance Over 7 Days (Pin & Jet-milled) At 50° C.

| Sample | Observation after 7 days at 50° C. | Ease of Redispersion |
|---|---|---|
| 11A | Yellow suspension, solid flocculated | Gentle agitation. |
| | material adhered to bottle wall. | ~30 inversions over |
| | 31.5% sediment | 15 seconds. |
| 12A | Yellow suspension, solid flocculated | Gentle agitation. |
| | material adhered to bottle wall. | ~30 inversions over |
| | 29.9% sediment | 15 seconds. |
| 13A | Yellow suspension, solid flocculated | Gentle agitation. |
| | material adhered to bottle wall. | ~30 inversions over |
| | 33.0% sediment | 15 seconds. |
| 14A | Yellow suspension, solid flocculated | Gentle agitation. |
| | material adhered to bottle wall. | ~30 inversions over |
| | 42.1% sediment | 15 seconds. |
| 15A | Yellow suspension, solid flocculated | Gentle agitation. |
| | material adhered to bottle wall. | ~30 inversions over |
| | 42.0% sediment | 15 seconds. |
| 16A | Yellow suspension, gel-like and | Gentle agitation. |
| | almost solid. | ~30 inversions over |
| | | 15 seconds. |
| 11B | Yellow suspension, Large amount of | Gentle agitation. |
| | solid flocculated material adhered to | ~30 inversions over |
| | bottle wall. 22.2% sediment. | 15 seconds. |
| 12B | Yellow suspension, Large amount of | Gentle agitation. |
| | solid flocculated material adhered to | ~30 inversions over |
| | bottle wall. Difficult to quantify | 15 seconds. |
| | sediment. | |
| 13B | Yellow suspension, Large amount of | Gentle agitation. |
| | solid flocculated material adhered to | ~30 inversions over |
| | bottle wall. Difficult to quantify | 15 seconds. |
| | sediment. | |
| 14B | Yellow suspension, Large amount of | Gentle agitation. |
| | solid flocculated material adhered to | ~30 inversions over |
| | bottle wall. Difficult to quantify | 15 seconds. |
| | sediment. | |
| 15B | Yellow suspension, Large amount of | Gentle agitation. |
| | solid flocculated material adhered to | ~30 inversions over |
| | bottle wall. Difficult to quantify | 15 seconds. |
| | sediment. | |
| 16B | Yellow, gel-solid like structure. | Gentle agitation. |
| | | ~30 inversions over |
| | | 15 seconds. |

### (b) Repeat Sedimentation Testing and Assay Results Over Time

To confirm the trends and rates of sedimentation identified visually, a repeat sedimentation test was performed using the following six samples previously described in Tables 14A-14B (pin-milled) and Tables 15A-15B (jet-milled):
Samples 13A and 13 B (9.0 mg/mL povidon K30-pin-milled and jet-milled);
Samples 14A and 14B (6.0 mg/mL povidon K30 and 5.0 mg/mL AEROSIL®, pin-milled and jet-milled); and
Samples 15A and 15B (6.0 mg/mL povidon K30 and 5.0 mg/mL SYLOID® 244 FP, pin-milled and jet-milled).

The repeat testing was performed on the above samples that had been previously packed into 60 mL clear glass bottles (samples were ~14 days old). Samples were shaken vigorously to re-suspend any sediment 24 hours prior to the investigation starting.

On the day of analysis, each formulation was shaken vigorously for 20 seconds (vertically over a 30 cm distance twice a second) and left to settle for 5 minutes to dispel some of the foam and aeration of the formulation. After the sample had settled, 5.0 mL was taken by use of automated pipette with the tip 5-10 mm below the surface of the suspension and weighed in to a 200 mL flask. This was then filled to ~180 mL with diluent (50:50 MeCN:H₂O) and sonicated for 10 minutes. The sample was allowed to equilibrate to room temperature before making to volume.

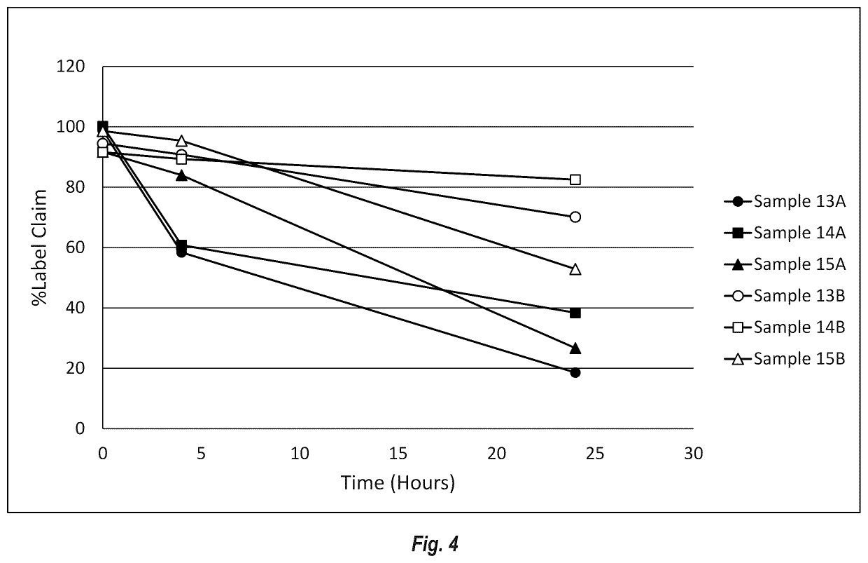

Samples were tested at T=0, 4 hours and 24 hours. The samples were not shaken at the T=4 hr or the T=24 hr time points to see how much of the belumosudil had settled out of the formulation. After the T=24 hr sample was taken, the remaining sample was shaken again and a sample taken to see if the belumosudil did re-suspend. The assay results not corrected for sample weight are shown in Table 20 and plotted in FIG. 4.

TABLE 20
Sedimentation Assay Results Over Time for Samples 13-15A/B

| | | | | | | | T = 24 After | |
|---|---|---|---|---|---|---|---|---|
| | T = 0 | | T = 4 | | T = 24 | | Shaking | |
| | | % | | % | | % | | % |
| | Assay | Label | Assay | Label | Assay | Label | Assay | Label |
| Sample | mg/mL | Claim | mg/mL | Claim | mg/mL | Claim | mg/mL | Claim |
| 13A | 39.25 | 98.1 | 23.44 | 58.6 | 7.47 | 18.7 | 45.06 | 112.7 |
| 14A | 40.05 | 100.1 | 24.41 | 61.0 | 15.38 | 38.4 | 43.26 | 108.2 |
| 15A | 36.63 | 91.6 | 33.54 | 83.9 | 10.72 | 26.8 | 45.81 | 114.5 |
| 13B | 38.01 | 95.0 | 36.14 | 90.4 | 27.96 | 69.9 | 41.21 | 103.0 |
| 14B | 36.69 | 91.7 | 35.55 | 88.9 | 32.78 | 81.9 | 41.36 | 103.4 |
| 15B | 39.27 | 98.2 | 37.96 | 94.9 | 21.13 | 52.8 | 46.43 | 116.1 |

Initial Sampling: The results at the initial sampling stage (T=0) showed results within the proposed specification limits of 90-110% of the label claim (or target concentration) of 40 mg/mL for all formulations. These results suggest that most, if not all, of the sediment in the samples was broken up and re-suspended.

Samples 15A and 14B had unexpectedly low assay values at the initial time-point. These results suggest that the belumosudil may have stuck to the walls of the bottle where the foam had dried and may not have been fully re-dispersed. Even though the initial values were low, they offered a baseline to indicate rates of sedimentation across the 24-hour testing period.

After initial shaking, the amount of foam produced by all samples was similar. However, after the initial 5 minutes, the jet-milled API samples had little to no foam, whereas the pin-milled samples had substantial foam that adhered to the pipette tip and had to be removed before the sample was transferred. Reduction in foaming with the jet-milled samples was favorable, despite earlier results indicating the jet-milled formulations may be problematic.

T=4 hours: From the assay results, the jet-milled API remained better suspended with assay values greater than 88%, compared to the assay values for the samples containing pin-milled API which ranged between 58-84%. The pin-milled sample containing only povidon K30 (13A), had the lowest assay value confirming that the addition of colloidal silica aids in the suspension of the belumosudil was beneficial. As among all samples, the assay value for the jet-milled sample containing SYLOID® was highest.

After standing for 4 hours, the foam in the pin-milled samples was still visible but was easier to sample from with most of the foam adhering on the walls of the bottle and not the pipette tip. At 4 hours, there was minimal foam in the jet-milled samples reflecting consistent reduction in foam with the jet-milled formulation at both T=0 and T-4 hrs.

T=24 hours: At the 24-hour timepoint, all formulation assays were below 85%, although samples manufactured using the jet-milled API still had higher assay results than samples using the pin-milled API. As with the T=4 hour samples, the formulations containing SYLOID® and AREOSIL® had higher assay values compared to the samples containing povidone K30 on its own, confirming that at 24 hours the addition of colloidal silica aided in suspending the belumosudil. At the 24-hour time point, the sample containing jet-milled API in the povidone only formulation provided a higher assay result compared to the formulation with SYLOID® suggesting a higher rate of sedimentation in the sample containing SYLOID®. Previous sedimentation testing, however, indicated this sediment was more loosely packed.

After 24 hours standing, the foam from the pin-milled samples had fully adhered to the walls of the bottle and the formulation had clear separation between the API sediment and the vehicle; with the jet-milled samples, the layers were not as clear.

T=24 hours (Following Shaking to Redisperse): The results after being shaken at the 24-hour time point show high assay values outside of the proposed specification limits of 90-110% of the target concentration. This was attributed to the assay values at the T=4 hours and T=24 hours' time points being low, meaning that there was more API in a smaller volume to be re-dispersed. Although these results are outside of the specification limits, it does suggest that the belumosudil was easily re-suspended in the vehicles with shaking.

The pH of the samples 11A to 16A and 11B to 16B were also recorded after 7 days at ambient temperature and at 50° C. No significant changes were observed in any of the samples after 7 days at ambient temperature (pH values ranged from 3.2 to 3.6 at both the initial testing and after 7 days). After 7 days at 50° C., a drop in pH was observed, i.e., pH ranged from 2.7 to 2.9 for all samples.

From this study, it was concluded that samples containing silicon dioxide performed best in terms of ease of resuspension after storage for 7 days. Although the assay results suggest AEROSIL® offered some benefits over SYLOID® 244 FP in reducing rates of sedimentation, samples containing SYLOID® 244FP were shown to cake less and were resuspended more easily. As all samples had visually low viscosities and high rates of sedimentation over time, ease of redispersion was considered an important characteristic, in particular when considering a product with a longer shelf life. SYLOID® 244FP also provided benefits over AEROSIL® 200 with ease of handling during manufacture. For these reasons, overall SYLOID® 244FP is considered a better option.

Determining the level of povidone requires a meticulous balance. Povidone K30 at higher levels were required to reduce sedimentation; however, the maximum level of inclusion for povidone K30 is impacted by the acceptable daily limit (ADI) for this compound of 50 mg/kg by weight; thus, levels need to be aligned with the dosing regime for administration to a subject.

The selection of pin-milled API versus jet-milled API also involved consideration of many intricate factors. As discussed in Examples 3, 4 and 5, various experiments instructed that pin-milled was preferred. Pin-milled was easier to handle than jet-milled API with a better flow observed. Pin-milled API also wetted more easily. When flocculation occurred, it was more severe in samples containing jet-milled API, and the jet-milled API was seen to adhere to the centrifuge wall.

Both the pin-milled and jet-milled samples redispersed easily, with no significant differences between the API. Lower amounts of sediment were observed following 7 days in batches containing jet-milled API.

Foaming occurred in samples containing pin-milled and jet-milled API once agitated. Samples containing jet-milled API appeared to foam slightly less.

Accordingly, there are many reasons leading one to conclude, initially, that pin-milled API would be a preferred option for pharmaceutical development. The applicants herein discovered, nonetheless, that jet-milled is a preferred method for pharmaceutical development in view of, inter alia, long-term stability considerations and lower amounts of sedimentation with jet-milled product that may be more noticeable only over longer periods of time on storage.

### (c) Further Povidone Comparative Testing

As shown above in Examples 6 (a) and (b), the formulations containing povidone K30 had relatively high rates of sedimentation. In this study, the longer chain PVP, povidone 90F, was investigated to determine whether it may provide a higher viscosity suspension to reduce rates of sedimentation. To this end, formulations having the compositions set forth in Table 21 were prepared.

TABLE 21
Composition of Povidone 90 Samples with Addition of Tropical Fruit Blend Flavor

| | Sample | | | | | | | |
|---|---|---|---|---|---|---|---|---|
| | 16-01 | | 16-02 | | 16-03 | | 16-04 | |
| | | g/100 mL | | g/100 mL | | g/100 mL | | g/100 mL |
| Component | mg/mL | Batch | mg/mL | Batch | mg/mL | Batch | mg/mL | Batch |
| Belumosudil | 48.496 | 4.8496 | 48.496 | 4.8496 | 48.496 | 4.8496 | 48.496 | 4.8496 |
| (Pin-milled) | | | | | | | | |
| Sodium | 0.50 | 0.05 | 0.50 | 0.05 | 0.50 | 0.05 | 0.50 | 0.05 |
| Benzoate | | | | | | | | |
| Povidone | 3.00 | 0.30 | 6.00 | 0.60 | 9.00 | 0.90 | 9.00 | 0.90 |
| 90 F | | | | | | | | |
| SYLOID ® | 5.00 | 0.50 | 5.00 | 0.50 | 5.00 | 0.50 | n/a | n/a |
| 244 FP | | | | | | | | |
| AEROSIL ® | n/a | n/a | n/a | n/a | n/a | n/a | 5.00 | 0.50 |
| 200 | | | | | | | | |
| Sucralose | 1.50 | 0.15 | 1.50 | 0.15 | 1.50 | 0.15 | 1.50 | 0.15 |
| Tropical | 4.00 | 0.40 | 4.00 | 0.40 | 4.00 | 0.40 | 4.00 | 0.40 |
| Fruit Blend | | | | | | | | |
| Flavour | | | | | | | | |
| Tartaric | to | to | to | to | to | to | to | to |
| Acid (q.s.) | pH 3.5 | pH 3.5 | pH 3.5 | pH 3.5 | pH 3.5 | pH 3.5 | pH 3.5 | pH 3.5 |
| Sterile Water | 1 | 100 | 1 | 100 | 1 | 100 | 1 | 100 |
| for Irrigation | | | | | | | | |
| (To vol./mL) | | | | | | | | |

The formulations of Table 21 were prepared using the following steps:
1. To a pre-calibrated beaker, about 80 mL of sterile water for irrigation was added;
2. While stirring, sodium benzoate, sucralose and tropical fruit blend flavor were added and mixed until dissolved;
3. While mixing, povidone 90F was added and mixed until a uniform mixture was observed;
4. If required, while mixing AEROSIL® 200 or SYLOID® 244FP were added and mixed until uniform;
5. While mixing, belumosudil was added and mixed until a uniform mixture was observed;
6. The pH was measured, and if required, tartaric acid was added in aliquots until pH 3.5±0.2 was achieved; and
7. Sterile water for irrigation was added to make up volume and the final pH was recorded.

Samples 16-01 to 16-04 were tested for sedimentation. 2 mL of each sample was added to a 15 mL plastic centrifuge tube, and the sedimentation was monitored over 7 days at room temperature. Sedimentation results at initial, after 2 hours and after 24 hours were recorded.

The results from this sedimentation testing are outlined in Table 22. Previous results for samples 14A and 15A (Table 16, above) are included for comparison.

TABLE 22
Sedimentation Results for Formulations Containing Povidone 90

| | Sample | | | | | |
|---|---|---|---|---|---|---|
| | 16-01 | 16-02 | 16-03 | 16-04 | 15A* | 14A* |
| | Details | | | | | |
| | 3 mg/mL | 6 mg/mL | 9 mg/mL | 3 mg/mL | 6 mg/mL | 6 mg/mL |
| | Povidone | Povidone | Povidone | Povidone | Povidone | Povidone |
| | 90F + | 90F + | 90F + | 90F + | K30 + | K30 + |
| | 5 mg/mL | 5 mg/mL | 5 mg/mL | 5 mg/mL | 5 mg/mL | 5 mg/mL |
| | Syloid 244FP | Syloid 244FP | Syloid 244FP | Aerosil 200 | Syloid 244FP | Aerosil 200 |
| Time | Level of Sediment | | | | | |
| Initial | None | None | None | None | None | None |
| 2 hs | 9.25 mL | 10.25 mL | 11.75 mL | 11.75 mL | <0.1 mL | <0.1 mL |
| | loosely | loosely | loosely | loosely | hard | hard |
| | packed | packed | packed | packed | sediment. | sediment. |
| | sediment, | sediment, | sediment, | sediment, | 4.5 mL | 4.5 mL |
| | 2.75 mL | 1.75 mL | 0.25 mL | 0.25 mL | loosely | loosely |
| | supernatant | supernatant | supernatant | supernatant | packed | packed |
| | | | | | sediment, | sediment, |
| | | | | | 7.5 mL | 7.5 mL |
| | | | | | supernatant | supernatant |
| 24 h | 7.5 mL | 8 mL | 8.5 mL | 10 mL | <0.1 mL | <0.1 mL |
| | loosely | loosely | loosely | loosely | hard | hard |
| | packed | packed | packed | packed | sediment. | sediment. |
| | sediment, | sediment, | sediment, | sediment, | 3.5 mL | 3.5 mL |
| | 4.5 mL | 4 mL | 3.5 mL | 2 mL | loosely | loosely |
| | supernatant | supernatant | supernatant | supernatant | packed | packed |
| | | | | | sediment, | sediment, |
| | | | | | 8.5 mL | 8.5 mL |
| | | | | | supernatant | supernatant |
| 7 days | 7 mL | 7.25 mL | 7.5 mL | 9 mL | <0.1 mL | <0.1 mL |
| | loosely | loosely | loosely | loosely | hard | hard |
| | packed | packed | packed | packed | sediment. | sediment. |
| | sediment, | sediment, | sediment, | sediment, | 3.5 mL | 3.5 mL |
| | 5 mL | 4.75 mL | 4.5 mL | 3 mL | loosely | loosely |
| | supernatant | supernatant | supernatant | supernatant | packed | packed |
| | | | | | sediment, | sediment, |
| | | | | | 8.5 mL | 8.5 mL |
| | | | | | supernatant | supernatant |
| Inversions | Fully | Fully | Fully | Fully | Fully | Fully |
| to | dispersed | dispersed | dispersed | dispersed | dispersed | dispersed |
| reconstitute | in 10 | in 10 | in 6 | in 10 | in 7 | in 15 |
| | inversions | inversions | inversions | inversions | inversions | inversions |

*Results previously obtained and reported in Table 16; included here for comparison.

The sedimentation testing demonstrated that the use of povidone 90F reduced the level of sedimentation when compared to the povidon K30 samples (samples 14A and 15A), with the majority remaining suspended after up to 7 day at ambient temperature.

Sample 16-03 containing SYLOID® 244 FP required fewer inversions to redisperse sediment after 7 days at ambient temperature when compared to sample 16-04 containing AEROSIL®200 which could be more beneficial over longer term storage. As such, SYLOID® 244FP was considered the preferred silica for inclusion in the belumosudil formulation.

Samples 16-01 to 16-04 were also further evaluated over time with accelerated storage conditions. 30 mL of each sample was transferred to a 2×60 mL glass bottle and stored at ambient temperature and at 40° C./75% RH, to be tested after 7 days for visual appearance, level of sedimentation, redispersion of any sediment and pH. With this study, it was found that when stored at ambient temperature for T=7 days, all 4 samples stored in 60 mL clear glass bottles, containing povidon 90F, were easily reconstituted with gentle shaking for ~10 seconds. Foaming was observed following redispersion after storage for 7 days at ambient temperature, even with the samples containing flavor.

For samples at T=7 at 40° C./75% RH, all 4 samples containing povidone 90F appeared more viscous after storage at 40° C./75% RH but were redispersed with gentle shaking for about 10 seconds. No foaming was observed.

Lastly, four samples were selected to be studied for XRPD analysis, specifically, samples 15A and 16A (Table 14 B), sample 17 (Table 23), and sample 16-03 (Table 21). 30 mL of each sample was transferred to 1×60 mL glass bottle for analytical testing at initial and after seven days at ambient temperature and accelerated storage conditions (i.e., 50° C.) (with samples agitated to redisperse any sediment). Samples were isolated for XRPD analysis. The XRPD data demonstrated that all samples were consistent with the original form of the API and pH drift is not associated with a change in form of API. The levels of sodium benzoate included at 0.5 mg/mL did not present a concern.

### Example 7: Preservatives and Sodium Benzoate Levels

Sodium benzoate was selected as the preferred choice of preservative based on the low natural pH of API in water of ~pH 3 and the instability observed in samples containing potassium sorbate which is described in Example 1.

Four formulations were prepared to assess the impact of adjusting the sodium benzoate levels and select a preferred level to use in a belumosudil formulation. Specifically, the four samples 17 to 20 described in Table 23 were prepared which included high, mid and low levels of sodium benzoate in combination with povidon K30 at 9 mg/mL and SYLOID® 244FP at 5 mg/mL, the latter having been identified as preferred suspending agents based on findings in prior Examples 3 (povidon) and 6 (SYLOID® 244FP). Sample 20 containing no preservative was manufactured for reference.

TABLE 23
Formulations for Assessing Sodium Benzoate Levels

| | Sample | | | | | | | |
|---|---|---|---|---|---|---|---|---|
| | 17 | | 18 | | 19 | | 20 | |
| | Description | | | | | | | |
| | High level sodium | | Mid level sodium | | Low level sodium | | No sodium | |
| | benzoate | | benzoate | | benzoate | | benzoate | |
| | | g/150 mL | | g/150 mL | | g/150 mL | | g/150 mL |
| Components | mg/mL | Batch | mg/mL | Batch | mg/mL | Batch | mg/mL | Batch |
| Belumosudil | 48.496* | 7.27 | 48.496* | 7.27 | 48.496* | 7.27 | 48.496* | 7.27 |
| (pin-milled) | | | | | | | | |
| Sodium | 1.00 | 0.15 | 0.50 | 0.075 | 0.25 | 0.0375 | n/a | n/a |
| Benzoate | | | | | | | | |
| Povidone K30 | 9.00 | 1.35 | 9.00 | 1.35 | 9.00 | 1.35 | 9.00 | 1.35 |
| SYLOID | 5.00 | 0.75 | 5.00 | 0.75 | 5.00 | 0.75 | 5.00 | 0.75 |
| ®244 FP | | | | | | | | |
| Tartaric | to | to | to | to | to | to | to | to |
| Acid (Q.s.) | pH 3.5 | pH 3.5 | pH 3.5 | pH 3.5 | pH 3.5 | pH 3.5 | pH 3.5 | pH 3.5 |
| Sterile Water | 1 | 150 | 1 | 150 | 1 | 150 | 1 | 150 |
| (to volume/mL) | | | | | | | | |

*48.496 mg/mL equivalent to 40 mg/mL freebase adjusted with 1.2124 salt correction factor

The above formulations (samples 17-20), were prepared to the volume of 150 mL using the following manufacturing steps:
1. To a 250 mL glass beaker, 130 mL of sterile water for irrigation was added;
2. While stirring using a magnetic stirrer, sodium benzoate was added and mixed until fully dissolved;
3. While stirring, povidon K30 was added and mixed until fully dissolved;
4. While stirring, SYLOID®244 FP was added and mixed until dispersed and a uniform mixture obtained;
5. While stirring, belumosudil (pin-milled) was added and mixed until dispersed and a uniform mixture was obtained;
6. The pH was recorded and if required, tartaric acid was added to adjust to reach the target pH of 3.5±0.2;
7. Sterile water for irrigation was added to make up the volume and the formulaton mixed until uniform.

PET testing was performed on samples 17 to 20. The results demonstrated that all batches containing sodium benzoate at all levels (0.25 mg/mL, 0.50 mg/mL and 1.00 mg/mL) provided suitable preservative efficacy. The results confirm that all the levels to sodium benzoate may be considered as alternative options and an intermediate level of 0.50 mg/mL would be a suitable level of inclusion of sodium benzoate (to allow for any potential degradation of sodium benzoate on longer term stability.) Sample 20 containing no sodium benzoate failed to provide adequate protection from microbial growth. Thus, it is advantageous to include the preservative in a ready-to-use liquid formulation comprising belumosudil.

### Example 8: Impact of Sweeteners and Flavors

A series of vehicles including sweeteners and flavors were developed to identify those suitable for reconstitution of belumosudil in bottle and potential administration to subjects. Initial formulation development studies were conducted at a small-scale batch of up to 100 mL. First, vehicles were manufactured according to the composition details presented in Tables 24A-24B and Tables 25A-25B.

The levels of sweetener were selected based on the findings that the API has a bitter profile which requires improvement in palatability for the formulation to be effectively received by subjects. The vehicles were prepared by (a) adding the required quantity of sterile water for irrigation into a 125 mL clear glass bottle; (2) adding the required quantity of sweetener and mixing using a vortex mixer until visually dissolved; and (3) adding the required quantity of flavor (where applicable) and mixing using a vortex mixer until visually dissolved.

TABLE 24A
Composition Details of Vehicles Containing
Sucralose (Vehicle 1-3)

| | Vehicle 01 | Vehicle 02 | Vehicle 03 |
|---|---|---|---|
| Component | % w/w | % w/w | % w/w |
| Sucralose | 0.15 | 0.30 | 0.15 |
| Orange Flexarome Flavor | n/a | n/a | 0.40 |
| 880021 TFS504 | | | |
| Tropical Fruit Blend Flavor | n/a | n/a | n/a |
| PGS-145298 | | | |
| Lemon PS PHS-135460 | n/a | n/a | n/a |
| Orange Flavor | n/a | n/a | n/a |
| SC611927 | | | |
| Sterile Water for Irrigation | To 100 | To 100 | To 100 |
| (To volume/mL) | | | |

TABLE 24B
Composition Details of Vehicles Containing
Sucralose (Vehicles 4-6)

| | Vehicle 04 | Vehicle 05 | Vehicle 06 |
|---|---|---|---|
| Component | % w/w | % w/w | % w/w |
| Sucralose | 0.15 | 0.15 | 0.15 |
| Orange Flexarome Flavor | n/a | n/a | n/a |
| 880021 TFS504 | | | |
| Tropical Fruit Blend Flavor | 0.40 | n/a | n/a |
| PGS-145298 | | | |
| Lemon PS PHS-135460 | n/a | 0.40 | n/a |
| Orange Flavor | n/a | n/a | 0.40 |
| SC611927 | | | |
| Sterile Water for Irrigation | To 100 | To 100 | To 100 |
| (To volume/mL) | | | |

TABLE 25A
Composition Details of Vehicles Containing
Acesulfame K (Vehicles 7-9)

| | Vehicle 07 | Vehicle 08 | Vehicle 09 |
|---|---|---|---|
| Component | % w/w | % w/w | % w/w |
| Acesulfame K | 0.45 | 0.90 | 0.45 |
| Orange Flexarome Flavor | n/a | n/a | 0.40 |
| 880021 TFS504 | | | |
| Tropical Fruit Blend Flavor | n/a | n/a | n/a |
| PGS-145298 | | | |
| Lemon PS PHS-135460 | n/a | n/a | n/a |
| Orange Flavor | n/a | n/a | n/a |
| SC611927 | | | |
| Sterile Water for Irrigation | To 100 | To 100 | To 100 |

TABLE 25B
Composition Details of Vehicles Containing
Acesulfame K (Vehicles 10-12)

| | Vehicle 10 | Vehicle 11 | Vehicle 12 |
|---|---|---|---|
| Components | % w/w | % w/w | % w/w |
| Acesulfame K | 0.45 | 0.45 | 0.45 |
| Orange Flexarome Flavor | n/a | n/a | n/a |
| 880021 TFS504 | | | |
| Tropical Fruit Blend Flavor | 0.40 | n/a | n/a |
| PGS-145298 | | | |
| Lemon PS PHS-135460 | n/a | 0.40 | n/a |
| Orange Flavor SC611927 | n/a | n/a | 0.40 |
| Sterile Water for Irrigation | To 100 | To 100 | To 100 |

Vehicles 01-12 were each characterized for visual appearance and pH. Vehicles 01-02 (containing sucralose) and vehicles 07-08 (containing acesulfame k) were visually observed as clear, colorless solutions. The visual appearance of vehicles containing sweetener and flavor combinations were different with all batches observed as a cloudy liquid except for vehicles containing orange flavor (Vehicles 06 and 12) that were an off-white, opaque liquid. The pH of the vehicles ranged from a low of 4.3 (Vehicles 03 and 09), to a high of 7.0 (vehicles 01 and 02), which fell within proposed acceptance criteria.

Samples of reconstituted belumosudil were then prepared to assess the performance of Vehicles 01-12. For preparation of the reconstituted belumosudil in bottle, 727.44 mg of belumosudil mesylate salt (600 mg belumosudil free base) was transferred into 60 mL Type III clear glass bottles. Into the bottle, 15 mL of each of the vehicles as listed in Tables 24A-24B and 25A-25B were added and the samples were agitated to reconstitute.

All the samples were reconstituted with gentle agitation for 10 seconds. All the samples containing acesulfame K (using Vehicles 07-12), showed signs of flocculation after about 5 minutes and thickened significantly. As such, all samples containing acesulfame K were considered unsuitable for use in the belumosudil formulation. The visual appearance of samples containing sucralose (using Vehicles 01-06), were opaque yellow dispersions and showed no signs of flocculation. These vehicles containing sucralose were therefore considered suitable for use in the formulation. Additionally, the pH of the reconstituted samples using Vehicles 01-06 fell in the range of 3.1 to 3.2, a suitable pH for the final formulation without the need for pH adjustment.

Work was further carried out to investigate the effect the addition of flavor to the belumosudil formulation has on foaming following agitation. Tropical Fruit Blend Flavor was selected for this assessment and the experiment was performed using previously prepared sample 11 A (Table 14A), and sample 12B (Table 15A) (in above Example 6). Each sample included sodium benzoate as preservative and povidone K30 without addition of silica/suspending agents, wherein sample 11A included pin-milled belumosudil and sample 12B included the jet-milled drug.

For each sample, about 30 mL was transferred to a 60 mL glass bottle. To assess the foaming and effect on addition of flavor, the following experiment was carried out.
1. Each sample was agitated by shaking for 30 seconds (~2 inversion each second) and the visual appearance and foaming was recorded;
2. Each sample was left undisturbed until no foam/aeration was present;
3. 0.12 g of Tropical Fruit Blend Flavor (~0.4% w/v) was added to each sample which was mixed using a vortex mixer to dissolve.

Table 26 outlines the composition of the liquid formulation comprising belumosudil examined in this study after addition of flavor.

TABLE 26
Composition of Samples 11A and 12 B After Addition of Flavor

| | Sample | | | |
|---|---|---|---|---|
| | 11A- with Flavor | | 12B - With Flavor | |
| | | g/30 mL | | g/30 mL |
| Components | mg/mL | Batch | mg/mL | Batch |
| Belumosudil | 48.496 | 4.8496 | n/a | n/a |
| (Pin-Milled) | | | | |
| Belumosudil | n/a | n/a | 48.496 | 4.8496 |
| (Jet-Milled) | | | | |
| Sodium Benzoate | 0.50 | 0.05 | 0.50 | 0.05 |
| Povidone K30 | 3.00 | 0.30 | 6.00 | 0.60 |
| Tartaric Acid | Q.s. to | Q.s. to | Q.s. to | Q.s. to |
| | pH 3.5 | pH 3.5 | pH 3.5 | pH 3.5 |
| Sterile Water for | 1 | 30 | 1 | 100 |
| Irrigation (to | | | | |
| volume/mL) | | | | |
| Tropical Fruit | 4.00 | 0.12 | 4.00 | 0.12 |
| Flavour | | | | |

The following observations were noted.

With no flavor, a large amount of foam and aeration could be seen after agitation for 30 seconds. There was a noticeable difference in foaming with pin-milled and jet-milled API when no flavor was added, after standing undisturbed for 5 minutes. Sample 11A (pin-milled API) had more persistent foam compared to sample 12B (jet-milled API).

After the addition of flavor, only a very small amount of aeration was seen following agitation for 30 seconds. Once Tropical Fruit Blend flavor was added, there were no noticeable differences in the amount of foam or aeration in the samples containing pin-milled compared to jet-milled API. Accordingly, it was concluded that the tropical fruit blend flavor was suitable for use in the belumosudil formulation and anticipated that other alternative flavors listed in Tables 24A and 24B would have a similar effect on foaming based on prior observations with these flavors.

### Example 9: Comparative Analysis and Homogenization Step on Scale Up

A comparative analysis was performed on scaled-up systems containing 9 mg/mL povidone 90F and an alternative system containing 6 mg/mL povidone K30 with pin-milled API, along with the system containing 9 mg/mL povidone 90F using jet-milled API to allow comparison to the pin-milled API. In all formulations, SYLOID® 244FP was used at 5 mg/mL as a suspending agent, sodium benzoate was included at 0.5 mg/mL to offer preservative efficacy and lemon flavor was used.

Details of the compositions used in this study are further shown in Table 27.

TABLE 27
Composition Details for Comparative and Stability Studies

| | Sample | | | | | |
|---|---|---|---|---|---|---|
| | 21 | | 22 | | 23 | |
| Components | mg/mL* | g/2500 g | mg/mL* | g/2500 g | mg/mL* | g/2500 g |
| Belumosudil | 48.496* | 118.875 | 48.496* | 118.875 | n/a | n/a |
| (Pin-milled) | | | | | | |
| Belumosudil | n/a | n/a | n/a | n/a | 48.496* | 118.875 |
| (Jet-milled) | | | | | | |
| Sodium Benzoate | 0.500 | 1.225 | 0.500 | 1.225 | 0.500 | 1.225 |
| Povidone K30 | 6.00 | 14.700 | n/a | n/a | n/a | n/a |
| Povidone 90 F | n/a | n/a | 9.00 | 22.050 | 9.00 | 22.050 |
| Syloid 244 FP | 5.00 | 12.250 | 5.00 | 12.250 | 5.00 | 12.250 |
| Sucralose | 1.500 | 3.675 | 1.500 | 3.675 | 1.500 | 3.675 |
| Lemon Flavor | 4.000 | 9.800 | 4.000 | 9.800 | 4.000 | 9.800 |
| PHS-135460 | | | | | | |
| Tartaric | Q.s. to | Q.s. to | Q.s. to | Q.s. to | Q.s. to | Q.s. to |
| Acid | pH 3.5 | pH 3.5 | pH 3.5 | pH 3.5 | pH 3.5 | pH 3.5 |
| Sterile Water | To | To | To | To | To | To |
| for Irrigation | 1 mL | 2500 g | 1 mL | 2500 g | 1 mL | 2500 g |

Samples 21-23 were analyzed as to their bulk homogeneity as follows. Formulations were gently agitated by inversion 10 times prior to sampling to ensure any sediment was resuspended prior to testing.

Two replicate samples were taken from each bottle to provide n=6. 1.00 mL of suspension in to a 200 mL volumetric flask and made to about 180 mL with diluent (50:50 MeCN:H₂O) and sonicated for 10 minutes. Samples were allowed to equilibrate to room temperature before making to volume with diluent. Samples were then filtered through 0.45 μm PTFE filters discarding the first 2 mL to waste before vialling for analysis.

Assay values for both sodium benzoate and belumosudil from the initial assessment of bulk homogeneity were collected. The results are shown in Table 28.

TABLE 28
Bulk Homogeneity Data on Scale up with Samples 21-23

| | | Sodium Benzoate | | | Belumosudil | | |
|---|---|---|---|---|---|---|---|
| | Position | Assay | % Label | | Assay | % Label | |
| Sample | in Fill | (mg/mL) | Claim | % RSD | (mg/mL) | Claim | % RSD |
| 21 | Start | 0.519 | 103.7% | 3.88 | 52.143 | 130.36% | 3.89 |
| | | 0.505 | 101.0% | | 51.660 | 129.15% | |
| | Middle | 0.529 | 105.9% | | 50.108 | 125.27% | |
| | | 0.565 | 113.0% | | 55.181 | 137.95% | |
| | End | 0.517 | 103.5% | | 49.941 | 124.85% | |
| | | 0.528 | 105.7% | | 53.591 | 133.98% | |
| 22 | Start | 0.509 | 101.7% | 2.62 | 46.459 | 116.15% | 38.82 |
| | | 0.489 | 97.8% | | 47.251 | 118.13% | |
| | Middle | 0.481 | 96.3% | | 84.259 | 210.65% | |
| | | 0.518 | 103.6% | | 86.023 | 215.06% | |
| | End | 0.499 | 99.9% | | 38.683 | 96.71% | |
| | | 0.501 | 100.1% | | 39.153 | 97.88% | |
| 23 | Start | 0.523 | 104.6% | 3.45 | 32.020 | 80.05% | 46.83 |
| | | 0.491 | 98.3% | | 42.039 | 105.10% | |
| | Middle | 0.487 | 97.4% | | 37.177 | 92.94% | |
| | | 0.518 | 103.6% | | 37.907 | 94.77% | |
| | End | 0.484 | 96.8% | | 87.665 | 219.16% | |
| | | 0.488 | 97.6% | | 82.389 | 205.97% | |

The bulk homogeneity data demonstrated that the sodium benzoate assay content complied with the specification limit of RSD≤% 6 for all batches. However, the belumosudil assay content was significantly higher than the acceptance criteria of 90-100% for sample 21 and showed significant variation in samples 22 and 23, with levels that did not meet the specification limit of RSD≤% 6.

Viscosity testing was also carried out on samples 21-23 using a Brookfield DV1 LV cone and plater viscometer, using spindle CP41 at 30 RPM at 20° C. Results are shown in Table 29.

TABLE 29
Viscosity Testing Results for Comparative
Samples 21-23 on Scale up

| Sample | 21 | 22 | 23 |
|---|---|---|---|
| RPM | 100 | 100 | 100 |
| Viscosity (cps) | 6.02 | 4.82 | 5.53 |
| Torque (%) | 52.3 | 41.9 | 48.0 |
| Temperature (° C.) | 20 | 20 | 20 |

The viscosity values recorded were unexpectedly very low, in particular, for sample 22 containing povidon 90F. Previously systems had been observed to thicken over time, but this was not observed in samples 21-23.

While batches have been successfully manufactured on a smaller scale using only overhead mixing, it was determined from this study that on scale up, overhead mixing was insufficient to adequately disperse the API and form a homogeneous suspension, which contributed to the highly varied assay results in this Example. It was determined that a homogenization step is advantageously included on scale up to achieve a homogenous suspension. Further investigation confirmed the use of the Silverson homogenizer improved API homogeneity.

### Example 10: System Refinement and Advantageous Parameters

Following the scaleup work in Example 9, additional comparative samples were prepared and analyzed with the initial hypothesis of increasing the viscosity of the system to then reduce the rates of sedimentation in the PVP/formulation system. Two approaches were investigated to optimize the system:
1. Increasing levels of povidon 90F: PVP has been shown to be effective in wetting the API and can be used as a thickener. However, povidone 90F is to be increased to levels no higher than the acceptable ADI based on the mean body-weight of a female child of 3 months (5.5 kg) as per the Pharmaceutical Development and Control Strategy Plan. PVP has an ADI of 50 mg/kg body weight and so must not exceed 275 mg/dose (based on 1 dose/day)
2. Adding methocel K4M in combination with povidone K30: Previously, samples containing alternative suspending agents to PVP were observed to gel considerably over time and aggregate considerably. As PVP aids in the wetting of the API, work was carried out to investigate the use of an alternative suspending agent in combination with povidone K30 (based on available information that the shorter chain PVP was more likely to aid in wetting compared to povidon 90F). Methocel K4M was selected as the alternative thickener as it showed the least severe physical incompatibilities with belumosudil compared to the other suspending agents tested.

To investigate these approaches, fours formulations were prepared on a 200 g scale having the composition details outlined in Tables 30A and 30B. Samples were placed on a short-term stability study and assessed at T=14 days and T=1 month at 25° C./60% RH and at 40° C./75% RH for visual appearance, assay, pH and viscosity.

TABLE 30A
Formulations Use in Scale-Up Optimization Study (Samples 24-01 & 24-02)

| | Sample | | | | | |
|---|---|---|---|---|---|---|
| | 24-01 | | | 24-02 | | |
| | | | g/200 g | | | g/200 g |
| Components | mg/mL | % w/w | batch | mg/mL | % w/w | batch |
| Belumosudil | 48.496 | 4.75 | 9.51 | 48.496 | 4.75 | 9.51 |
| (Pin-milled) | | | | | | |
| Belumosudil | n/a | n/a | n/a | n/a | n/a | n/a |
| (Jet-milled) | | | | | | |
| Sodium Benzoate | 0.50 | 0.05 | 0.10 | 0.50 | 0.05 | 0.10 |
| Kollidon 90F | 30.00 | 2.94 | 5.88 | 50.00 | 4.90 | 9.80 |
| Kollidon K30 | n/a | n/a | n/a | n/a | n/a | n/a |
| Methocel K4M | n/a | n/a | n/a | n/a | n/a | n/a |
| SYLOID ® 244 FP | 5.00 | 0.49 | 0.98 | 5.00 | 0.49 | 0.98 |
| Sucralose | 1.500 | 0.147 | 0.294 | 1.500 | 0.147 | 0.294 |
| Lemon Flavor | 4.00 | 0.39 | 0.78 | 4.00 | 0.39 | 0.78 |
| Tartaric Acid | Q.s. to | Q.s. to | Q.s. to | Q.s. to | Q.s. to | Q.s. to |
| | pH 3.5 | pH 3.5 | pH 3.5 | pH 3.5 | pH 3.5 | pH 3.5 |
| Sterile Water | 1 | 100 | 200 | 1 | 100 | 200 |
| for Irrigation | | | | | | |
| Total | 1020** | 100 | 200 | 1020* | 100 | 200 |

| | | |
|---|---|---|
| Comment | Increased povidon | Increased povidon |
| | 90F to 30 mg/mL | 90F to 50 mg/mL. |
| | Equivalent to 150 mg | Equivalent to 250 mg |
| | PVP/5 mL dose. | PVP/5 mL dose |

TABLE 30B
Formulations Use in Scale-Up Optimization Study (Samples 24-03 & 24-04)

| | Batch | | | | | |
|---|---|---|---|---|---|---|
| | 24-03 | | | 24-04 | | |
| | | | g/200 g | | | g/200 g |
| Composition | mg/mL | % w/w | batch | mg/mL | % w/w | batch |
| Belumosudil | 48.496 | 4.75 | 9.51 | n/a | n/a | n/a |
| (Pin-milled) | | | | | | |
| Belumosudil | n/a | n/a | n/a | 48.496 | 4.75 | 9.51 |
| (Jet-milled) | | | | | | |
| Sodium Benzoate | 0.50 | 0.05 | 0.10 | 0.50 | 0.05 | 0.10 |
| Kollidon 90F | n/a | n/a | n/a | 30.00 | 2.94 | 5.88 |
| Kollidon K30 | 10.00 | 0.98 | 1.96 | n/a | n/a | n/a |
| Methocel K4M | 5.00 | 0.49 | 0.98 | n/a | n/a | n/a |
| SYLOID ® 244 FP | 5.00 | 0.49 | 0.98 | 5.00 | 0.49 | 0.98 |
| Sucralose | 1.500 | 0.147 | 0.294 | 1.500 | 0.147 | 0.294 |
| Lemon Flavor | 4.00 | 0.39 | 0.78 | 4.00 | 0.39 | 0.78 |
| Tartaric Acid | Q.s. to | Q.s. to | Q.s. to | Q.s. to | Q.s. to | Q.s. to |
| | pH 3.5 | pH 3.5 | pH 3.5 | pH 3.5 | pH 3.5 | pH 3.5 |
| Sterile Water | 1 | 100 | 200 | 1 | 100 | 200 |
| for Irrigation | | | | | | |
| Total | 1020** | 100 | 200 | 1020** | 100 | 200 |

| | | |
|---|---|---|
| Comment | 10 mg/mL Kollidon K30 | Increased povidone 90F to |
| | for API wetting | 30 mg/mL Equivalent to |
| | 5 mg/mL Methocel | 150 mg PVP/5 mL dose. |
| | K4M as thickener | Prepared using jet-milled API. |

All four systems (24-01 to 24-04), appeared more viscous than seen with previous formulations. At initial mixing, all four systems were flowable and the API wetted well. On standing for 24 hours, sample 24-03 containing methocel K4M became semi-solid but changed to a fluid state following 10 seconds of shaking. After one week, samples 24-01, 24-02 and 24-04 all had some sedimentation. Sample 24-01 appeared to have the highest amount of sediment, when compared to samples 24-02 (containing higher levels of povidone 90F) and sample 24-04 (containing the same level of povidone 90F but prepared using jet-milled API.) Sample 24-03 showed no signs of sedimentation but was semi-solid.

At T=14 days and T=28 days, the samples were re-assessed for visual appearance. Sample 24-03 remained highly viscous, opaque yellow and gel-like but was easily liquified with shaking to form an opaque yellow liquid. Systems 24-01, 24-02 and 24-04 were flowable opaque yellow liquids with a small amount of sedimentation that was redispersed with shaking.

It was noted that “banding” was seen where API adhered to the wall of the glass bottles. Interestingly and surprisingly, more API was seen adhering to the walls with systems containing pin-milled API compared to jet-milled API (i.e., samples 24-03 and 24-04). Accordingly, the following conclusions were made:
1. System 24-03 containing Methocel K4M formed a solid/gel-like structure. However, this was easily shaken to form a liquid again. This remained liquid like but regained its structure after 24 hours.
2. Of the remaining samples, 24-04 with 30 mg/mL povidone 90F and using jet-milled API had the lowest amount of sediment. Sample 24-01, containing 30 mg/mL povidone 90F and using pin-milled API, had the highest rate of sedimentation and was the most likely to adhere to the glass bottle.
3. Sample 24-02 containing 50 mg/mL povidone 90F manufactured using pin-milled API had a reduced rate of sedimentation compared to sample 24-01.

From this study, it was determined that a system comprising povidone 90F using jet-milled belumosudil (sample 24-04), unexpectedly provided the best results on visual examination with the lowest amount of sedimentation.

All four samples were also analyzed via sodium benzoate and belumosudil assays. For all samples, the assay data for both the belumosudil and sodium benzoate was comparable to each other. Samples obtained with a Push in Bottle Adaptor (PIBA) provided comparable results to samples taken using an automated pipette showing that either method of sample transfer was suitable.

Samples 24-02 to 24-04 for both storage conditions and 24-01 for 25° C./60% RH had assay values for both the belumosudil and sodium benzoate that were within the specification limits of 90-110% of the label claim. For samples 24-02 to 24-04, there was no significant change in the assay data when compared to the initial and 14-day timepoint.

The pH values for the four samples at initial, T=2 weeks, and T=1 month at 25° C./60% RH and 40° C./75% RH are outlined in Table 31.

TABLE 31
pH Results for System Optimization (Samples 24-01 to 24-04)

Sample

| | | | |
|---|---|---|---|
| 24-01 | 24-02 | 24-03 | 24-04 |

| | |
|---|---|
| Time point | pH |

| | | | | |
|---|---|---|---|---|
| Initial | 3.6 | 3.6 | 3.7 | 3.4 |
| T = 2 weeks, | 3.2 | 3.3 | 3.4 | 3.2 |
| 25° C./60% RH | | | | |
| T = 2 weeks, | 3.0 | 2.9 | 3.1 | 2.8 |
| 40° C./75% RH | | | | |
| T = 4 weeks, | 3.1 | 3.1 | 3.4 | 3.0 |
| 25° C./60% RH | | | | |
| T = 4 weeks, | 2.9 | 2.8 | 3.1 | 2.7 |
| 40° C./75% RH | | | | |

A pH drift downward was seen in all systems, with the largest change observed at 40° C./75% RH. Initial pH preservative stability work indicated that in samples where pH drift is seen, the pH tended to drop to about pH 2.9. Benzoic acid is most effective at pH 3 so even with the downward drift this pH would be acceptable for preservative efficacy. The pH preservative stability study suggested there was no chemical changes that occurred at this pH. XRPD analysis suggests samples at pH 3 had no form change unless in the presence of a citrate buffer as addressed in Example 2.

Due to observations of the tendency of the pH to drift towards ~pH 3.0 over time then remain at this approximate pH value, and given the stability of the belumosudil at this value, pH 3.0±0.5 was targeted as a suitable pH range for the liquid formulation comprising belumosudil.

The viscosity of the four systems was also tested using a Brookfield cone and plate viscometer, spindle CP52, tested at 20° C. Samples were shaken vigorously for 10 seconds prior to testing to disperse any sediment. Table 32 outlines viscosity data at Initial, T=2 weeks and T=4 weeks at 25° C./60% RH and 40° C./75% RH.

TABLE 32
Viscosity Data for Samples 24-01 to 24-04 at Initial,
T = 14 day and T = 28 day

Sample

| | | | |
|---|---|---|---|
| 24-01 | 24-02 | 24-03 | 24-04 |

| | |
|---|---|
| Time point | Viscosity (cp) at 30 rpm (20° C.) |

| | | | | |
|---|---|---|---|---|
| Initial | 31.0 | 64.5 | 192.3 | 40.0 |
| T = 2 weeks, | 45.3 | 79.4 | 221.7 | 36.1 |
| 25° C./60% RH | | | | |
| T = 2 weeks, | 45.6 | 64.8 | 258.0 | 32.0 |
| 40° C. /75% RH | | | | |
| T = 4 weeks, | 57.4 | 59.2 | 268.9 | 39.5 |
| 25° C./60% RH | | | | |
| T = 4 weeks, | 53.3 | 83.1 | 256.8 | 27.5 |
| 40° C. /75% RH | | | | |

As can be seen, slight variations in viscosity were observed, but there was no significant change from initial and after 2 weeks and 4 weeks at 25° C./60% RH and 40° C./75% RH. The trend between systems remained the same at all timepoints with the most viscous being sample 24-03 containing 10 mg/mL Povidone K30±5 mg/mL Methocel K4M (with jet-milled API), and the least viscous being sample 24-01 containing 30 mg/mL Povidone 90F. Surprisingly, the higher viscosity of sample 24-03 did not correlate to reduced sedimentation rates, as initially proposed. As indicated above, of all samples, 24-04 with 30 mg/mL povidone 90F and using jet-milled API had the lowest amount of sediment but also one of the lower viscosity rates.

Overall, upon comparative analysis, sample 24-02 (50 mg/mL povidone 90F) provided the most consistent assay results, in particular compared to sample 24-01 (30 mg/mL povidone 90F), which also used pin-milled API. Sample 24-03 showed thickening/gelling as was seen in the past but was more easily liquified when shaken and did not show the same agglomerates following shaking as previously seen in batches containing methocel K4M but without povidone K30.

However, it also was observed that the use of jet-milled API demonstrated improvement in the reduction of API “banding” on the glass bottle and reduction in rate of sedimentation compared to pin-milled. This had been observed comparing between samples 24-01 (pin-milled) and 24-04 (jet-milled) which both contain 30 mg/mL Povidone 90F. With preparation of up-scaled, development samples and increased levels of PVP, the agglomeration was not observed that had been noticed in early development when using jet-milled API. As such, from this study it was concluded that jet-milled belumosudil would be most advantageous for use in the liquid formulation comprising belumosudil. Reference is made to Table 33 in summarizing the components of the liquid formulation.

TABLE 33
Formulation Components and Analysis

| Component | mg/mL | % w/w | Comment |
|---|---|---|---|
| Belumosudil | 48.496 | 4.755 | Equivalent to 40 mg/mL adjusted using factor of 1.2124. Jet- |
| (jet-milled) | | | milled API preferred over pin-milled API based on lower |
| | | | rates of sedimentation seen when using jet-milled API and |
| | | | improvements seen on reducing API sticking to container |
| | | | wall. |
| Sodium | 0.500 | 0.049 | Sodium Benzoate is important for preservation based on PET |
| Benzoate | | | results for a sample without preservative. |
| | | | Sodium benzoate has both bacteriostatic and antifungal |
| | | | properties attributed to undissociated benzoic acid; hence, |
| | | | preservative efficacy is best seen in solutions between pH |
| | | | 2-5. The JECFA (1996) has set an ADI of up to 5 mg/kg body- |
| | | | weight. |
| Sucralose | 1.500 | 0.147 | Sucralose may be included at 1.5 mg/mL based on palatability |
| | | | during the taste assessment. |
| | | | An ADI of up to 15 mg/kg body weight was set in 2000. In |
| | | | 2016, EFSA concluded that the proposed extension for use of |
| | | | sucralose in foods for special medical purposes for young |
| | | | children aged from 1 to 3 years would not be of safety |
| | | | concern. |
| SYLOID ® | 5.000 | 0.490 | Experiments indicated that SYLOID ®244FP included at 5 |
| 244FP | | | mg/mL had a beneficial effect on preventing caking and ease |
| (Colloidal | | | of reconstitution compared to samples without colloidal |
| silica) | | | silica. |
| | | | Silicon dioxide (E 551) is authorized as a food additive in the |
| | | | European Union (EU) in accordance with Annex II and |
| | | | Annex III to Regulation (EC) No 1333/2008 on food additives |
| | | | and specific purity criteria are defined in the Commission |
| | | | Regulation (EU) No 231/2012. ADI “not specified” as |
| | | | classified by the SCF (1991) and WHO Food Additives |
| | | | Series, No. 5 (1985). In a re-evaluation of silicon dioxide (E |
| | | | 551) as a food additive by EFSA (2017), the panel were not |
| | | | able to confirm the current ADI “not specified” due to |
| | | | limitations in available data. |
| Povidone 90F | 50.000 | 4.902 | Povidone 90F may be included at a higher level of 50 mg/mL |
| | | | to impart viscosity to reduce rates of sedimentation to help |
| | | | improve longer term stability. |
| | | | Povidone 90F has been included at levels required to increase |
| | | | viscosity to aid in longer term physical stability, but to remain |
| | | | as low as possible and remaining under the ADI for the |
| | | | anticipated dose adjustment for a given age group. |
| | | | Povidone 90F (Polyvinylpyrrolidone (PVP): The JECFA has |
| | | | granted an ADI of 0-50 mg/kg/day for PVP (Further details |
| | | | in PDCSP) |
| | | | The WHO weight-for-age charts* indicate the body-weight |
| | | | for a 3 month old female child in the 3^{rd} percentile is 4.6 kg |
| | | | equivalent to an ADI for PVP of 230 mg/day. For ages of 3 |
| | | | months-6 months a predicted dose of 18-23 mg API is |
| | | | anticipated. For the 40 mg/mL formulation, this would be |
| | | | equivalent to ~0.5 mL/dose. Assuming 1 dose/day, the oral |
| | | | intake of PVP would be 25 mg which is under the ADI for this |
| | | | age group. |
| | | | The WHO weight-for-age charts indicate the body-weight for |
| | | | a 10 year old female child in the 3^{rd} percentile is 23.5 kg |
| | | | equivalent to an ADI for PVP of 1175 mg/day. For ages 12 |
| | | | year-17 years a predicted dose of 167 mg API is anticipated. |
| | | | For a “worst case scenario” for calculating the intake of PVP |
| | | | if a full 200 mg adult dose would be provided at this age group, |
| | | | for the 40 mg/mL formulation this would be equivalent to a |
| | | | 5 mL dose. Assuming 1 dose/day, the oral intake of PVP |
| | | | would be 250 mg which is under the ADI for this age group |
| Tartaric Acid | Q.s. to pH | Q.s. to pH | Drifts in pH are seen at higher pH values down to ~pH 2.8. |
| | 3.0 ± 0.5 | 3.0 ± 0.5 | No change in XRPD was seen for batches used previously |
| | | | adjusted with tartaric acid that showed a reduction in pH over |
| | | | time. |
| | | | It is proposed tartaric acid may be used to adjust to an |
| | | | initial target pH of pH 3.0 ± 0.5; in another embodiment, |
| | | | to pH 3.0 ± 0.2. |
| | | | In 1990, the Scientific Committee for Food (SCF, now EFSA) |
| | | | established a group ADI of 30 mg/kg body weight for L(+)- |
| | | | tartaric acid (E 334) and its potassium and sodium salts (E |
| | | | 335-337, 354). In a new evaluation the EFSA decided that the |
| | | | available data on systemic bioavailability were robust enough |
| | | | to increase the group ADI to 240 mg/kg body weight (as |
| | | | tartaric acid). |
| Sterile Water | To 1 mL | To 100 | Diluent |
| for Irrigation | | | |
| Total | 1020 | 100.000 | — |

### Example 11: Taste and Bioavailability Studies

Following development of the liquid formulation comprising belumosudil as previously described, studies were performed to (i) evaluate the taste attributes (smell, sweetness, bitterness, flavor, mouth feel/texture, grittiness and aftertaste) and overall acceptability of the liquid formulation comprising belumosudil; (ii) determine the relative bioavailability of the liquid formulation comprising belumosudil in comparison with the belumosudil oral tablet in the fed state; and (iii) determine the effect of food on the PK of belumosudil following administration of the liquid formulation comprising belumosudil.

The first part of this study involved a “sip and spit” taste assessment to profile the taste characteristics of the formulation and identify a suitable flavor system. The outcomes from the taste study were then taken into consideration for the next parts for relative bioavailability assessments of the liquid formulation compared to the oral tablet. For this study, a 48.496 mg/mL belumosudil liquid formulation (40 mg/mL freebase) was used. This concentration was selected to minimize the volume administered in later relative bioavailability studies conducted in healthy volunteers (5 mL to administer a 200 mg dose) and for ease of comparison with the reference tablet (200 mg).

### (a) Part 1—Taste Study

The aim of this first study was to identify an optimal flavor and sweetener combination to facilitate or improve palatability of the liquid formulation of belumosudil for use in orally administering the drug to the target population of patients aged 3 months to 12 years.

### (i). Methods

Twelve healthy, adult male subjects (mean age 35.3 years, min 23, max 52) were enrolled into the taste profile part of the study. Subjects were resident from the day prior to product administration (Day 1) and discharged on Day 2. On Day 1, subjects were randomized to receive a total of six liquid formulations comprising belumosudil pursuant to 1 of 6 treatment sequences (ABFCED, BCADFE, CDBEAF, DECFBA, EFDACB and FAEBDC), with two subjects assigned to each treatment sequence.

Each individual treatment (e.g., A, B, C, D, etc.), involved a single 40 mg/mL (200 mg in 5 mL) oral dose of belumosudil. Bottle formulations and 6 different regimen vehicles were used as follows: A=sterile water (Vehicle 1), B=low sucralose (Vehicle 2), C=high sucralose (Vehicle 3), D=Orange low sucralose (Vehicle 4), E=Tropical fruit blend low sucralose (Vehicle 5), and F=Lemon low sucralose (Vehicle 6).

Prior to the first administration of a liquid formulation comprising belumosudil (either Day 1 or prior to completion of breakfast [pre-dose] on Day 1), subjects were given a training questionnaire using an example fluid (e.g. orange juice/squash). Subjects received the test belumosudil in Bottle regimens according to the randomization schedule.

Subjects received the first single oral dose of their regimen 2 h after consuming a standard breakfast on the morning of Day 1. Each regimen followed the same study design: subjects received a single dose of liquid formulation of belumosudil in bottle, which was held in the mouth for approximately 1 min before being expectorated.

Immediately after expectoration, subjects completed a questionnaire individually and privately to rate the overall acceptability considering seven key taste characteristics (smell, sweetness, bitterness, flavor, mouthfeel/texture, grittiness and aftertaste), with use of a 9-point Likert scale. Specifically, ratings according to this scale as are follows: 1=dislike extremely, 2=dislike very much, 3=dislike moderately, 4=dislike slightly, 5=neither like nor dislike, 6=like slightly, 7=like moderately, 8=like very much, and 9=like extremely.

This process was completed for each of the 6 regimens. No belumosudil was swallowed. There was a washout of approximately 30 min between tasting each regimen (inclusive of palate cleansing). During this time, subjects cleansed their palates using tap water (administered freely in 50 mL aliquots) and crackers before further tasting. All regimens were tasted on the same day.

A single plasma PK sample was taken approximately 1 h post-final dose (prior to discharge from the clinical unit). This sample was retained for analysis only in the case of accidental swallowing of the formulation by a subject and/or for the purpose of investigating a treatment-emergent adverse event (TEAE) considered to be related to the IMP. No subjects reported accidentally swallowing the formulation and/or a TEAE considered to be related to the IMP in Part 1 of the study; therefore, the plasma PK sample was not required to be analyzed for any subject, and was destroyed.

Subjects remained on site until 1 h post-final taste/palatability assessment. To ensure the ongoing well-being of the subjects, a follow-up phone call took place 3 to 7 days post-final dose.

### (ii) Results

The median (min−max) scores for each taste/palatability attribute are summarized in Table 34. Median difference data is summarized in Table 35.

TABLE 34
Min-Max Scores for Each Attribute in Part I Taste
Study for Belumosudil Oral Liquid Formulation

| | Test Treatment | | | | | |
|---|---|---|---|---|---|---|
| | (Regimen) | | | | | |
| | | | | | Tropical | |
| | | | | Orange | fruit | Lemon |
| | Sterile | Low | High | low | blend low | low |
| | Water | Sucralose | Sucralose | sucralose | sucralose | sucralose |
| | (A) | (B) | (C) | (D) | (E) | (F) |
| | Vehicle 1 | Vehicle 2 | Vehicle 3 | Vehicle 4 | Vehicle 5 | Vehicle 6 |
| | Median | Median | Median | Median | Median | Median |
| Taste | Score | Score | Score | Score | Score | Score |
| Attribute | (Min-Max) | (Min-Max) | (Min-Max) | (Min-Max) | (Min-Max) | (Min-Max) |
| Smell | 5.0 | 5.0 | 5.0 | 6.0 | 7.0 | 6.0 |
| | (4-6) | (5-8) | (5-8) | (5-8) | (5-8) | (5-8) |
| Sweetness | 4.5 | 6.0 | 6.5 | 7.0 | 7.0 | 8.0 |
| | (1-6) | (3-8) | (5-9) | (5-9) | (5-9) | (5-8) |
| Bitterness | 4.0 | 5.0 | 5.0 | 5.0 | 5.0 | 5.5 |
| | (1-6) | (2-8) | (3-8) | (4-8) | (5-8) | (2-8) |
| Flavour | 3.5 | 6.0 | 6.0 | 6.5 | 7.0 | 7.0 |
| | (1-5) | (4-7) | (4-8) | (4-9) | (4-9) | (6-9) |
| Mouth feel/ | 3.0 | 4.5 | 4.0 | 4.5 | 5.0 | 5.0 |
| Texture | (1-6) | (2-7) | (1-8) | (1-8) | (2-9) | (1-6) |
| Grittiness | 3.5 | 4.0 | 4.0 | 4.0 | 4.5 | 5.5 |
| | (1-6) | (2-7) | (1-8) | (1-8) | (3-9) | (2-7) |
| Aftertaste | 4.0 | 4.5 | 5.0 | 5.5 | 6.0 | 6.0 |
| | (1-6) | (2-7) | (1-6) | (1-8) | (2-9) | (3-7) |
| Overall | 3.0 | 6.0 | 6.0 | 6.5 | 7.0 | 7.0 |
| | (1-6) | (3-8) | (2-8) | (2-8) | (4-8) | (4-8) |

TABLE 35
Median Difference Results of Part I Taste Study
for Belumosudil Oral Liquid Formulation

| | | | Median Difference | | |
|---|---|---|---|---|---|
| | | | Overall | | |
| Regimen | Sucralose | Flavor | Acceptability | Sweetness | Flavor |
| A | — | — | — | — | — |
| B | Low | — | 2.5 | 2 | 3 |
| C | High | — | 2 | 2 | 3 |
| D | Low | Orange | 2.5 | 2 | 3.5 |
| E | Low | Tropical | 3.5 | 2.5 | 4 |
| F | Low | Lemon | 3 | 3.5 | 3.5 |

Vehicle 1 (sterile water—reference) was the lowest scoring vehicle, with a median score for overall acceptability of 3.0, indicating that the subject panel moderately disliked the product. The flavored regimens (Vehicles 4 [orange low sucralose], 5 [tropical fruit blend low sucralose] and 6 [lemon low sucralose]) achieved median scores of 6.5, 7.0 and 7.0, respectively, indicating that addition of flavoring improved the overall taste/palatability profile of the IMP.

The taste/palatability attributes with the lowest median scores for Vehicle 1 were flavor, mouth feel/texture and grittiness (3.5, 3.0 and 3.5, respectively), indicating that these were the drivers for the dislike. With the addition of a flavored low sucralose vehicle (Vehicles 4, 5 and 6), the median scores for each of these attributes increased, ranging from 6.5 to 7.0 for flavor, 4.5 to 5.0 for mouth feel/texture and 4.0 to 5.5 for grittiness. Median scores also improved for all other taste/palatability attributes with the addition of a flavored low sucralose vehicle, indicating the subjects preferred the taste profiles of these vehicles.

For each taste attribute, the Friedman's Test was statistically significant at the 5% significance level indicating that at least one of the formulations had a significantly different taste score to the other treatments (p<0.001 for overall acceptability, smell, sweetness, bitterness, flavor and mouth feel/texture, p=0.041 and p=0.001 for grittiness and aftertaste, respectively).

For all pairwise comparisons and taste attributes, the median of the paired differences was positive indicating that use of a sweetener and/or flavor showed improved acceptability compared to the reference vehicle, sterile water.

In all taste attributes, the tropical fruit blend low sucralose or the lemon low sucralose vehicles showed either the greatest improvement or joint greatest improvement compared to the reference vehicle (based on median of paired differences). There was no benefit demonstrated to increasing sucralose content of the vehicle.

### (b) Part 2—Bioavailability

### (i) Methods

Part 2 was a single centre, open label, randomized, three-period design to assess the relative bioavailability of selected belumosudil liquid formulations compared to the belumosudil oral tablet and the effect of food on the liquid formulation comprising belumosudil in 18 healthy male subjects. Subjects underwent preliminary screening procedures to determine their eligibility for Part 2 of the study at the screening visit (Day −28 to Day −2 of Part 2). Subjects who took part in Part 1 of the study were permitted to take part in Part 2.

Subjects received single oral doses of 200 mg belumosudil over 3 periods. Three different regimens (G, H, and I), were used over the three periods. In Regimen G, belumosudil was administered in Tablet form (reference); and in Regimens H and I, belumosudil was administered in the form of Oral Liquid Formulation.

In Period 1, Day 1: subjects were randomized prior to administration of the first dose of IMP to 1 of 6 treatment sequences (GHI, HIG, IGH, IHG, GIH and HGI) with 3 subjects assigned to each treatment sequence. On Day 1 of each study period (periods 1, 2 and 3), subjects received the regimens in Table 36:

TABLE 36
Regimens Used in Bioavailability/Follow on Taste Study

| | | | | Route of |
|---|---|---|---|---|
| Period | Regimen | IMP | Dose^{a} | Administration |
| 1, 2 and 3 | G | Belumosudil | 200 mg | Oral, Fed |
| (randomised) | | Tablet (reference) | | |
| | H | Belumosudil | 200 mg | Oral, Fasted |
| | | Liquid Formulation | | |
| | I | Belumosudil | 200 mg | Oral, Fed |
| | | Liquid Formulation | | |

Regimen H was administered to subjects as a flavorless low sucralose liquid formulation in a fasted state, and Regimens G and I were administered to subjects in a fed state. Regimen G was administered with a total of 240 mL of water. For Regimens H and I, immediately after IMP administration, subjects consumed water to a total volume of 240 mL (including the dosing volume).

Each study period followed a similar design. Subjects were admitted to the clinical unit on the morning before the first IMP administration (Day −1 of Period 1) for confirmation of eligibility and baseline procedures. For Regimen H only, subjects were given a training questionnaire using an example fluid (e.g. orange juice/squash) prior to IMP administration to demonstrate how the questionnaire should be completed. This was performed either on Day −1 (only applicable if Regimen H was administered in Period 2 or 3) or prior to dosing (pre-dose) on Day 1. The flavor system determined to be the most favorable from Part 1 (lemon flavor with low sucralose solution) was originally planned to be used in Regimen H in Part 2, however, a flavorless low sucralose oral liquid formulation was used instead.

Subjects received a single dose of IMP in the morning of Day 1 following an overnight fast of a minimum of 10 h (Regimen H, fasted state) or following a standard breakfast (Regimens G and I, fed state). Blood samples were collected at regular intervals for PK analysis. Following administration of Regimen H, subjects completed a written taste/palatability questionnaire individually and privately.

Subjects were resident in the clinical unit for 10 consecutive nights that covered all 3 treatment periods. All subjects remained on site until 72 h post-final dose for safety and PK assessments. There was a minimum washout of 3 days between each IMP administration. To ensure the ongoing wellbeing of the subjects, a follow-up phone call took place 3 to 7 days post-final dose. If a subject reported any AEs after discharge which represented a cause for concern, they were required to attend the clinical unit for a follow-up assessment. This would have been an unscheduled visit.

### (ii) Results

a. Taste/Palatability

Overall, the oral liquid formulation (Regimen H) was rated either Grade 5 (‘Neither Like Nor Dislike’), Grade 6 (‘Like Slightly’) or Grade 7 (‘Like Moderately’) by the majority of subjects. The overall taste profile scores for Regimen H were comparable to those for Regimen B in Part 1, which utilized the same sweetener combination (i.e. low sucralose).

b. Bioavailability

Following single oral administration of the liquid formulation comprising belumosudil, absorption of belumosudil was faster (median Tmax 2 h) compared to belumosudil administered as the tablet reference (median Tmax 3 h).

Belumosudil was administered as a reference tablet in the fed state. Belumosudil was absorbed with median Tmax occurring at 3.00 h post-dose following which concentrations exhibited a rapid decline giving rise to a geometric mean T1/2 of 9.393 h, similar to those observed previously. The inter-subject variability associated with exposure (Cmax and AUC) was moderate at 26.1%-39.6%. Median Tmax of KD025 m1 and KD025m2 following administration of belumosudil tablet was like that of belumosudil at 2.00 h and 3.00 h post-dose, respectively. Concentrations of KD025 ml were sparsely quantifiable and terminal half-lives were only reliably estimated for 2 subjects. Geometric mean T1/2 of KD025m2 was 2.466 h, estimated reliably in 8 out of the 18 subjects. Inter-subject variability associated with exposure was higher for both metabolites compared to parent, at 37.9%-63.9% and 52.5%-75.3% for KD025 ml and KD025m2, respectively.

The key geometric mean (geometric coefficient of variation [CV %]) PK parameters for plasma belumosudil following dosing with belumosudil are summarized below in Table 37.

TABLE 37
Comparative PK Parameters for Plasma Belumosudil for
Tablet versus Liquid Formulation Administration

| | Regimen Dose Level Status | | | | | |
|---|---|---|---|---|---|---|
| | | | H | | I | |
| | G | | 200 mg Liquid | | 200 mg Liquid | |
| | 200 mg | | Formulation | | Formulation | |
| | Tablet Fed | | Fasted | | Fed | |
| | No. of Subjects | | | | | |
| Parameter | N = 18 | | N = 18 | | N = 18 | |
| Tlag^{a} (h) | 0.500 | (0.00-2.00) | 0.00 | (0.00-0.00) | 0.00 | (0.00-0.00) |
| Tmax^{a} (h) | 3.00 | (1.00-5.00) | 1.50 | (1.00-4.00) | 2.00 | (1.00-3.03) |
| Cmax (ng/mL) | 1930 | (26.1%) | 1550 | (33.3%) | 1790 | (25.1%) |
| AUC (0-24) | 9310 | (37.5%) | 7620 | (44.9%) | 9340 | (34.6%) |
| (ng · h/mL) | | | | | | |
| AUC (0-last) | 9720 | (39.3%) | 8010 | (48.4%) | 9570 | (37.6%) |
| (ng · h/mL) | | | | | | |

| | | | |
|---|---|---|---|
| AUC (0-inf) | 10200 (39.6%) | 8430 (46.4%) | 9500 (40.9%) |
| (ng · h/mL) | [n = 16] | [n = 13] | [n = 15] |
| AUC % | 2.601 (44.8%) | 2.785 (50.4%) | 1.918 (34.9%) |
| extrap (%) | [n = 16] | [n = 13] | [n = 15] |
| T½ (h) | 9.393 (57.7%) | 9.499 (43.5%) | 6.788 (49.2%) |
| | [n = 16] | [n = 13] | [n = 15] |
| Lambda-z (l/h) | 0.074 (57.7%) | 0.073 (43.5%) | 0.102 (49.2%) |
| | [n = 16] | [n = 13] | [n = 15] |
| CL/F (mL/min) | 326 (39.6%) | 395 (46.4%) | 351 (40.9%) |
| | [n = 16] | [n = 13] | [n = 15] |
| Vz/F (L) | 265 (62.6%) | 325 (47.9%) | 206 (27.6%) |
| | [n = 16] | [n = 13] | [n = 15] |

n: number of subjects with an observation;
N: number of subjects in the dataset;
NC: not calculated
^{a}Medium (range)

Following a single oral administration of belumosudil to healthy male volunteers as a tablet reference in the fed state (Regimen G), concentrations of belumosudil were evident from between 0.5 h and 3 h in all subjects. Following dosing with a liquid formulation comprising belumosudil in the fasted and fed states (Regimens H and I), concentrations were evident from 0.5 h in all subjects. Maximum plasma concentrations occurred between 1 h and 5 h post-dose.

Concentrations then declined in a biphasic manner and remained quantifiable for up to between 24 h and 72 h post-dose. Resultant elimination half-lives ranged between 4.84 h and 33.02 h, 5.36 h and 23.76 h and 2.38 h and 15.73 h, respectively. The geometric mean half-life ranged from 6.788 h to 9.499 h.

The key geometric mean (geometric coefficient of variation [CV %]) PK parameters for plasma KD025 ml and KD025m2 following dosing with belumosudil are summarized below in Tables 38 and 39, respectively.

TABLE 38
Comparative PK Parameters for Plasma KD025m1 for
Tablet versus Liquid Formulation Administration

| | Regimen Dose level Status | | | |
|---|---|---|---|---|
| | | | H | I |
| | G | | 200 mg Liquid | 200 mg Liquid |
| | 200 mg | | Formulation | Formulation |
| | Tablet Fed | | Fasted | Fed |
| | No. of Subjects | | | |
| Parameter | N = 18 | | N = 18 | N = 18 |
| Tlag^{a} (h) | 1.00 | (0.500-2.00) | 0.00 (0.00-1.00) [n = 17] | 0.500 (0.00-1.00) [n = 17] |
| Tmax^{a} (h) | 2.00 | (1.00-4.03) | 1.00 (0.500-1.50) [n = 17] | 1.50 (0.500-3.00) [n = 17] |
| Cmax (ng/mL) | 22.3 | (37.9%) | 19.0 (41.7%) [n = 17] | 19.4 (27.6%) [n = 17] |
| AUC(0-24) | 45.3 (63.9%) [n = 15] | | 34.9 (104.8%) [n = 12] | 49.4 (83.1%) [n = 17] |
| (ng · h/mL) | | | | |
| AUC(0-last) | 42.7 (50.7%) [n = 15] | | 30.3 (70.9%) [n = 12] | 37.9 (53.2%) [n = 17] |
| (ng · h/mL) | | | | |
| T½ (h) | 1.81, 2.88 [n = 2] | | 2.28, 2.67 [n = 2] | 2.128 [n = 1] |
| Lambda-z (l/h) | 0.24, 0.38 [n = 2] | | 0.26, 0.30 [n = 2] | 0.326 [n = 1] |
| MPR Cmax | 0.013 | (40.6%) | 0.014 (36.0%) [n = 17] | 0.012 (31.7%) [n = 17] |

| | | | |
|---|---|---|---|
| MPR AUC(0-24) | 0.005 (62.0%) [n = 15] | 0.005 (87.7%) [n = 12] | 0.006 (78.3%) [n = 17] |
| MPR AUC(0-last) | 0.004 (52.1%) [n = 15] | 0.004 (59.9%) [n = 12] | 0.004 (49.9%) [n = 17] |

TABLE 39
Comparative PK Parameters for Plasma KD025m2 for
Tablet versus Liquid Formulation Administration

| | Regimen Dose level Status | | | | | |
|---|---|---|---|---|---|---|
| | | | H | | I | |
| | G | | 200 mg Liquid | | 200 mg Liquid | |
| | 200 mg | | Formulation | | Formulation | |
| | Tablet Fed | | Fasted | | Fed | |
| | No. of Subjects | | | | | |
| Parameter | N = 18 | | N = 18 | | N = 18 | |
| Tlag^{a} (h) | 1.00 | (0.500-2.00) | 0.00 | (0.00-0.00) | 0.00 | (0.00-1.00) |
| Tmax^{a} (h) | 3.00 | (1.50-5.00) | 1.50 | (1.00-3.02) | 2.00 | (1.00-3.03) |
| Cmax (ng/mL) | 406 | (52.5%) | 372 | (56.4%) | 319 | (57.9%) |
| AUC(0-24) | 1280 | (62.9%) | 1130 | (61.8%) | 1180 | (58.1%) |
| (ng · h/mL) | | | | | | |
| AUC(0-last) | 1200 | (65.7%) | 1080 | (62.2%) | 1120 | (59.5%) |
| (ng · h/mL) | | | | | | |
| AUC(0-inf) | 1180 (75.3%) [n = 8] | | 1030 (59.8%) [n = 10] | | 926 (56.0%) [n = 10] | |
| (ng · h/mL) | | | | | | |
| AUC % extrap (%) | 5.607 (49.8%) [n = 8] | | 4.453 (60.7%) [n = 10] | | 5.388 (43.1%) [n = 10] | |
| T½ (h) | 2.466 (43.1%) [n = 8] | | 2.506 (87.4%) [n = 10] | | 2.539 (56.5%) [n = 10] | |
| Lambda-z (1/h) | 0.281 (43.1%) [n = 8] | | 0.277 (87.4%) [n = 10] | | 0.273 (56.5%) [n = 10] | |
| MPR Cmax | 0.232 | (42.1%) | 0.264 | (44.1%) | 0.196 | (45.6%) |
| MPR AUC(0-24) | 0.151 | (40.2%) | 0.164 | (45.1%) | 0.139 | (37.7%) |
| MPR AUC(0-last) | 0.136 | (42.6%) | 0.149 | (47.0%) | 0.129 | (38.1%) |

| | | | |
|---|---|---|---|
| MPR AUC(0-inf) | 0.136 (42.7%) [n = 7] | 0.139 (49.0%) [n = 6] | 0.117 (40.2%) [n = 7] |

Administration of a liquid formulation comprising belumosudil in the fasted state showed an earlier median Tmax (1.5 h) compared to the same dose in the fed state.

Comparison of the GMRs relating to peak (Cmax) and total exposure (AUC(0-last) and AUC(0-inf)) levels for belumosudil liquid formulation in the fed state vs tablet in the fed state indicated that levels of peak and total exposure to belumosudil and KD025 ml for the liquid formulation were largely similar to that of the tablet reference, with the 90% CI for each parameter including 100%. The upper bound of the 90% CI for peak exposure was marginally below 100% (upper CI 94.39%); although it did exclude unity, this indicates that any true difference could be minimal and unlikely to be of clinical significance. Additionally, the change in formulation resulted in little to no change in AUC, indicating change in formulation had no notable impact on overall exposure to belumosudil or its two known metabolites.

Comparison of the GMRs relating to peak and total exposure for oral liquid formulation fed vs fasted demonstrated a slight increase in for belumosudil after the fed regimen, averaging 15% to 19% greater exposure than that seen for the fasted regimen. For KD025 ml, moderate increases in total exposure were observed, although this increase should be interpreted with caution due to the relatively sparse data available for KD025 ml parameter estimation across all regimens. The lower bound of the 90% CI for each parameter exceeded 100%. No notable changes based on food effect were observed for KD025m2.

In summary, following single oral administration of the liquid formulation comprising belumosudil, absorption of belumosudil was faster (median Tmax 2 h) compared to belumosudil administered as the Tablet reference (median Tmax 3 h).

The median Tmax of KD025 ml and KD025m2 was similarly faster following administration as a liquid formulation (1.5 h and 2 h, respectively) compared to the Tablet reference (2 h and 3 h, respectively).

Maximum (Cmax) and overall (AUC) belumosudil exposure following administration as an oral liquid formulation showed no change following change in formulation compared to the Tablet reference, both in the fed state.

Bioavailability, as measured by Cmax and AUC, was similar for belumosudil and metabolites (KD025 ml and KD025m2) between the liquid formulation and tablet.

### Example 12: Pediatric Dose Studies

In this study, recommended pediatric dosages for belumosudil matching the AUC following a 200 mg once daily (QD) dose of belumosudil in adults were estimated using two models, a Population Pharmacokinetic (PopPK) model and a Physiologically Based Pharmacokinetic (PBPK) model.

### (a) PopPK Model

In applying this model, the following steps were taken: (a) body weight bins were decided for pediatric and adolescent body weights between 10 kg (3rd percentile for 2-year-olds) and 88 kg (97th percentile for 18-year-olds); (b) predictions were made over a 24 h period at steady state; (c) the impact of GVHD on clearance was accounted for in each subject; (d) it was assumed that PPIs were not co-administered for any virtual subject; (e) each population was assigned 50% males; and (f) the final exposure predictions were compared to a virtual adult group wherein all adults were administered 200 mg KD025 QD.

In identifying appropriate body weight bins, the following steps were taken: (a) the pediatric and adolescent body weight range (10 kg to 88 kg) was divided into bins of 3 kg with 1000 virtual subjects within the body weight range in each bin; (b) AUC was predicted in subjects following 50 mg, 100 mg, and 200 mg QD doses; (c) the AUC was predicted in an adult population of 5000 virtual subjects following a 200 mg QD dose; (d) the 25th percentile AUC in adults was compared to 50th percentile AUC in each weight bin, and bins with higher median AUC were combined into one larger weight bin for each dose.

Adult body weights range from 40 kg to 126 kg. The following body weight bins were used for final exposure predictions to align dosing with adults and for ease of dosing:

$$10 kg to < 20 kg - 50 mg QD$$

$$20 kg to < 40 kg - 100 mg QD$$

$$\geq 40 kg - 200 mg QD$$

Applying this model, the final predicted weight bins and doses were determined to be: (1) for patients weighing 10 kg to <19 kg-50 mg QD; (2) for patients weighing 19 kg to <49 kg-100 mg QD; and (3) for patients weighing 49 kg to <88 kg-200 mg QD. The estimated once daily (QD) and twice daily (BID) dosages for belumosudil oral liquid formulation by age group and body weight are set forth in more detail below in Tables 40 to 43.

TABLE 40
Estimated Pediatric QD Dose for Belumosudil by Age Group

| | Minimum Body | Maximum Body | Default |
|---|---|---|---|
| Age Group | Weight (kg) | Weight (kg) | (mg) |
| 3 m to <6 m | 6.286 | 8.18 | 24 |
| 6 m to <12 m | 7.967 | 10.85 | 35 |
| 12 m to <24 m | 10.29 | 13.43 | 48 |
| 24 m to <72 m | 12.86 | 22.41 | 69 |
| (>2 but <6 y) | | | |
| 72 m to <144 m | 22.32 | 46.96 | 110 |
| (>6 but <12 y) | | | |
| 144 m to <216 m | 45.91 | 74.17 | 173 |
| (>12 but <18 y) | | | |

TABLE 41
Estimated Pediatric QD Dose for Belumosudil by Body Weight

| Body Weight | Minimum Age | Maximum Age | Default |
|---|---|---|---|
| Group | (months) | (months) | (mg) |
| 6 kg to <8 kg | 3.002 | 6.998 | 24 |
| 8 kg to <10 kg | 5.012 | 11.97 | 33 |
| 10 kg to <13 kg | 9.007 | 24.95 | 45 |
| 13 kg to <20 kg | 21.01 | 61.9 | 64 |
| 20 kg to <40 kg | 60.02 | 131 | 97 |
| >=40 kg | 127.1 | 216 | 167 |

TABLE 42
Estimated Pediatric BID Dose for Belumosudil by Age

| | Minimum Body | Maximum Body | Default |
|---|---|---|---|
| Age Group | Weight (kg) | Weight (kg) | (mg) |
| 3 m to <6 m | 6.286 | 8.176 | 12 |
| 6 m to <12 m | 7.967 | 10.85 | 17 |
| 12 m to <24 m | 10.29 | 13.43 | 24 |
| 24 m to <72 m | 12.86 | 22.41 | 34 |
| (>2 but <6 y) | | | |
| 72 m to <144 m | 22.32 | 46.96 | 55 |
| (>6 but <12 y) | | | |
| 144 m to <216 m | 45.91 | 74.17 | 87 |
| (>12 but <18 y) | | | |

TABLE 43
Estimated Pediatric BID Dose for Belumosudil by Body Weight

| | Minimum Age | Maximum Age | Default |
|---|---|---|---|
| Body Weight Group | (months) | (months) | (mg) |
| 6 kg to <8 kg | 3.002 | 6.998 | 12 |
| 8 kg to <10 kg | 5.012 | 11.97 | 16 |
| 10 kg to <13 kg | 9.007 | 24.95 | 22 |
| 13 kg to <20 kg | 21.01 | 61.9 | 32 |
| 20 kg to <40 kg | 60.02 | 131 | 48 |
| >=40 kg | 127.1 | 216 | 83 |

### (b) PBPK Model

Physiologically based pharmacokinetic (PBPK) models for belusmosudil (KD025) and its major metabolite, KD025m2 (having the chemical name 2-(3-(4-(1H-indazol-5-ylamino)quinazolin-2-yl)phenoxy)acetic acid) (also, “M2”), were developed previously by incorporating data from in vitro, nonclinical and clinical pharmacokinetic studies in healthy volunteers. See, e.g., Schueller, O., Et Al., “A Phase I Pharmacokinetic Drug Interaction Study of Belumosudil Coadministered With CYP3A4 Inhibitors and Inducers and Proton Pump Inhibitors,” Clinical Pharmacology in Drug Development, 2022, 11 (7) 795-806.

In this Example, previously developed PBPK models for KD025 and M2 were applied to predict the systemic exposure of KD025 in children (3 months-11 years) and adolescents (12-17 years) with the aim of supporting dose selection.

The developed PBPK models were applied with the following four steps:
1. Plasma concentrations of KD025 and M2 in healthy adult subjects were predicted following multiple oral doses of 2.7 mg/kg QD KD025 (200 mg equivalent adult dose).
2. Plasma concentrations of KD025 and M2 in healthy pediatric subjects were predicted (for age groups of 0.25-0.5 y, 0.5-1 y, 1-2 y, 2-5 y, 6-11 y and 12-17 y) following multiple oral doses of 2.7 mg/kg QD KD025.
3. The initial 2.7 mg/kg dose of KD025 was adjusted based on simulated pediatric: adult KD025 AUC_{0-t,SS} ratios (calculated from steps 1 and 2).
4. Plasma concentrations of KD025 and M2 in pediatric subjects were predicted (for age groups of 0.25-0.5 y, 0.5-1 y, 1-2 y, 2-5 y, 6-11 y and 12-17 y) following multiple oral doses of the adjusted dose of KD025.

For comparison, allometric scaling was also investigated based on body weight to predict KD025 dose in infants and children (0.25-0.5 y, 0.5-1 y, 1-2 y, 2-5 y, 6-11 y and 12-17 y) using adult daily doses of 200 mg.

Step 1. Simulation of Plasma Concentration-Time Profiles of KD025 and M2 in Healthy Adults after Multiple Oral Doses of KD025 (2.7 mg/Kg QD, Equivalent to 200 mg QD)

Ten virtual trials of 20 healthy adult subjects (50% female) aged 20 to 50 years were generated using the Sim-Healthy Volunteer population library. The ‘Simcyp Paediatric Simulator’ allows pharmacokinetic behavior to be modelled in neonates, infants and children. The Simcyp Simulator includes a full PBPK model together with extensive libraries on paediatric demography (age, height, weight, BSA [body surface area]), developmental physiology (liver size, renal function, liver blood flow) and biochemistry (albumin, CYP ontogeny). The algorithms describing these changes are described in the literature. (See, e.g., Johnson and Rostami-Hodjegan, Resurgence in the Use of Physiologically Based Pharmacokinetic Models in Pediatric Clinical Pharmacology, Pediatr Anesth. 21:291-301 [2011]).

The adult dose of interest (200 mg) was converted to a dose of 2.7 mg/kg using a simulated mean adult body weight of 73.84 kg. The virtual subjects received multiple oral doses of 2.7 mg/kg KD025 QD for 8 days and simulated individual values for AUC_{0-t,SS} were integrated on Day 8 (simulation hours 168-192).

Mean simulated plasma KD025 and M2 concentrations following multiple oral dosing of 200 mg QD KD025 for 8 days were calculated. The predicted mean Cₘₐₓ and AUC values for KD025 and M2 on Day 8 are shown in Table 44.

TABLE 44
Predicted geometric mean Cₘₐₓ, tₘₐₓ and AUC values
for KD025 and M2 in healthy adult subjects.

Day 8

| | tₘₐₓ | Cₘₐₓ | AUC |
|---|---|---|---|
| | (hr) | (ng/mL) | (hr * ng/mL) |
| KD025 GM | 2.25 | 1796 | 7907 |
| 90% CI | (1.26 4.35) | (1697-1902) | (7540-8293) |
| M2 GM | 2.35 | 300 | 1060 |
| 90% CI | (1.50-4.55) | (287-314) | (1016-1105) |

Step 2. Simulation of Plasma Concentration-Time Profiles of KD025 and M2 in Paediatric Subjects (3 Months-17 Years) after Multiple Oral Doses of KD025 (2.7 mg/Kg QD).

The pediatric subjects were separated into the following age bands; 0.25-0.5 y, 0.5-1 y, 1-2 y, 2-5 y, 6-11 y and 12-17 y. Ten virtual trials of 20 pediatric subjects (50% female) for each age band receiving 2.7 mg/kg QD (200 mg adult equivalent) of KD025 were generated using the default Sim-Paediatric population library. Simulations were performed using the CYP3A4 Ontogeny profile of Upreti and Wahlstrom, Meta-Analysis of Hepatic Cytochrome P450 Ontogeny to Underwrite the Prediction of Pediatric Pharmacokinetics Using Physiologically Based Pharmacokinetic Modelling, J Clin Pharmacol. 56:266-283 (2016).

The virtual subjects received multiple oral daily doses of KD025 for 8 days and simulated individual values for AUC_{0-t,ss} were integrated on Day 8 (simulation hours 168-192). The equivalent doses in units of mg are specified below, for each age group, in Table 43.

The pediatric subjects were separated into the following age bands; 0.25-0.5 y, 0.5-1 y, 1-2 y, 2-5 y, 6-11 y and 12-17 y. Ten virtual trials of 20 pediatric subjects (50% female) for each age band receiving adjusted multiple oral doses KD025 were generated using the default Sim-Pediatric population library. All simulations were performed using the Upreti Wahlstrom 2016 CYP3A4 Ontogeny profile.

The virtual subjects received multiple oral daily doses of KD025 for 8 days and simulated individual values for AUC_{0-t,ss} were integrated on Day 8 (simulation hours 168-192). The adjusted doses and equivalent doses in units of mg are specified for each age group in Table 45.

TABLE 45
Adjusted Doses Used for Each Pediatric Age Group
(Mg/Kg) and Equivalent Doses in Units of Mg.

| | Mean | | | |
|---|---|---|---|---|
| | simulated | Initial | Adjusted | Adjusted |
| | body weight | dose | dose | dose |
| Age (y) | (kg) | (mg) ^{a, b} | (mg/kg) | (mg) ^{b} |
| 0.25-0.5 | 6.6 | 18 | 3.5 | 23 |
| 0.5-1 | 9.0 | 24 | 3.5 | 31 |
| 1-2 | 11.5 | 31 | 3.5 | 40 |
| 2-5 | 15.4 | 42 | 3.6 | 55 |
| 6-11 | 27.6 | 75 | 3.5 | 97 |
| 12-17 | 53.9 | 146 | 3.1 | 167 |

^{a} Equivalent to 2.7 mg/kg
^{b} For reference only (simulations were run using doses in units of mg/kg)

Predicted plasma concentration-time profiles of KD025 and M2 following multiple oral doses of 2.7 mg/kg in pediatrics aged 0.25-0.5 y, 0.5-1 y, 1-2 y, 2-5 y, 6-11 y and 12-17 y using the Upreti Wahlstrom (2016) CYP3A4 ontogeny profiles were calculated using, for each simulation, concentration-time profiles representative of the mean of the total virtual population (n=200). The predicted mean KD025 and M2 AUC_{0-t,SS} values on Day 8 are shown in Table 46.

TABLE 46
Predicted mean plasma AUC_{0-t, SS} values for KD025 and M2 following
multiple oral doses of KD025 (2.7 mg/kg; adult equivalent
200 mg dose QD) in adult and paediatric subjects.

| | Upreti Wahlstrom (2016) | | Upreti Wahlstrom (2016) | |
|---|---|---|---|---|
| | CYP3A4 ontogeny | | CYP3A4 ontogeny | |
| | Geometric | | Geometric | |
| | Mean | KD025 | Mean | M2 |
| | KD025 | AUC_{0-t, SS} | M2 | AUC_{0-t, SS} |
| | AUC_{0-t, SS} | relative | AUC_{0-t, SS} | relative |
| Age (y) | (hr * ng/mL) | to adult | (hr * ng/mL) | to adult |
| 20-50 (adult) | 7907 | 1.00 | 1060 | 1.00 |
| 0.25-0.5 | 6208 | 0.79 | 726 | 0.68 |
| 0.5-1 | 6097 | 0.77 | 751 | 0.71 |
| 1-2 | 5963 | 0.75 | 760 | 0.72 |
| 2-5 | 5835 | 0.74 | 767 | 0.72 |
| 6-11 | 6007 | 0.76 | 762 | 0.72 |
| 12-17 | 6896 | 0.87 | 834 | 0.79 |

Step 3. Adjustment of the Initial 2.7 mg/Kg Dose of KD025 Based on Simulated Paediatric/Adult KD025 AUC_{0-t,SS} Ratios

Pediatric doses were adjusted from the initial value of 2.7 mg/kg based on calculated paediatric/adult KD025 AUC_{0-t,SS} ratios (simulations described in Steps 1 and 2, above) as shown in Equation (1).

$$Adjusted Dose = Initial Dose \cdot \frac{Adult {AUC}_{0 - t , SS}}{Pediatric {AUC}_{0 - t , SS}} \qquad Equation ( 1 )$$

Adjusted doses (mg/kg) were calculated across the following age ranges; 0.25-0.5 y, 0.5-1 y, 1-2 y, 2-5 y, 6-11 y and 12-17 y and are described in Table 43.

Sensitivity analysis was also performed for each paediatric age group at several doses around the calculated adjusted dose (within the range 2.7-3.6 mg/kg) to confirm that application of the adjusted dose resulted in similar KD025 and M2 exposures in both pediatric and adult populations.

Pediatric doses were adjusted from the initial value of 2.7 mg/kg based on calculated paediatric/adult KD025 AUC_{0-t,SS} ratios (simulations described in Steps 1 and 2 and Table 44), using the Upreti Wahlstrom (2016) CYP3A4 ontogeny profile). Adjusted doses are given above in Table 45.

Step 4. Simulation of Plasma Concentration-Time Profiles of KD025 and M2 in Pediatric Subjects (3 Months-17 Years) after Adjusted Multiple Oral Doses of KD025.

The pediatric subjects were separated into the following age bands; 0.25-0.5 y, 0.5-1 y, 1-2 y, 2-5 y, 6-11 y and 12-17 y. Ten virtual trials of 20 paediatric subjects (50% female) for each age band receiving adjusted multiple oral doses KD025 were generated using the default Sim-Paediatric population library. All simulations were performed using the Upreti Wahlstrom 2016 CYP3A4 Ontogeny profile.

The virtual subjects received multiple oral daily doses of KD025 for 8 days and simulated individual values for AUC_{0-t,SS} were integrated on Day 8 (simulation hours 168-192). The adjusted doses and equivalent doses in units of mg are specified for each age group in Table 45.

Predicted plasma concentration-time profiles of KD025 and M2 following dose adjustment in paediatrics aged 0.25-0.5 y, 0.5-1 y, 1-2 y, 2-5 y, 6-11 y and 12-17 y using the Upreti Wahlstrom (2016) CYP3A4 ontogeny profile were calculated including, for each simulation, concentration-time profiles representative of the mean of the total virtual population (n=200). Adjusted doses and associated predicted mean KD025 and M2 AUC_{0-t,SS} values on Day 8 are shown in Table 47.

Simulations were run using dose in units of mg/kg.

TABLE 47
Predicted mean plasma AUC_{0-t, SS} values for KD025 and M2 following
multiple oral doses in paediatric subjects following dose
adjustment using the Upreti Wahlstrom (2016) ontogeny.

| | | | Upreti Wahlstrom (2016) | | Upreti Wahlstrom (2016) | |
|---|---|---|---|---|---|---|
| | | | CYP3A4 ontogeny | | CYP3A4 ontogeny | |
| | | | Geometric | KD025 | Geometric | M2 |
| | | | Mean | AUC_{0-t, SS} | Mean | AUC_{0-t, SS} |
| | Adjusted | Adjusted | KD025 | relative | M2 | relative |
| | Dose | Dose (mg | AUC_{0-t, SS} | to | AUC_{0-t, SS} | to |
| Age (y) | (mg/kg) | equivalent) | (hr*ng/mL) | adult | (hr*ng/mL) | adult |
| 20-50 | 2.7 | 200 | 7907 | 1.00 | 1060 | 1.00 |
| (adult) | | | | | | |
| 0.25-0.5 | 3.5 | 23 | 7999 | 1.01 | 928 | 0.88 |
| 0.5-1 | 3.5 | 31 | 8032 | 1.02 | 966 | 0.91 |
| 1-2 | 3.5 | 40 | 7851 | 0.99 | 980 | 0.92 |
| 2-5 | 3.6 | 55 | 8090 | 1.02 | 1007 | 0.95 |
| 6-11 | 3.5 | 97 | 7883 | 1.00 | 981 | 0.93 |
| 12-17 | 3.1 | 167 | 7964 | 1.01 | 953 | 0.90 |

Allometric scaling was performed based on the body weight (BW) using the three quarter power model as shown in Equation (2).

$${Dose}_{Pediatric} = {Dose}_{Adult} * {( \frac{{BW}_{Pediatric}}{{BW}_{Adult}} )}^{0.75} \qquad Equation ( 2 )$$

Doses were calculated across the following age ranges; 0.25-0.5 y, 0.5-1 y, 1-2 y, 2-5 y, 6-11 y and 12-17 y. The pediatric and adult BW was taken as the mean simulated value from each age range (Table 43).

Allometric dose predictions in pediatric subjects aged 3 months to 17 years using an adult daily dose of 200 mg are provided in Table 48.

TABLE 48
Allometric dose prediction in paediatric
subjects aged 3 months to 17 years

| | Mean | | |
|---|---|---|---|
| | simulated | Pediatric | Pediatric |
| | body weight | Dose | Dose |
| Age (y) | (kg) | (mg) | (mg/kg) |
| 0.25-0.5 | 6.6 | 33 | 5.0 |
| 0.5-1 | 9 | 41 | 4.6 |
| 1-2 | 11.5 | 50 | 4.3 |
| 2-5 | 15.4 | 62 | 4.0 |
| 6-11 | 27.6 | 96 | 3.5 |
| 12-17 | 53.9 | 158 | 2.9 |

Results of simulations performed in this Example using the Upreti Wahlstrom (2016) CYP3A4 ontogeny profile suggest small differences in KD025 and M2 exposure in pediatric subjects in comparison to adults (within 30% of and 1.5-fold of adult AUC_{0-t,SS} for KD025 and M2, respectively) when dosed on a mg per kg basis.

Simple allometric scaling of data may lead to an overestimation of the required dose in youngest age groups (age 5 and under). For the older age groups (age 6-17 years), allometry resulted in dose adjustments that were similar to the Upreti Wahlstrom ontogeny derived PBPK predictions.

### Additional Simulations.

The model was developed with the following assumptions: The drug is administered as a tablet once a day; the drug response is assumed to be dose proportional; the effect of concomitant administration of CYP inducers or inhibitors is not considered; the dose is administered in fed state; all pediatric subjects are assumed to have chronic graft versus host disease (cGVHD); each virtual population has 1000 subjects, 50% of which are males; and the impact of concomitant administration of PPI on drug PK is not considered.

Predicting Pediatric Exposure with Adult Equivalent Mg/Kg Dosing.

Each virtual population spanned a 3 month age range starting at 6 months and ending at 216 months. A flat dose of 2.5 mg/kg—equivalent to 200 mg dose for a 80.65 kg adult—was administered to each subject. The dose was administered QD. The model was run to steady state for exposure predictions. The Cmax at steady state and AUC over 24 h period at steady state were used as exposure metrics. The pediatric exposures were compared to exposures in adults.

Predicting pediatric exposure with simulations based on age-based dosing strategy.

The pediatric population was divided into 3 age bins: 1 Y to 6 Y, 6 Y to 12 Y, and 12 Y to 18 Y. Doses of 3.5, 4, 4.5, and 5 mg/kg QD were administered to 1 Y to 6 Y and 6 Y to 12 Y populations. A dose of 200 mg QD was administered to 12 Y to 18 Y dose group. The model was run to steady state for exposure predictions. The Cmax at steady state and AUC over 24 h period at steady state were used as exposure metrics. The pediatric exposures were compared to exposures in adults.

### Impact of Enzyme Ontogeny on Drug Exposure.

A 4 mg/kg dose was administered to population binned in 3 month age-bins starting from 6 months up to 60 months. The fraction activity for each subject was calculated using the following three ways: Ontogeny only, Allometry at >2 years of age, and Allometry only. The predicted exposures were then binned in 1 kg bodyweight bins to evaluate the need for different bodyweight based dosing. For pediatric patients >=13 kg, ontogeny of CYP enzymes plays has limited impact on drug exposure at 4 mg/kg dose level. For the virtual population, maximum weight of patients within the 18 m to 21 m age group was 13 kg.

### Predicting Pediatric Exposure Based on Body Weight Bins.

The 3 mg/kg, 3.5 mg/kg, and 4 mg/kg dose was administered to population binned in 2-month age-bins starting from 6 months to 24 months. The fraction activity for each subject was calculated using enzyme ontogeny. The predicted exposures were then binned in 1 kg bodyweight bins to evaluate the impact of dose levels on exposures across body weight bins.

### Example 13: Suspension Formulation Studies

Formulation composition and process variables of a liquid belumosudil formulation and having a concentration of 40 mg/mL, were studied to provide a homogeneous and stable dispersion. Process variables were evaluated to result in a robust suspension formulation.

Table 49 shows the initial formulation composition that was used to manufacture the first five lab batches (Batch Nos. 001 to 005) in the study.

TABLE 49
Initial formulation composition for Belumosudil
Suspension 40 mg/mL for Batch Nos. 001-005.

| | | | Concentration |
|---|---|---|---|
| No. | Material | Role | (% w/w) |
| 1 | Belumosudil mesylate | Active Pharmaceutical | 4.755 |
| | (Jet milled) | Ingredient | |
| 2 | Sodium benzoate | Anti-microbial agent | 0.049 |
| 3 | Sucralose | Sweetening agent | 0.147 |
| 4 | Syloid 244 FP | Suspending agent | 0.49 |
| 5 | PVP K-90 | Thickening agent | 4.902 |
| 6 | Tartaric acid | pH adjusting agent | q.s. to pH 3.0 |
| 7 | Purified water | Vehicle | q.s. to 100 |

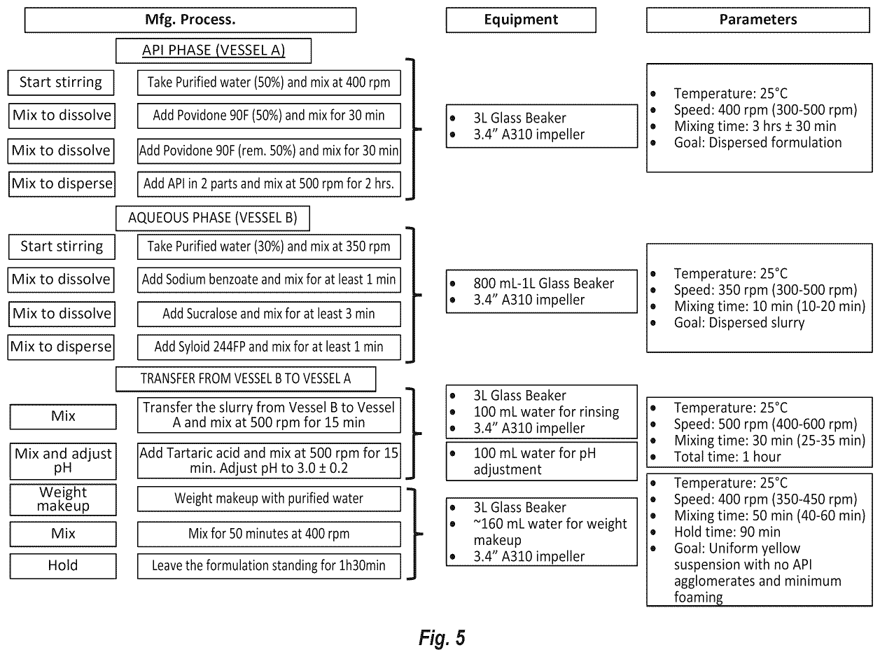

After manufacturing the first lab batch (Batch No. 001), undesired foaming and agglomeration were observed. Four additional lab batches were manufactured using the initial formulation composition with altered order of addition, including slow addition of the API and side phase preparation. The second lab batch (Batch No. 002) was prepared using the initial formulation composition shown in Table 49 and the manufacturing process shown in FIG. 5. However, foaming and agglomeration were also observed in the four additional batches (Batch Nos. 002 to 005). It was concluded that there was a need to reduce the foaming and agglomeration seen during the manufacturing process of the suspension to provide a liquid formulation suitable for commercial use.

An additional batch (Batch No. 006) of a belumosudil suspension having a concentration of 40 mg/mL was prepared using a modified manufacturing process. The modified process included replacing the A310 paddle impeller used for the mixing steps in the process with a Silverson homogenizer L5M-A fitted with a square hole high shear screen, and adding an anti-foaming agent to the formulation composition. A 2 kg lab batch of the belumosudil suspension with 2% w/w concentration of 30% Simethicone emulsion was manufactured using this Silverson homogenizer as Batch No. 006.

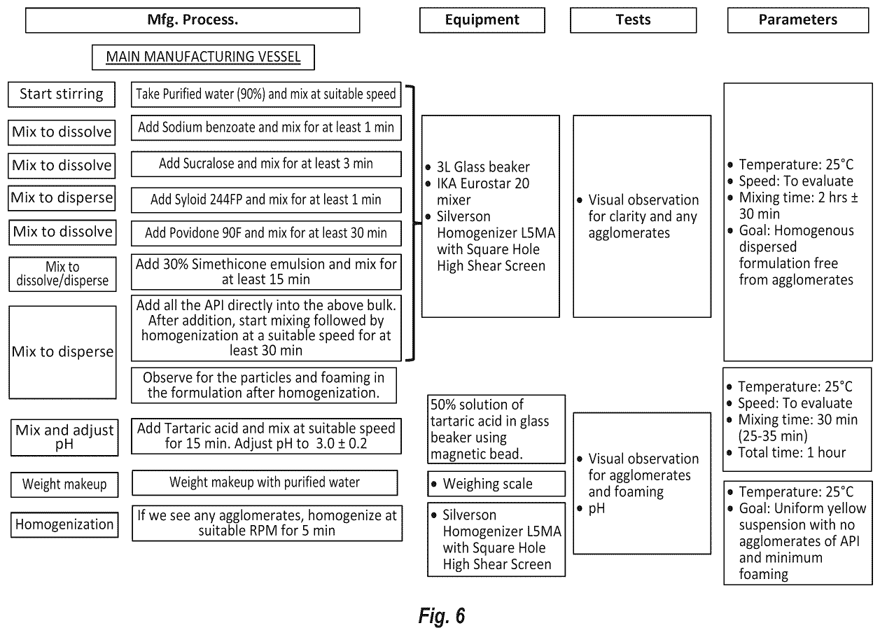

It was found that the modified process completely reduced the amount of foaming generated during manufacturing and provided a suspension suitable for commercial use. The modified formulation composition used in Batch No. 006 is shown in Table 50 and the modified manufacturing process used in Batch No. 006 is shown in FIG. 6.

TABLE 50
Modified formulation composition for
Belumosudil Suspension 40 mg/mL.

| | | | Concentration |
|---|---|---|---|
| No. | Material | Role | (% w/w) |
| 1 | Belumosudil mesylate | Active Pharmaceutical | 4.755 |
| | (Jet milled) | Ingredient | |
| 2 | Sodium benzoate | Anti-microbial agent | 0.049 |
| 3 | Sucralose | Sweetening agent | 0.147 |
| 4 | Syloid 244 FP | Suspending agent | 0.49 |
| 5 | PVP K-90 | Thickening agent | 4.902 |
| 6 | 30% Simethicone | Antifoaming agent | 2.00 |
| | emulsion (Medical | | |
| | Antifoam C Emulsion) | | |
| 7 | Tartaric acid | pH adjusting agent | q.s. to pH 3.0 |
| 8 | Purified water | Vehicle | q.s. to 100 |

The manufacturing process that was used to prepare belumosudil suspension 40 mg/mL for Batch No. 002 is shown in FIG. 5 and the initial formulation composition used for Batch No. 002 is shown in Table 49. The modified manufacturing process used to prepare belumosudil suspension 40 mg/mL for Batch No. 006 is shown in FIG. 6 and the modified formulation composition used in Batch No. 006 is shown in Table 50.

Table 51 compares manufacturing process details for Batch Nos. 002 and 006, showing multiple specific differences made between the initial and the modified manufacturing processes.

TABLE 51
Details of manufacturing processes followed for Belumosudil
Suspension 40 mg/mL for Batches 002 and 006.

| | Initial Manufacturing Process with | Modified Manufacturing Process with |
|---|---|---|
| | side phase | 30% simethicone emulsion |
| Step | (Batch No. 002) | (Batch No. 006) |
| 1 | Phase I: To 50% purified water, add | To 90% purified water, add sodium |
| | PVP K-90 in 2 parts under mixing at 500 | benzoate under mixing for 1 min. at |
| | RPM. Mix for 30 mins. after each | 500 RPM. |
| | addition. | |
| 2 | To this, add API in 2 parts under | To this, add sucralose under mixing for |
| | mixing at 500 RPM. After complete | 3 mins. at 500 RPM. |
| | addition, mix for 2 hours. | |
| 3 | Phase 2: To another 30% purified water, | To this, add Povidone (PVP K-90) |
| | add sodium benzoate, sucralose, and | under mixing 600 RPM. Mix for 30 |
| | Syloid 244 FP one after another. Mix | mins. |
| | after each addition. | |
| 4 | Add Phase 2 to Phase I under mixing. | To the above clear solution, add Syloid |
| | Mix for 15 mins. | 244 FP under mixing at 500 RPM for 1 |
| | | min. |
| 5 | Adjust the pH to 3.0 with tartaric acid | To this, add Simethicone emulsion |
| | and mix at appropriate speed for 15 | under homogenization and homogenize |
| | mins. | for 10 mins at 5000 RPM. (Silverson |
| | | Square Hole Screen) |
| 6 | Make up the weight to 2 kg with | Add all the API at once and start mixing |
| | purified water. Mix at 400 RPM for 50 | slowly to 600 RPM. Mix for 5 mins. |
| | mins. | Note: Ensure all the API from the vessel |
| | | walls and rotating shaft is mixed in the |
| | | suspension. |
| 7 | Allow the bulk to stand for 1 hour 30 | Homogenize the bulk for 30 mins at |
| | mins to dissipate the foam. | 5000 RPM using Square Hole Screen. |
| 8 | none | Adjust pH to 3.0 with tartaric acid and |
| | | mix at 550 RPM for 15 mins. |
| 9 | none | Weight make-up with purified water to |
| | | 2 kg and mix at 500 RPM for 10 mins. |

The manufacturing process shown in FIG. 5 that was used for preparing Batch No. 002 includes two phases (the “API phase” and the “aqueous phase”) using two vessels; one for mixing the povidone PVP and belumosudil mesylate ingredients, and the other for mixing the sodium benzoate, sucralose and Syloid colloidal silica ingredients. These two phases are mixed separately, then combined and the tartaric acid is added, the pH and water levels are adjusted, and the formulation is left standing for 1 hour and 30 minutes. Mixing is performed with a A310 paddle impeller rotating at speeds ranging from 300 to 600 rpm. According to Table 51, the hold time is used to allow foaming to dissipate, and the mixing time and hold time together add up to at least 305 minutes.

The manufacturing process shown in FIG. 6 that was used for preparing Batch No. 006 uses a single manufacturing vessel that is used for mixing all of the ingredients, and there is no hold time needed to dissipate foaming, as there is no foaming produced. Mixing is performed with a Silverson homogenizer fitted with a square hole high shear screen impeller rotating at speeds up to 5000 rpm. According to Table 51, the mixing time adds up to 105 minutes.

The use of the Silverson homogenizer allowed for much higher rotation speeds to be used in the mixing steps of the process, and use of the square hole high shear screen allowed for high shear rates to be reached during the mixing. The process using the Silverson homogenizer has shorter total mixing times and does not include a 90 minute hold step, as was needed in the process using the A310 paddle impeller and lower speed mixing. The suspension manufactured using the modified formulation composition including the simethicone emulsion and the Silverson homogenizer (Batch No. 006) had no foaming, unlike the suspension manufactured without Simethicone and using the A310 paddle impeller and low speed mixing (Batch No. 002) which exhibited foaming.

The liquid belumosudil oral suspension manufactured using the modified formulation composition including the simethicone emulsion and the Silverson homogenizer, described herein and as shown in FIG. 6, provided a robust homogeneous and stable dispersion that can be re-suspended homogenously, such that it can be shaken multiple times before administration, over time, with reproducible delivery of the desired drug dosage. During formulation manufacture, mixing at high speeds and with a specific paddle shape is generally used to reduce particle agglomeration and provide a homogeneous and stable dispersion. But a side effect of mixing at high speeds, especially with a hydrophobic drug product, is increased foaming. The modified composition and manufacturing process overcomes the foaming issue and results in a suspension with the desired features.

### Example 14: Additional Suspension Formulation Studies

Further studies can evaluate additional formulation and process variables of a belumosudil suspension having a concentration of 40 mg/mL. The relationship between input variables (factors) and output variables (responses) will be studied. Studies to be performed include evaluating effective concentrations for simethicone in the belumosudil suspension 40 mg/mL.

The formulation and process parameters to be evaluated will be based on Design of Experiments (DoE) conducted using Minitab 21 software. The formulation compositions to be studies are shown in Table 52. The study ranges for the formulation variables along with previously determined independent and identically distributed (IID) limits are also shown along with the compositions. Effective simethicone concentrations, the effects of pH, and effective concentrations of Syloid 244 FP and PVP K-90 will also be studied.

TABLE 52
Ranges for Formulation Variables Along with the IID Limits

| | mg/5 mL | Concentration | Study Range (%) | IID Limits | |
|---|---|---|---|---|---|
| Excipient | dose | (% w/w) | (Percent change) | (Oral Suspension) | |
| Belumosudil | 242.48 | 4.755 | 4.755 | not applicable | |
| mesylate | *(200.00 | | | | |
| | free base) | | | | |
| Sodium benzoate | 2.50 | 0.049 | 0.049 | 320 | mg |
| Sucralose | 7.50 | 0.147 | 0.147 | 264 | mg |
| Syloid 244 FP | 25.00 | 0.49 | 0.44-0.54 | 400 | mg |
| | | | (10% change) | | |
| PVP K-90 | 250.00 | 4.902 | 4.411-5.392 | 2 | mg |
| | | | (10% change) | | |
| 30% Simethicone | 102.00 | 2.00 | 0-2.00 | 240 | mg |
| Emulsion | | | | | |
| Tartaric acid | q.s. to pH | q.s. to pH 3.0 | q.s. to pH | 40 | mg |
| | 3.0 (3.825) | (approx. 0.075) | 2.7-3.3 | | |
| | | | (10% change) | | |

| | | | | |
|---|---|---|---|---|
| Purified water | q.s. to 100 | q.s. to 100 | q.s. to 100 | not applicable |

*Salt conversion factor 1.2124 i.e., 242.48 mg of Belumosudil mesylate drug substance is equivalent to 200 mg of free base.
Density of the suspension is 1.02 g/mL

To evaluate effective concentrations for the 30% Simethicone Emulsion, the Design Plan is a One Factor at a Time (OFAT) Approach.

The same manufacturing process as used for Batch No. 006 in Example 13 will be followed except with the change of addition sequence of PVP K-90 and Syloid 244 FP. The different experiments that will be performed for evaluating effective concentrations of 30% Simethicone emulsion are shown in Table 53.

TABLE 53
OFAT Trials for Evaluating Effective Concentrations of 30% Simethicone Emulsion.

| | Excipient | | | | | | | | |
|---|---|---|---|---|---|---|---|---|---|
| | Concentration (% w/w) | | | | | | | | |
| ID No. | F1 | F2 | F3 | F4 | F5 | F6 | F7 | F8 | F9 |
| Belumosudil | 4.755 | 4.755 | 4.755 | 4.755 | 4.755 | 4.755 | 4.755 | 4.755 | 4.755 |
| mesylate | | | | | | | | | |
| Sodium | 0.049 | 0.049 | 0.049 | 0.049 | 0.049 | 0.049 | 0.049 | 0.049 | 0.049 |
| benzoate | | | | | | | | | |
| Sucralose | 0.147 | 0.147 | 0.147 | 0.147 | 0.147 | 0.147 | 0.147 | 0.147 | 0.147 |
| Syloid | 0.49 | 0.49 | 0.49 | 0.49 | 0.49 | 0.49 | 0.49 | 0.49 | 0.49 |
| 244 FP | | | | | | | | | |
| PVP K-90 | 4.902 | 4.902 | 4.902 | 4.902 | 4.902 | 4.902 | 4.902 | 4.902 | 4.902 |
| 30% | 2.00 | 1.50 | 1.25 | 1.00 | 0.75 | 0.50 | 0.25 | 0.0 | final concn |
| Simethicone | | | | | | | | | determined |
| Emulsion | | | | | | | | | from F1-F8 |
| Tartaric | q.s. to | q.s. to | q.s. to | q.s. to | q.s. to | q.s. to | q.s. to | q.s. to | q.s. to |
| acid | pH 3.0 | pH 3.0 | pH 3.0 | pH 3.0 | pH 3.0 | pH 3.0 | pH 3.0 | pH 3.0 | pH 3.0 |
| Purified | q.s. | q.s. | q.s. | q.s. | q.s. | q.s. | q.s. | q.s. | q.s. |
| water | to 100 | to 100 | to 100 | to 100 | to 100 | to 100 | to 100 | to 100 | to 100 |
| Silverson | square | square | square | square | square | square | square | square | round |
| Screen Type | hole | hole | hole | hole | hole | hole | hole | hole | hole |
| | high | high | high | high | high | high | high | high | dis- |
| | shear | shear | shear | shear | shear | shear | shear | shear | integrating |

TABLE 54
Responses to be Studied

| No. | Analytical Test performed | Analytical Method No. |
|---|---|---|
| 1 | Appearance | Visual |
| 2 | pH | USP <791> |
| 3 | Density | USP <841> |
| 4 | Specific Gravity | USP <841> |
| 5 | Viscosity | 1942-TM-2226 (00P) |
| 6 | Assay of Belumosudil mesylate | 1942-TM-2236 (00P) |
| | (Top/Middle/Bottom) | |
| 7 | Free fraction solubility | to be determined |
| 8 | Related substances | 1942-TM-2236 (00P) |
| 9 | Preservative Content (Assay of | 1942-TM-2236 (00P) |
| | Sodium Benzoate) | |
| 10 | Dissolution | to be determined |
| 11 | Microscopy/Particle Size | to be determined |
| | Distribution | |
| 12 | Sedimentation volume | to be determined |

To study the effect of pH: The API is soluble at pH 2.0-3.0, therefore the effect of pH on the suspension can be studied. The most effective formulation composition from the results of the studies described above will be used in this study. Table 55 shows two additional experiments that will be conducted for studying pH ranges.

TABLE 55
OFAT Trials for studying pH range

| | Concentration (% w/w) | | |
|---|---|---|---|
| | Final | | |
| Excipient | Formulation | | |
| ID No. | (F1-F9) | F10 | F11 |
| Belumosudil | 4.755 | 4.755 | 4.755 |
| mesylate | | | |
| Sodium | 0.049 | 0.049 | 0.049 |
| benzoate | | | |
| Sucralose | 0.147 | 0.147 | 0.147 |
| Syloid 244 FP | 0.49 | 0.49 | 0.49 |
| PVP K-90 | 4.902 | 4.902 | 4.902 |
| 30% | final conc. | final conc. | final conc. |
| Simethicone | determined from | determined from | determined from |
| Emulsion | above study | above study | above study |
| Tartaric acid | q.s. to pH 3.0 | q.s. to pH 2.7 | q.s. to pH 3.3 |
| Purified water | q.s. to 100 | q.s. to 100 | q.s. to 100 |

The same responses as shown in Table 54 will also be studied for the pH effect study.

DoE Studies for Syloid 244 FP, PVP K-90 and pH: The DoE plan as shown in Table 56 will be used for studying the effect of amounts of Syloid 244 FP and PVP K-90 along with pH. Design Plan: Fractional Factorial Design with 3 factors, with the following Design Summary:
Factors: 3 Base Design: 3,4 Resolution: III
Runs: 8 Replicates: 1 Fraction: ½
Blocks: 1 Center pts (Total): 4
Design Table (randomized)

| Run | Block | A | B | C |
|---|---|---|---|---|
| 1 | 1 | + | + | + |
| 2 | 1 | 0 | 0 | − |
| 3 | 1 | − | − | − |
| 4 | 1 | + | − | − |
| 5 | 1 | − | − | − |
| 6 | 1 | 0 | 0 | + |
| 7 | 1 | 0 | 0 | + |
| 8 | 1 | 0 | 0 | − |

TABLE 56
DoE for studying Effect of Amounts of
Svloid 244 FP, PVP K-90 along with pH

| Run | Identifi- | Amount of | Amount of | Amount of |
|---|---|---|---|---|
| Order | cation No. | Syloid 244 FP | PVP K-90 | Tartaric acid |
| 1 | F12 | 0.54 | 5.39 | q.s. to pH 3.3 |
| 2 | F13 | 0.49 | 4.90 | q.s. to pH 2.7 |
| 3 | F14 | 0.44 | 4.41 | q.s. to pH 3.3 |
| 4 | F15 | 0.54 | 4.41 | q.s. to pH 2.7 |
| 5 | F16 | 0.44 | 5.39 | q.s. to pH 2.7 |
| 6 | F17 | 0.49 | 4.90 | q.s. to pH 3.3 |
| 7 | F18 | 0.49 | 4.90 | q.s. to pH 3.3 |
| 8 | F19 | 0.49 | 4.90 | q.s. to pH 2.7 |

The same responses as shown in Table 54 will also be studied for the DoE Studies for Syloid 244 FP, PVP K-90 and pH study.

The Process Design of Experiments: Process DoE will be performed on the modified manufacturing process shown in FIG. 6. The process parameters identified for study are mixing time and speed after PVP K-90 addition, and homogenization time and speed after API addition. Table 57 shows the DoE trials to be conducted for studying these process parameters during manufacturing of Belumosudil Suspension 40 mg/mL.

Design Plan: Fractional Factorial Design with 4 factors, with the following Design Summary:
Factors: 4 Base Design: 4,8 Resolution: IV
Runs: 11 Replicates: 1 Fraction: ½
Blocks: 1 Center pts (Total): 3
Design Table (randomized)

| Run | Block | A | B | C | D |
|---|---|---|---|---|---|
| 1 | 1 | + | − | + | − |
| 2 | 1 | 0 | 0 | 0 | 0 |
| 3 | 1 | 0 | 0 | 0 | 0 |
| 4 | 1 | − | − | − | − |
| 5 | 1 | 0 | 0 | 0 | 0 |
| 6 | 1 | − | − | − | − |
| 7 | 1 | + | + | + | + |
| 8 | 1 | − | − | − | − |
| 9 | 1 | − | − | − | − |
| 10 | 1 | − | − | − | + |
| 11 | 1 | − | + | − | − |

TABLE 57
DoE for studying Process Parameters

| | | Mixing | Mixing | | |
|---|---|---|---|---|---|
| | Identifi- | speed after | time after | | |
| Run | cation | PVP K-90 | PVP K-90 | Homogeniza- | Homogeniza- |
| Order | No. | addition | addition | tion Speed | tion Time |
| 1 | F20 | 800 | 20 | 6000 | 15 |
| 2 | F21 | 600 | 30 | 5000 | 30 |
| 3 | F22 | 600 | 30 | 5000 | 30 |
| 4 | F23 | 400 | 20 | 6000 | 45 |
| 5 | F24 | 600 | 30 | 5000 | 30 |
| 6 | F25 | 400 | 40 | 6000 | 15 |
| 7 | F26 | 800 | 40 | 6000 | 45 |
| 8 | F27 | 400 | 20 | 4000 | 15 |
| 9 | F28 | 400 | 40 | 4000 | 45 |
| 10 | F29 | 800 | 20 | 4000 | 45 |
| 11 | F30 | 800 | 40 | 4000 | 15 |

The same responses as shown in Table 54 will also be studied for the DoE Studies for Process Parameter study.

1. A liquid formulation comprising 2-{3-[4-(1H-indazol-5-ylamino)-2-quinazolinyl]phenoxy}-N-(propan-2-yl) acetamide or a pharmaceutically acceptable salt thereof in a diluent in combination with a suspending agent and a thickener.

2. The liquid formulation of claim 1, further comprising an antifoaming agent.

3. The liquid formulation of claim 2, wherein the antifoaming agent comprises an about 30% simethicone emulsion.

4. The liquid formulation of claim 1, further comprising one or more of a preservative, a pH adjuster, and a sweetening agent, wherein the liquid formulation has a pH in the range of about 2.5 to 4.0.

5. The liquid formulation of claim 1, wherein the 2-{3-[4-(1H-indazol-5-ylamino)-2-quinazolinyl]phenoxy}-N-(propan-2-yl) acetamide or pharmaceutically acceptable salt thereof is jet-milled or pin-milled.

6. The liquid formulation of claim 1, wherein the 2-{3-[4-(1H-indazol-5-ylamino)-2-quinazolinyl]phenoxy}-N-(propan-2-yl) acetamide or pharmaceutically acceptable salt thereof comprises 2-{3-[4-(1H-indazol-5-ylamino)-2-quinazolinyl]phenoxy}-N-(propan-2-yl) acetamide mesylate.

7. The liquid formulation of claim 4, wherein the preservative is sodium benzoate, the suspending agent is colloidal silica, the thickener is povidone, the pH adjuster is tartaric acid, and the sweetening agent is sucralose.

8. The liquid formulation of claim 1, further comprising a preservative and a pH adjuster, wherein the liquid formulation has a pH in the range of about 2.5 to 4.0.

9. (canceled)

10. The liquid formulation of claim 1, further comprising a flavoring agent.

11. (canceled)

12. The liquid formulation of claim 1, wherein the liquid formulation is homogenized.

13. The liquid formulation of claim 4, comprising about 2 to 8 weight percentage 2-{3-[4-(1H-indazol-5-ylamino)-2-quinazolinyl]phenoxy}-N-(propan-2-yl) acetamide or a pharmaceutically acceptable salt thereof, about 0.02 to 0.08 weight percentage preservative, about 0.1 to 0.4 weight percentage sweetening agent, about 0.2 to 0.8 weight percentage suspending agent, and about 1 to 8 weight percentage thickener.

14. (canceled)

15. (canceled)

16. (canceled)

17. (canceled)

18. (canceled)

19. The liquid formulation of claim 1, formulated to deliver a dose of 2-{3-[4-(1H-indazol-5-ylamino)-2-quinazolinyl]phenoxy}-N-(propan-2-yl) acetamide or a pharmaceutically acceptable salt thereof to a patient in the range of a dose equivalent to about 50 to 400 mg of the free base in a volume of about 1 to 10 mL.

20. (canceled)

21. (canceled)

22. A method for treating graft-versus-host disease in a subject comprising administering to the subject a therapeutically effective amount of a liquid formulation of claim 1.

23. The method of claim 22, wherein the graft-versus-host disease is chronic graft-versus-host disease.

24. The method according to claim 22, wherein the subject is an adult patient.

25. The method according to claim 22, wherein the subject is a pediatric patient.

26. The method of claim 22, wherein the liquid formulation is administered to the subject at a dose of 2-{3-[4-(1H-indazol-5-ylamino)-2-quinazolinyl]phenoxy}-N-(propan-2-yl) acetamide or pharmaceutically acceptable salt thereof that is adjusted based on the body weight of the patient.

27. (canceled)

28. The method of claim 26, wherein the liquid formulation is administrated at a dose of 2-{3-[4-(1H-indazol-5-ylamino)-2-quinazolinyl]phenoxy}-N-(propan-2-yl) acetamide or pharmaceutically acceptable salt thereof, equivalent to between about 2.5 and 5.0 mg/kg of the free base.

29. (canceled)

30. (canceled)

31. (canceled)

32. A process for preparing a liquid formulation comprising 2-{3-[4-(1H-indazol-5-ylamino)-2-quinazolinyl]phenoxy}-N-(propan-2-yl) acetamide or a pharmaceutically acceptable salt thereof comprising mixing jet-milled and/or pin-milled 2-{3-[4-(1H-indazol-5-ylamino)-2-quinazolinyl]phenoxy}-N-(propan-2-yl) acetamide or a pharmaceutically acceptable salt thereof, in a diluent with a suspending agent and a thickener.

33. (canceled)

34. (canceled)

35. (canceled)

36. (canceled)

37. (canceled)

38. (canceled)

39. (canceled)

40. (canceled)

41. (canceled)

42. A liquid formulation comprising 2-{3-[4-(1H-indazol-5-ylamino)-2-quinazolinyl]phenoxy}-N-(propan-2-yl) acetamide or a pharmaceutically acceptable salt thereof produced by the process of claim 32.

43. A kit for preparing a liquid formulation of 2-{3-[4-(1H-indazol-5-ylamino)-2-quinazolinyl]phenoxy}-N-(propan-2-yl) acetamide or a pharmaceutically acceptable salt thereof comprising (a) in a first container, an amount of 2-{3-[4-(1H-indazol-5-ylamino)-2-quinazolinyl]phenoxy}-N-(propan-2-yl) acetamide or a pharmaceutically acceptable salt thereof; and (b) in a second container, a reconstitution vehicle comprising a suspending agent and a thickener in a diluent.

44. (canceled)

45. (canceled)

46. The liquid formulation of claim 1, wherein the 2-{3-[4-(1H-indazol-5-ylamino)-2-quinazolinyl]phenoxy}-N-(propan-2-yl) acetamide or a pharmaceutically acceptable salt thereof is at a concentration which is equivalent to about 40 mg/ml of the free base.
